(19) United States
(12) Patent Application Publication
(10) Pub. No.: US 2012/0083419 A1
(43) Pub. Date: Apr. 5, 2012

(54) Method and compositions for inhibiting tumorigenesis

(76) Inventors: Ariel Ruiz i Altaba, Geneva (CH); Sumana Datta, Bryan, TX (US); Milton Datta, Atlanta, GA (US)

(21) Appl. No.: 12/291,229

(22) Filed: Nov. 7, 2008

Related U.S. Application Data
(63) Continuation of application No. 10/927,951, filed on Aug. 29, 2004, now abandoned.
Application No. 10/927,951 is a continuation-in-part of application No. 10/456,954, filed on Jun. 6, 2003, now abandoned.
Application No. 10/456,954 is a continuation-in-part of application No. 09/825,155, filed on Apr. 3, 2001, now abandoned.
Application No. 09/825,155 is a continuation of application No. 09/102,491, filed on Jun. 22, 1998, now Pat. No. 6,238,876.
Application No. 10/927,951 is a continuation-in-part of application No. 10/414,267, filed on Apr. 15, 2003, now abandoned.
(60) Provisional application No. 60/050,286, filed on Jun. 20, 1997.
(60) Provisional application No. 60/372,508, filed on Apr. 15, 2002.

(51) Int. Cl. C40B 30/04; C12Q 1/68; G01N 33/566; C12N 5/09; C12N 5/071

(52) U.S. Cl. 506/9; 435/375; 435/7.1; 435/6.11; 435/6.12

(57) ABSTRACT

The present invention relates to compounds, small interfering RNAs and compositions and methods of inhibiting tumorigenesis and methods of inhibiting tumor cell growth and proliferation using agents that inhibit the hedgehog and Gli signaling pathway, including agents that inhibit GLI synthesis and/or function. The present invention also relates to particular biomarkers that can be used in the diagnosis and prognosis of prostate cancer. Methods of treating cancer, including prostate cancer are also provided using small organic compounds, siRNAs and blocking antibodies that inhibit or block the SHH/GLI pathway.

### CROSS-REFERENCE TO RELATED APPLICATIONS

The present application is a continuation of U.S. Ser. No. 10/927,951, filed Aug. 29, 2004 which is a Continuation-in-Part of U.S. Ser. No. 10/456,954, filed Jun. 6, 2003, which is a Continuation-in-Part of non-provisional application U.S. Ser. No. 09/825,155, filed Apr. 3, 2001, which is a Continuation of U.S. Ser. No. 09/102,491, filed Jun. 22, 1998, now U.S. Pat. No. 6,238,876, which claims benefit of priority to provisional application 60/050,286, filed Jun. 20, 1997; and is also a Continuation-in-Part of non-provisional application U.S. Ser. No. 10/414,267, filed Apr. 15, 2003, which claims the benefit of priority to provisional application U.S. Ser. No. 60/372,508, filed Apr. 15, 2002. Applicants claim the benefit of all of the above applications under 35 U.S.C. §119(e) and 35 U.S.C. §120, and the disclosures of all of the above applications are incorporated herein by reference in their entireties.

### GOVERNMENTAL SUPPORT

The research leading to the present invention was supported, at least in part, by grants from the National Institute of Neurological Disorders and Stroke, Grant No. R01 NS-37352, and from the National Cancer Institute, Grant No. R01 CA78736. Accordingly, the Government may have certain rights in the invention.

### FIELD OF THE INVENTION

The present invention relates generally to the diagnosis and treatment of pathologies involving tumor formation, cancer and neoplasia, and more particularly to compositions and methods of inhibiting tumorigenesis, tumor growth and tumor survival using agents that inhibit the sonic hedgehog (SHH or HH) and GLI signaling pathway. The present invention also relates to particular biomarkers that can be used in the diagnosis and prognosis of cancer. Methods of treating cancer, including but not limited to prostate cancer are also provided through the use of relevant therapeutic agents based on their effect on the level of expression and/or activity of GLI genes. Small organic compounds, siRNAs and blocking antibodies that inhibit or block the SHH/GLI pathway are contemplated for the preparation of therapeutic compositions and methods of use.

### BACKGROUND OF THE INVENTION

Inductive signaling plays a critical role in both normal and disease development as developmental pathways that become unregulated in the adult can lead to abnormal patterning, overproliferation and neoplasia. One signaling pathway that is involved in several patterning events during embryogenesis is that triggered by secreted sonic hedgehog (Shh) (Echelard, Y., Epstein, D. J., St-Jacques, B., Shen, L., Mohler, J., McMahon, J. A. & McMahon, A. P., Cell 75, 1417-1430 (1993); Riddle, R., Johnson, R. L., Laufer, E. & Tabin, C., Cell 75, 1401-1418 (1993); Krauss, S., Concordet, J.-P. & Ingham, P. W, Cell 75, 1431-1444 (1993); Roelink, H., Augsburger, A., Heemskerk, J., Korzh, V., Norlin, S., Ruiz i Altaba, A., Tanabe, Y., Placzek, M., Edlund, T., Jessell, T. M. & Dodd, J., Cell 76, 761-775 (1994)). Shh binding to the membrane patched (ptc)-smoothened (smo) receptor complex elicits a cascade of cytoplasmic signal transduction events, including the inhibition of protein kinase A (PKA) (Fan, C.-M., Porter, J. A., Chiang, C., Chang, D. T., Beachy, P. A. & Tessier-Lavigne, M., Cell 81, 457-465 (1995); Hynes, M., Porter, J. A., Chiang, C., Chang, D., Tessier-Lavigne, M., Beachy, P. A. & Rosenthal, A., Neuron 15, 35-34 (1995); Concordet, J.-P., Lewis, K. E., Moore, J., Goodrich, L. V., Johnson, R. L., Scott, M. P. & Ingham, P. W., Development 122, 2835-2846 (1996); Epstein, D. J., Marti, E., Scott, M. P. & McMahon, A. P., Development 122, 2885-2894 (1996); Goodrich, L. V., Johnson, R. L., Milenkovic, L., McMahon, J. A. & Scott, M. P., Genes Dev. 10, 301-312 (1996); Hammerschmidt, M., Bitgood, M. J. & McMahon, A. P., Genes and Dev. 10, 647-658 (1996); Marigo, V., Johnson, R. L., Vortkamp, A. & Tabin, C. J., Dev. Biol. 180, 273-283 (1996); Stone, D. M., Hynes, M., Armanini, M., Swanson, T. A., Gu, Q., Johnson, R. L., Scott, M. P., Pennica, D., Goddard, A., Phillips, H., Noll, M., Hooper, J. E., de Sauvage, F. & Rosenthal, A., Nature 384, 129-134 (1996)) that leads to the transcription of the zinc finger transcription factor gene Gli1 (Marigo, V., Johnson, R. L., Vortkamp, A. & Tabin, C. J., Dev. Biol. 180, 273-283 (1996); Lee, J., Platt, K. A., Censullo, P. & Ruiz i Altaba, A., Development (1997)). Gli1 is a proto-oncogene first isolated as an amplified gene in a glioma that can transform fibroblasts in cooperation with E1A (Kinzler, K. W., Bigner, S. H., Bigner, D. D., Trent, J. M., Law, M. L., O'Brien, S. J., Wong, A. J. & Vogelstin, B., Science 236, 70-73 (1987); Ruppert, J. M., Vogelstein, B. & Kinzler, K. W., Molecular and Cellular Biology 11, 1724-1728 (1991)). Gli1 is a member of a family comprising two other related genes: Gli2 and Gli3 (Ruppert, J. M., Vogelstein, B., Arheden, K. & Kinzler, K. W., Mol. Cell. Biol. 10, 5408-5415 (1990); Hui, C.-C., Slusarski, D., Platt, K. A., Holmgren, R. & Joyner, A. L., Developmental Biology 162, 402-413 (1994)). However, only Gli1 has been shown to be a target of Shh and mimic its effects (Lee, J., Platt, K. A., Censullo, P. & Ruiz i Altaba, A., Development (1997)). In Drosophila, hedgehog signaling (Forbes, A. J., Nakano, Y., Taylor, A. M. & Ingham, P. W., Development Supplement 115-124 (1993)) similarly leads to the action of cubitus interruptus (ci), a Gli homolog that activates transcription of hedgehog-target genes (Domínguez, M., Brunner, M., Hafen, E. & Basler, K., Science 272, 1621-1625 (1996); Alexandre, C., Jacinto, A. & Ingham, P. W., Genes and Dev. 10, 2003-2013 (1996); Hepker, J., Wang, Q.-T., Motzny, C. K., Holmgren, R. & Orenic, T. V., Development 124, 549-558 (1997); von Ohnen, T., Lessing, D., Nusse, R. & Hooper, J. E., Proc. Natl. Acad. Sci. USA. 94, 2404-2409 (1997); Mullor, J. L., Calleja, M., Capdevila, J. & Guerrero, I., Development 124, 1227-1237 (1997)).

One of the processes in which Shh signaling is involved is the differentiation of ventral neural tube cell types acting as a notochord and floor plate-derived signal (Echelard, Y., Epstein, D. J., St-Jacques, B., Shen, L., Mohler, J., McMahon, J. A. & McMahon, A. P., Cell 75, 1417-1430 (1993); Roelink, H., Augsburger, A., Heemskerk, J., Korzh, V., Norlin, S., Ruiz i Altaba, A., Tanabe, Y., Placzek, M., Edlund, T., Jessell, T. M. & Dodd, J., Cell 76, 761-775 (1994); Marti, E., Bumcrot, D. A., Takada, R. & McMahon, A. P., Nature 375, 322-325 (1995); Ruiz i Altaba, A., Roelink, H. & Jessell, T. M., Mol. Cell. Neurosci. 6, 106-121 (1995); Chiang, C., Litingtung, Y., Lee, E., Young, K. E., Corden, J. L., Westphal, H. & Beachy, P. A., Nature 383, 407-413 (1996); Ericson, J., Morton, S., Kawakami, A., Roelink, H. & Jessell, T. M., Cell 87, 661-673 (1996)). Previous work by the applicants herein on the role of sonic hedgehog signaling during neural plate patterning in frog (Xenopus laevis) embryos demonstrated that cells becoming floor plate respond to Shh by expressing Gli1, Pintallavis and HNF-3β, critical transcription factors that themselves can induce the differentiation of floor plate cells (Lee, J., Platt, K. A., Censullo, P. & Ruiz i Altaba, A. Gli1 is a target of sonic hedgehog that induces ventral neural tube development. Development, 124(13):2537-52 (1997); Ruiz i Altaba, A., Roelink, H. & Jessell, T. M. Restrictions to Floor Plate Induction by hedgehog and Winged Helix Genes in the Neural Tube of Frog Embryos. Mol. Cell. Neurosci. 6, 106-121 (1995); Ruiz i Altaba, A., Cox, C., Jessell, T. & Klar, A. Deregulated Expression of the Midline Transcription Factor Pintallavis Induces Ectopic Expression of a Floor Plate Marker. Proc. Natl. Acad. Sci. USA 90, 8268-8272 (1993); Ruiz i Altaba, A., Prezioso, V. R., Darnell, J. E. & Jessell, T. M. Sequential expression of HNF-3β and HNF-3 by embryonic organizing centers: the dorsal lip/node, notochord and floor plate. Mech. Dev. 44, 91-108 (1993)).

In addition to effects on neural tissue, it has been found that ectopic expression of Shh and Gli1 also leads to the activation of Shh signaling target genes in epidermal non-neural ectoderm. Injected Shh induced the ectopic expression of Gli1, HNF-3β and Shh (Ruiz i Altaba, A., Roelink, H. & Jessell, T. M., Mol. Cell. Neurosci. 6, 106-121 (1995)), and ectopic expression of Gli1 induced the ectopic expression of HNF-313 and Shh (Lee, J., Platt, K. A., Censullo, P. & Ruiz i Altaba, A. Gli1 is a target of sonic hedgehog that induces ventral neural tube development. Development (1997)). Together, these results indicated that both neural and epidermal cells have functional reception and transduction mechanisms for Shh and can respond by activating the expression of Shh/Gli1 target genes even though epidermal cells do not normally receive the Shh signal at this stage.

Furthermore, SHH signaling has been implicated in many aspects of animal development, acting through the transmembrane proteins PATCHED1 (PTCH1) and SMOH to activate the GLI zinc-finger transcription factors (Ingham, P. & McMahon, A., Genes Dev. 15, 3059-87 (2001); Ruiz i Altaba, A., Sanchez, P. & Dahmane, N., Nat. Rev. Cancer 2, 361-372 (2002).

Prostate cancer (PC) may be the most common solid tumor in men (Nelson, W. G., De Marzo, A. M. & Isaacs, W. B., N. Engl. J. Med. 349, 366-381 (2003)). It is generally acknowledged in the medical community that all men will eventually develop prostate cancer, provided that they live long enough for the condition to develop. For example, 50% of all men over 50, and essentially all men over 70 suffer from some form of prostate hyperplasia. Indeed, prostate cancer is the most frequently diagnosed cancer in the United States, with over a quarter of a million new cases being diagnosed each year. Despite the roughly $4 billion dollars per year spent treating this disease, forty thousand men die every year due to prostate cancer, which makes prostate cancer the second leading cause of cancer death in men.

Inhibition of testosterone dependent tumor growth has been the mainstay of clinical treatment for advanced disease, but tumor escape is typical, with subsequent testosterone independent growth leading to patient death (Martel, C. L., Martel, C. L., Gumerlock, P. H., Meyers, F. J. & Lara, P. N., Cancer Treat Rev. 29, 171-187 (2003)). An understanding of the molecular mechanisms underlying PC, and thus of additional therapeutic approaches, has remained elusive. This is due in part to the scarcity of appropriate human prostate cell models given the very difficult task of growing PC cells (Rhim, J. S., Prostate Cancer Prostatic Dis. 3, 229-235 (2000)).

One of the primary characteristics of prostate cancer is that it generally arises relatively late in life and then progresses slowly. If this were always true, the optimal medical response might be to simply monitor the progression of the cancer rather than aggressively treating it, since by the time the cancer progressed to a life threatening stage, the patient would have likely expired due to other more rapidly progressing factors. Unfortunately, this strategy is not always successful, since prostate cancers are highly heterogeneous in their progression. Some prostate cancers grow very rapidly, thus necessitating the need for more aggressive treatment, while others may grow very slowly and are not life-threatening. Accordingly, perhaps one of the more important considerations in current treatment strategies of prostate cancer may be distinguishing aggressive prostate cancers, which need more aggressive treatment regimens, from less aggressive ones, which only require monitoring.

Currently, there are no accurate prognostic tests or an effective means to distinguish aggressive prostate cancers from slow-growing prostate cancers. The present technology relies on monitoring levels of the protein PSA. Unfortunately, this method results in a high percentage of false positives. Furthermore, it cannot be used as a predictor of future disease progression.

Considering the adverse side-effects and expense associated with treating cancer, in particular, prostate cancer, better diagnostic and prognostic tools are needed. Therefore, there is a need to identify other factors that can be used for the early detection of cancer, as well as the means to predict the progression of cancer, including but not limited to, prostate cancer. In addition, there is a need to identify new therapeutic strategies for treating cancer, including, but not limited to, prostate cancer.

The citation of any reference herein should not be construed as an admission that such reference is available as “Prior Art” to the instant application.

### SUMMARY OF THE INVENTION

The human zinc finger transcription factors GLI1, GLI2 and GLI3 encode downstream effectors of the Sonic hedgehog pathway, which play critical roles during early development, organogenesis and in the adult, particularly in development of the nervous system. GLI1 has also been implicated in a number of cancers, including basal cell carcinoma, rhabdomyosarcoma, medulloblastoma, gliomas and small cell lung tumors. The studies presented herein provide further evidence for GLI1, GLI2 and GLI3 in tumorigenesis, particularly prostate cancer. Since cancer may be a disease of stem cell lineages and SHH-GLI signaling controls the behavior of precursors and of cells with stem cell properties in the mammalian brain (Lai K, Kaspar B K, Gage F H & Schaffer D V, Nat Neurosci 6, 21-7 (2003); Machold R, et al., Neuron 39,937-50 (2003); Palma, V & Ruiz i Altaba, A., Development 131, 337-345 (2004)), as well as in other organs and species (Zhang Y & Kalderon D., Nature. 410, 599-604 (2001); Park Y, et al., Dev Biol. 253, 247-57 (2003)), the inventors propose that many cancers, including those disclosed herein, in particular, prostate cancer, derives from inappropriate expansion of stem cell lineages due to abnormal SHH-GLI function. That is, while the SHH/GLI pathway plays a positive role in terms of promoting the generation of adult neuronal cells from stem cells, thus providing a means for generation of populations of neuronal cells useful for treatment of various neurodegenerative diseases, this signaling pathway also has a deleterious effect on cells in terms of promotion of tumorigenesis. Therefore, the present invention describes novel strategies to prevent the tumorigenic effects of the SHH/GLI pathway.

Accordingly, it is an object of the present invention to develop novel therapeutic strategies for treatment of cancers using agents that prevent signaling through the SHH/GLI pathway. Furthermore, as shown by Applicants herein, it would be advantageous to develop a means of determining whether a subject is suffering from a hyperproliferative condition or a cancerous condition, in particular, prostate cancer, by measurement of GLI in tumor samples or other body tissues, cells or body fluids as a means of assessing the presence of various cancers, which could then aid in developing the most effective treatment regimen. Thus, a GLI protein or a nucleic acid encoding a GLI protein may be used as a biomarker for the presence of a cancerous or hyperproliferative condition.

A first aspect of the invention provides a method for inhibiting the synthesis and/or expression and/or activity of a GLI protein in a cell in vitro or in vivo comprising, contacting said cell with, or introducing into said cell an inhibitor or antagonist of GLI synthesis and/or expression and/or activity.

In one particular embodiment, the inhibitor or antagonist is selected from the group consisting of a small molecule inhibitor or antagonist of GLI synthesis and/or expression and/or activity, an antisense molecule or a siRNA molecule that is essentially complementary to at least a part of the sequence of a nucleic acid encoding the GLI protein, antibodies to GLI, and agents that exhibit mimicry to GLI or antagonism to it or control over its production. In another particular embodiment, the contacting of the cell or the introducing the inhibitor or antagonist into the cell results in inhibition of synthesis and/or expression and/or activity of the GLI protein in said cell.

In another particular embodiment, the contacting of the cell or the introducing the inhibitor or antagonist into the cell results in inhibition of cellular proliferation, inhibition of cancer/tumor cell growth and/or survival and/or inhibition of tumorigenesis. Thus, another particular embodiment provides for inhibition and/or antagonism of the SHH/GLI pathway by the agents of the present invention in a tumor cell. In yet another particular embodiment, the tumor cells in which said agents may be effective are tumor cells selected from the group consisting of gliomas, medulloblastomas, primitive neuroectodermal tumors (PNETS), basal cell carcinoma, small cell lung cancers, large cell lung cancers, tumors of the gastrointestinal tract, rhabdomyosarcomas, soft tissue sarcomas, pancreatic tumors, bladder tumors and prostate tumors. In yet another particular embodiment, the GLI protein is selected from the group consisting of GLI1, GLI2 and GLI3.

In another particular embodiment, the antagonist comprises an antisense compound comprising an oligonucleotide of about 10 to 50 nucleobases in length targeted to a nucleic acid encoding a GLI protein, wherein said antisense compound inhibits the expression and/or activity of said GLI protein. In yet another particular embodiment, the antagonist comprises an antisense compound comprising an oligonucleotide of about 10 to 30 nucleobases in length targeted to a nucleic acid encoding a GLI protein, wherein said antisense compound inhibits the expression and/or activity of said GLI protein. In yet another particular embodiment, the antagonist comprises an antisense compound comprising an oligonucleotide of about 17 to 25 nucleobases in length targeted to a nucleic acid encoding a GLI protein, wherein said antisense compound inhibits the expression and/or activity of said GLI protein. In yet another particular embodiment, the antisense compound has the sequence as set forth in SEQ ID NO: 33.

In another particular embodiment, the antagonist comprises a siRNA molecule, said siRNA molecule comprising a double stranded structure having a nucleotide sequence which is substantially identical to or complementary to at least a part of the gli gene in an amount sufficient to inhibit the synthesis and/or expression and/or activity of a gli gene. In yet another particular embodiment, the gli gene may be selected from the group consisting of gli1, gli2 and gli3. In yet another particular embodiment, the siRNA hybridizes under standard conditions to the gli gene. In yet another particular embodiment, the siRNA molecule is about 10 to 50 nucleotides in length. In yet another particular embodiment, the siRNA molecule is about 10 to 30 nucleotides in length. In yet another particular embodiment, the siRNA molecule is about 17 to 25 nucleotides in length. In yet another particular embodiment, the siRNA molecule is selected from the group consisting of the nucleic acid sequences set forth in SEQ ID NO: 29, SEQ ID NO: 31 and SEQ ID NO: 32.

In another particular embodiment, the antagonist is a small molecule inhibitor of GLI expression and/or synthesis and/or activity. In yet another particular embodiment, the small molecule inhibitor is a plant alkaloid or its analogs or derivatives thereof. In yet another particular embodiment, the plant alkaloid is selected from cyclopamine, jervine and their analogs or derivatives thereof.

In another particular embodiment, the antagonist is an antibody specific for at least one of the GLI proteins. In yet another particular embodiment, the antibody may be a polyclonal antibody or a monoclonal antibody. The antibody may be a “chimeric antibody”, which refers to a molecule in which different portions are derived from different animal species, such as those having a human immunoglobulin constant region and a variable region derived from a murine mAb. (See, e.g., Cabilly et al., U.S. Pat. No. 4,816,567; and Boss et al., U.S. Pat. No. 4,816,397.). The antibody may be a human or a humanized antibody. The antibody may be a single chain antibody. (See, e.g., Curiel et al., U.S. Pat. No. 5,910,486 and U.S. Pat. No. 6,028,059). The antibody may be prepared in, but not limited to, mice, rats, guinea pigs, rabbits, goats, sheep, swine, dogs, cats, or horses. It may be may be an Fab fragment or a soluble component thereof. In yet another particular embodiment, the invention provides for an immortal cell line that produces the antagonistic monoclonal antibody. In yet another particular embodiment, the antibody is a blocking antibody and is effective as a therapeutic agent to prevent the proliferation of tumor cells in vivo. In yet another particular embodiment, the antibody may be coupled to an anti-tumor drug or radioisotope for use in treating tumor cells in situ. The antibody may be effective as a diagnostic agent to aid in identification of tumor cells in situ or in vivo. The antibody may be coupled to an imaging reagent and is used for imaging of tumor cells in situ or in vivo.

A second aspect of the invention provides a method of preventing and/or treating cellular debilitations, derangements and/or dysfunctions and/or hyperplastic/hyperproliferative and/or cancerous disease states, and metastasis of tumor cells in a mammal characterized by the presence and/or expression of a gli gene or gene product, such as a GLI protein, comprising administering to a mammal a therapeutically effective amount of an agent that inhibits or antagonizes the synthesis and/or expression and/or activity of a GLI molecule.

In one particular embodiment, the inhibitors/antagonists are selected from the group consisting of small molecule antagonists of GLI expression and activity, antisense compounds, siRNA nucleic acids, antibodies to GLI, and agents that exhibit mimicry to GLI or antagonism to it or control over its production. In another particular embodiment, the antagonist comprises agents that increase negative, or decrease positive acting elements affecting SHH-GLI signaling, said agents selected from the group consisting of small molecules that alter PKA kinase activity, small molecules that alter GSK3 kinase activity, small molecules that alter CK1 kinase activity and small molecules or siRNAs/antisense RNAs that alter DYRK1 kinase activity. In yet another particular embodiment the GLI molecule is selected from the group consisting of GLI1, GLI2 and GLI3.

In another particular embodiment, the hyperproliferative and/or cancerous disease states are selected from the group consisting of gliomas, medulloblastomas, primitive neuroectodermal tumors (PNETS), basal cell carcinoma, small cell lung cancers, large cell lung cancers, tumors of the gastrointestinal tract, rhabdomyosarcomas, soft tissue sarcomas, pancreatic tumors, bladder tumors and prostate tumors.

In another particular embodiment, the antagonist comprises an antisense compound comprising an oligonucleotide of about 10 to 50 nucleobases in length targeted to a nucleic acid encoding a GLI protein, wherein said antisense compound inhibits the expression and/or activity of said GLI protein. In yet another particular embodiment, the antagonist comprises an antisense compound comprising an oligonucleotide of about 10 to 30 nucleobases in length targeted to a nucleic acid encoding a GLI protein, wherein said antisense compound inhibits the expression and/or activity of said GLI protein. In another particular embodiment, the antagonist comprises an antisense compound comprising an oligonucleotide of about 17 to 25 nucleobases in length targeted to a nucleic acid encoding a GLI protein, wherein said antisense compound inhibits the expression and/or activity of said GLI protein. In another particular embodiment, the antisense compound has the sequence as set forth in SEQ ID NO: 33. In another particular embodiment, the antisense compound, upon administering to said mammal, results in the inhibition of expression and/or activity of said GLI protein in the cells of said mammal. In another particular embodiment, the delivery of said antisense compound to said mammal further results in inhibition of cellular proliferation in said mammal.

In another particular embodiment, the antagonist comprises a siRNA molecule, said siRNA molecule comprising a double stranded structure having a nucleotide sequence which is substantially identical to at least a part of the gli gene in an amount sufficient to inhibit the synthesis and/or expression and/or activity of a gli gene. In another particular embodiment, the gli gene may be selected from the group consisting of gli1, gli2 and gli3. In another particular embodiment, the siRNA hybridizes under stringent conditions to the gli gene. In another particular embodiment, the siRNA molecule is about 10-50 nucleotides in length. In another particular embodiment, the siRNA molecule is about 10-30 nucleotides in length. In yet another particular embodiment, the siRNA molecule is about 17-25 nucleotides in length. In yet another particular embodiment, the siRNA molecule may be selected from the group consisting of the nucleic acid sequences set forth in SEQ ID NO: 29, SEQ ID NO: 31 and SEQ ID NO: 32.

In another particular embodiment, the antagonist is a small molecule inhibitor of GLI expression and/or synthesis and/or activity. In another particular embodiment, the small molecule inhibitor is a plant alkaloid or its analogs or derivatives thereof. In another particular embodiment, the plant alkaloid is selected from cyclopamine, jervine and/or their analogs or derivatives thereof.

In another particular embodiment, the antagonist is an antibody specific for at least one of the GLI proteins. In yet another particular embodiment, the antibody may be a polyclonal antibody or a monoclonal antibody. The antibody may be a single chain antibody. It may be a chimeric antibody. It may be may be an Fab fragment or a soluble component thereof. It may be a human or humanized antibody. It may be produced in other animals, including but not limited to horses, goats, sheep, mice, rats, rabbits and guinea pigs. In yet another particular embodiment, the invention provides for an immortal cell line that produces the antagonistic monoclonal antibody. In yet another particular embodiment, the antibody is a blocking antibody and is effective as a therapeutic agent to prevent the proliferation of tumor cells in vivo. In yet another particular embodiment, the antibody may be coupled to an anti-tumor drug or radioisotope for use in treating tumor cells in situ. The antibody may be effective as a diagnostic agent to aid in identification of tumor cells in situ or in vivo. The antibody may be coupled to an imaging reagent and is used for imaging of tumor cells in situ or in vivo.

A third aspect of the invention provides a method of treating apoptotic resistant tumor cells comprising administering an antagonist of the SHH/GLI signaling pathway to said tumor cell in vitro or in vivo. In one embodiment, the method comprises the use of an antagonist or inhibitor of the SHH/GLI pathway as a means of inducing a tumor cell to undergo senescence, apoptosis, or necrosis. In another embodiment, said administering results in tumor cell death and prevention from metastasis. In another particular embodiment, the antagonist comprises agents that increase negative, or decrease positive acting elements affecting SHH-GLI signaling, said agents selected from the group consisting of small molecules that alter PICA kinase activity, small molecules that alter GSK3 kinase activity and small molecules that alter CK1 kinase activity, and small molecules or siRNAs/antisense RNAs that alter DYRK1 kinase activity. In yet another particular embodiment, the tumor cell is selected from the group consisting of brain tumors, e.g. gliomas, medulloblastomas, primitive neuroectodermal tumors (PNETS); skin tumors, e.g. basal cell carcinoma, lung tumors, e.g. lung adenocarcinomas, small cell lung cancers, large cell lung cancers; tumors of the gastrointestinal tract; muscle tumors (rhabdomyosarcomas); soft tissue tumors (e.g. sarcomas), pancreatic tumors, bladder tumors and prostate tumors.

In another particular embodiment, the inhibitor or antagonist is selected from the group consisting of a small molecule inhibitor or antagonist of GLI synthesis and/or expression and/or activity, an antisense molecule or a siRNA molecule that is essentially complementary to at least a part of the sequence of a nucleic acid encoding the GLI protein, antibodies to GLI, and agents that exhibit mimicry to GLI or antagonism to it or control over its production.

In another particular embodiment, the antisense molecule comprises the sequence as set forth in SEQ ID NO: 33. In another particular embodiment, the siRNA molecule may be selected from the group consisting of the nucleic acid sequences of SEQ ID NO: 29, SEQ ID NO: 31 and SEQ ID NO: 32. In another particular embodiment, the small molecule inhibitor of GLI synthesis and/or expression and/or activity is a plant alkaloid such as, but not limited to, cyclopamine, jervine and/or analogs or derivatives thereof.

A fourth aspect of the invention provides a method of overcoming resistance to chemotherapeutic agents in tumor cells, comprising administering an antagonist or inhibitor to the SHH/GLI signaling pathway to the cell, wherein said administering results in increased sensitivity of the tumor cell to said chemotherapeutic agent and results in subsequent tumor cell death and prevention from metastasis, and wherein said antagonist or inhibitor is selected from the group consisting of a small molecule inhibitor or antagonist of GLI synthesis and/or expression and/or activity, an antisense molecule or a siRNA molecule that is essentially complementary to at least a part of the sequence of a nucleic acid encoding the GLI protein, antibodies to GLI, and agents that exhibit mimicry to GLI or antagonism to it or control over its production.

In one particular embodiment, the antisense molecule comprises a nucleic acid sequence that is complementary to the nucleic acid that encodes at least one GLI protein. In another particular embodiment, the antisense molecule comprises the nucleic acid sequence as set forth in SEQ ID NO: 33. In another particular embodiment, the siRNA molecule is selected from the group consisting of the nucleic acid sequences of SEQ ID NO: 29, SEQ ID NO: 31 and SEQ ID NO: 32. In another particular embodiment, the small molecule inhibitor of GLI synthesis and/or expression and/or activity is cyclopamine, jervine and/or analogs or derivatives thereof.

A fifth aspect of the invention provides a method for inhibiting and/or attenuating the expression of one or several gli genes in a mammalian cell, said method comprising:
a) introducing into the cell a nucleic acid molecule having a nucleotide sequence which is substantially identical to or complementary to at least a part of the gli gene in an amount sufficient to inhibit and/or attenuate expression of a gli gene;
b) verifying inhibition and/or attenuation of expression of the gli gene; and
   wherein the mammalian cell is a tumor cell or a cell obtained from tissue exhibiting hyperplasia that is characterized by the expression of at least one gli gene or gene product.

In one particular embodiment, the gli gene is selected from the group consisting of gli1, gli2 and gli3. In another particular embodiment, the nucleic acid molecule is RNA or DNA. In another particular embodiment the RNA is a single stranded RNA (SS-RNA), or a double stranded RNA (DS-RNA) molecule. In another particular embodiment the DNA is a DNA antisense oligonucleotide. In another particular embodiment, the SS-RNA molecule is an antisense RNA molecule. In yet another particular embodiment, the SS-RNA molecule, when administered to a mammal having a tumor cell characterized by the presence of said gli gene, results in inhibition of tumor cell proliferation. In another particular embodiment, the DS-RNA is a siRNA molecule. In yet another particular embodiment, the siRNA molecule, when administered to a mammal having a tumor cell characterized by the presence of said gli gene, results in inhibition of tumor cell proliferation. In another particular embodiment, the siRNA hybridizes under stringent conditions to the gli gene. In another particular embodiment, the siRNA molecule is about 10-50 nucleotides in length. In another particular embodiment, the siRNA molecule is about 10-30 nucleotides in length. In another particular embodiment, the siRNA molecule is about 17-25 nucleotides in length. In another particular embodiment, the siRNA molecule is selected from the group consisting of the nucleic acid sequences set forth in SEQ ID NO: 29, SEQ ID NO: 31 and SEQ ID NO: 33.

A sixth aspect of the invention provides a double stranded siRNA for inhibiting expression of a gli gene. In one embodiment, the siRNA comprises a first nucleotide sequence that hybridizes under stringent conditions to a nucleotide sequence of a gli gene and/or its mRNA and a second nucleotide sequence that is complementary to said first nucleotide sequence. In a particular embodiment, the siRNA is about 10-50 nucleotides long. In another particular embodiment, the siRNA is about 10-50 nucleotides long. In another particular embodiment, the siRNA is about 10-30 nucleotides long. In yet another particular embodiment, the siRNA is about 17-25 nucleotides long. In a another particular embodiment, the siRNA is selected from the group consisting of the nucleic acid sequences as set forth in SEQ ID NO: 29, SEQ ID NO: 31 and SEQ ID NO: 32. In yet another particular embodiment, the siRNA, upon delivery to a cell containing the gli gene for which said siRNA molecules are specific, inhibits the proliferation of said cell. In yet another particular embodiment, the double stranded siRNA is a short hairpin RNA (shRNA) comprising a first nucleotide sequence that hybridizes under stringent conditions to a nucleotide sequence of a gli gene, and a second nucleotide sequence which is a complementary inverted repeat of said first nucleotide sequence and hybridizes to said first nucleotide sequence to form a hairpin structure. In another particular embodiment, the shRNA may have a foreign hairpin from another gene and the stem of the hairpin is formed by the gli sequences.

A seventh aspect of the invention provides a pharmaceutical composition comprising at least one inhibitor or antagonist of GLI synthesis and/or expression and/or activity and a pharmaceutically acceptable carrier. In one particular embodiment, the inhibitor or antagonist is selected from the group consisting of a small molecule inhibitor or antagonist of GLI synthesis and/or expression and/or activity, an antisense molecule or a siRNA molecule that is essentially complementary to at least a part of the sequence of a nucleic acid encoding the GLI protein, antibodies to GLI, and agents that exhibit mimicry to GLI or antagonism to it or control over its production, said contacting or introducing resulting in inhibition of synthesis and/or expression and/or activity of the GLI protein in said cell. In another particular embodiment, the pharmaceutical composition comprises an antisense molecule that comprises the nucleic acid sequence as set forth in SEQ ID NO: 33. In a yet further embodiment, the pharmaceutical composition comprises a siRNA molecule that comprises the nucleic acid sequence as set forth in SEQ ID NOs: 29, 31 and 32. In a yet further embodiment, the pharmaceutical composition comprises a small molecule inhibitor comprising a plant alkaloid, such as, but not limited to cyclopamine, jervine and/or analogs or derivatives thereof.

An eighth aspect of the invention provides methods for screening to identify a subject that is likely to be suffering from a hyperproliferative condition or a cancerous condition. In one particular embodiment, the method comprises:

determining the level of at least one GLI protein or a nucleic acid encoding at least one GLI protein in a tissue specimen or in a specimen of bodily fluid from said subject; wherein when the level of GLI protein or a nucleic acid encoding said GLI protein determined is significantly elevated in cells obtained from said tissue or bodily fluid compared to a predetermined range of normal values established from screening individuals known to be free of a hyperproliferative or cancerous condition, the animal subject is identified as being likely to have a hyperproliferative condition or a cancerous condition.

In another particular embodiment, the method for determining whether a subject that is likely to be suffering from a hyperproliferative condition or a cancerous condition is performed in situ or in vitro. In yet another particular embodiment, the determining is performed by a method that uses a probe selected from the group consisting of an antibody specific for at least one GLI protein; a set of primers specific for an mRNA encoding said GLI protein; and a nucleotide probe specific for an mRNA encoding said GLI protein. In yet another particular embodiment, the method for determining comprises analysis of bodily fluid taken from said subject, wherein said bodily fluid is selected from the group consisting of whole blood, serum, plasma, urine and cerebrospinal fluid.

In a yet further embodiment, the determination of the level of GLI is performed with an antibody specific for GLI. In a yet further embodiment, the level of GLI is determined using an antibody specific for GLI for Western blot analysis. In another such embodiment the determination of the level of GLI is performed by PCR, including real-time PCR, with a primer/primers specific for an mRNA encoding GLI. In still another embodiment the determination of the level of GLI is performed with a nucleotide probe specific for an mRNA encoding GLI. In one such embodiment, the determination of the level of GLI is performed by a Northern blot. In another embodiment, the determination of the level of GLI is performed by a ribonuclease protection assay. In yet another embodiment, the level of GLI is determined using standard immunoassay procedures, such as enzyme linked immunoassay (ELISA) or radioimmunoassay (RIA). In another embodiment, the level of GLI is determined using reporter assays wherein the reporter is a dihydrofolate reductase, a lactamase, a luciferase, a green fluorescent protein, or a yellow fluorescent protein.

A ninth aspect of the invention provides a method of predicting resistance of a tumor cell to chemotherapeutic agents comprising measuring the level of expression of GLI in said tumor cell, whereby an increased level of expression of GLI in said tumor cell correlates with resistance to a chemotherapeutic agent in said tumor cell and is predictive of resistance of said tumor cell to chemotherapeutic agents. In a particular embodiment, the method of predicting resistance to a chemotherapeutic agent may correlate with an increase in level of any one of the gli genes or gene products. In a yet further particular embodiment, the gli gene or gene product (such as the expressed protein) may be selected from any of the GLI proteins, including GLI1, GLI2 and/or GLI3. In another particular embodiment, the tumor cells for which resistance to a chemotherapeutic agent correlates with an increase in level of GLI are selected from the group consisting of gliomas, medulloblastomas, primitive neuroectodermal tumors (PNETS), basal cell carcinoma, small cell lung cancers, large cell lung cancers, tumors of the gastrointestinal tract, rhabdomyosarcomas, soft tissue sarcomas, pancreatic tumors, bladder tumors and prostate tumors. Standard immunological or molecular biological techniques may be used to predict resistance of a tumor cell to chemotherapeutic agents.

A tenth aspect of the invention provides a method of screening for necrotic, senescent, or apoptotic resistant tumor cells, comprising measuring the level of expression of GLI in said tumor cells, wherein enhanced expression of GLI correlates with apoptosis resistance in said tumor cells. In a particular embodiment, the tumor cells are selected from the group consisting of gliomas, medulloblastomas, primitive neuroectodermal tumors (PNETS), basal cell carcinoma, small cell lung cancers, large cell lung cancers, tumors of the gastrointestinal tract, rhabdomyosarcomas, soft tissue sarcomas, pancreatic tumors, bladder tumors and prostate tumors. In another particular embodiment, the screening may be done by immunological methods, such as western blotting techniques, immunofluorescence or radioimmunoassays using an antibody specific for GLI. Alternatively, standard molecular biological approaches may be used, including PCR techniques using primers or probes specific for GLI.

An eleventh aspect of the invention provides for detection of GLI in prostate samples, or in/from metastatic cells present in bodily fluids from a subject as a means of correlating the presence of a gli gene or gene product, such as a GLI protein, with the presence of prostatic cancer. If GLI is present in a prostate sample from the subject, using the procedures outlined below which are known to one skilled in the art, then there is an increased risk of the presence of a prostate tumor. GLI, if present in low amounts, can be detected by PCR, Western blot and/or Northern blot and/or as provided for herein. When GLI is not measurable at all in the prostate sample from the animal subject, the animal subject is identified as being unlikely to have a prostate cancer. In one embodiment, the sample is obtained by radical prostatectomy. In another embodiment, the sample is obtained by needle biopsy. In yet another embodiment, the level of GLI is determined in a bodily sample selected from whole blood, plasma, serum, urine or cerebrospinal fluid.

In one embodiment the determination of the level of GLI is performed in situ. In another embodiment the determination of the level of GLI is performed in vitro. In still another embodiment, the determination of the level of GLI is performed in vivo. In a particular embodiment the determination of the level of GLI is performed with an antibody specific for GLI. In another such embodiment the determination of the level of GLI is performed by PCR with a primer specific for an mRNA encoding GLI. In still another embodiment the determination of the level of GLI is performed with a nucleotide probe specific for an mRNA encoding GLI. In still another embodiment the determination of the level of GLI is performed by a Northern blot. In yet another embodiment the determination of the level of GLI is performed by a ribonuclease protection assay. In still another embodiment the determination of the level of GLI is performed by immunohistochemistry. In still another embodiment the determination of the level of GLI is performed by RT-PCR.

A twelfth aspect of the invention provides for the use of GLI as a biomarker for prostate cancer as well as other cancers which utilize the SHH/GLI pathway for cell signaling, and in which GLI is present. In one embodiment, the presence of increasing levels of the gli gene or gene product, such as a GLI protein, is indicative of an increased risk for the presence of a cancer. In a preferred embodiment, the metastasis is to bone and to other organs.

A thirteenth aspect of the invention provides an isolated nucleic acid probe specific for GLI. In a particular embodiment, the probe is used to identify GLI in a tumor sample. In another particular embodiment, the probe can differentiate between the presence and absence of tumors, such as, but not limited to, prostate tumors and benign tumors in a patient sample. In another particular embodiment, the GLI is a mammalian GLI. In yet another particular embodiment, the mammalian GLI is human GLI. In yet another particular embodiment, the GLI is GLI1, GLI2 or GLI3.

A fourteenth aspect of the invention provides a recombinant DNA molecule that is operatively linked to an expression control sequence; wherein the recombinant DNA molecule comprises a nucleic acid that encodes human GLI.

A fifteenth aspect of the invention provides an expression vector containing the recombinant DNA molecule encoding GLI.

A sixteenth aspect of the invention provides a cell that has been transformed with the expression vector containing the recombinant DNA molecule encoding GLI, wherein said GLI protein is expressed by the cell. In a particular embodiment, the cell is a mammalian cell. The invention further provides a method of expressing a recombinant GLI protein in a cell containing the expression vector comprising culturing the cell in an appropriate cell culture medium under conditions that provide for expression of recombinant GLI by the cell.

Other aspects and advantages will become apparent from a review of the ensuing detailed description taken in conjunction with the following illustrative drawings.

### BRIEF DESCRIPTION OF THE DRAWINGS

FIG. 1 SSH-GLI pathway and potential sites for therapeutic agents blocking its activity

FIG. 2 Demonstration that GLI genes are consistently expressed in primary brain tumors

FIG. 3 Demonstration that Cyclopamine, a drug that inhibits the response to Shh signaling modulates the proliferation of a subset of brain tumor cell lines

FIG. 4A, B, C Effect of cyclopamine in a long-term treatment of a glioblastoma cell line (U87) in vitro. Shh-Gli pathway controls proliferation and viability of brain tumor cells

FIG. 5 Cyclopamine modulates the proliferation of primary cortical gliomas that were dissociated and cultured in vitro

FIG. 6A,B Demonstration that only a subset of cells from primary brain tumors, dissociated and cultured in vitro, have stem-like properties, and their proliferation is inhibited by the presence of cyclopamine

FIG. 7A, B, C In vivo cyclopamine treatment reduces the size of medulloblastomas of Ptch+/−, p53−/− mice

FIG. 8 Percentage of BrdU incorporation in the presence or absence of 5 uM cyclopamine

FIG. 9 siRNAs for Gli1 and Gli2 block Shh responses in 10T1/2 cells

FIG. 10 Downregulation of Gli1 and Gli2 inhibits U87 glioma cell proliferation

FIG. 11 Proliferation of primary brain tumor cells is inhibited by blocking Gli1 and Gli2

FIG. 12 Expression of SHH-GLI pathway components in normal prostate tissue and prostate tumors. A-Q) Sections of normal prostate tissue (A, C, E, G, I, L, O) and prostate tumors (B, D, F, H, J, K, M, N, P, Q) showing hematoxylin and eosin staining (H&E, A, B) or the expression of SHH(C, D, I-K), PTCH1 (E, F, L-N) and GLI1 (G, H, O-Q). Inset in G) sense GLI1 probe control showing no background. Prostate tumors have many small epithelial glandular structures. Black arrows point of expressing cells. White arrows point to non-expressing cells. R-T) Sections from the tissue microarrays of normal prostate tissue (R) and prostate tumors (S,T) showing expression of SHH protein with an anti-SHH antibody (αSHH Ab) (R-T) and a no-primary antibody control (inset in T). All sections were counterstained with hematoxylin to visualize nuclei and tissue structure. Arrow in (T) points to localization of SHH protein in the cytoplasm of epithelial cells. e: epithelium, l:lumen, s:stroma, t:tumor. Scale bar in (T)=150 μm for (A-H, R, S), 20 μm for (J, M, P, T) and 10 μm for (I, L, O, K, N, Q). Histogram error bars in all figures represent s.e.m.

FIG. 13 Response of prostate tumor cell lines to alterations in the SHH-GLI pathway. a) PCR analyses for the expression of SHH-GLI pathway components in three cell lines as indicated. In this and all other PCR assays the expression of the ubiquitous gene GAPDH is measured as quantitative control. b) Inhibition of prostate cell line proliferation as measured by BrdU incorporation in the three prostate cell lines used with cyclopamine. Tomatidine is used as control. c,d) PCR analyses of the suppression of GLI1 expression in LNCaP cells by cyclopamine treatment at 36 h (c) or of Prostate specific antigen (PSA) expression in whole tissue (T), primary culture (C), the glioblastoma cell line U87 (U) and LNCaP (L) cells. PSA is expressed in prostate but not in brain cells. The whole tissue and primary culture correspond to PT6. e) Histogram of the inhibition of BrdU incorporation in primary cultures of prostate tumor (PT3-PT8) by cyclopamine treatment. f-i) Immunocytochemistry for BrdU incorporation with secondary FITC-antibodies showing BrdU⁺ nuclei (green) in a field of primary prostate cells (PT6) in control cells (treated with ethanol as the carrier for cyclopamine, f), cyclopamine (g), SHH protein (h) or anti-SHH antibody (αSHH Ab, i). All nuclei are stained with DAPI (blue). j,k) Histograms of the increase in (j) or inhibition of (k) BrdU incorporation of primary prostate tumors after treatment with SHH (j) or anti-SHH antibody (αSHH Ab, k) for 48 h.

FIG. 14 Response of prostate cell lines to GLI1 RNA interference. A-C) Immunocytochemisty of the three prostate cell lines indicated showing the efficiency of lipofection of an FITC-tagged control siRNA (green). Note the lower efficiency in PC3 cells. D) Effect of Gill siRNA on gene expression. RNA interference reduces GLI1 and PTCH1 mRNA levels as seen at 2 h and 48 h, respectively E) Histogram of the inhibition of BrdU incorporation in prostate tumor cell lines by GLI1 siRNA. F) Specificity of the effects of GLI1 siRNA on GLI1 mRNA levels in the three prostate cell lines, compared with those of a control unrelated siRNA, after 8 h post-transfection. The levels of GAPDH are shown below as controls.

### DETAILED DESCRIPTION OF THE INVENTION

Before the present methods and treatment methodology are described, it is to be understood that this invention is not limited to particular methods, and experimental conditions described, as such methods and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated by reference in their entireties.

### DEFINITIONS

As noted above, the terms used herein have the meanings recognized and known to those of skill in the art. However, for convenience and completeness, particular terms and their meanings are set forth below.

As used herein the “SHH/GLI pathway” or “HH/GLI pathway” or “GLI-pathway” or “GLI signaling pathway” is used interchangeably with the “Sonic hedgehog (SHE) signaling pathway” and is the signaling pathway initiated by a hedgehog protein binding to its receptor(s) leading to the expression and/or function of a GLI protein. “GLI” as used herein refers to any one of the GLI1, GLI2 or GLI3 proteins. “gli” refers to the gene encoding the GLI proteins, and gli1, gli2 and gli3 are the genes encoding the GLI1, GLI2 and GLI3 proteins. Factors involved and/or can function in the SHH-GLI pathway include any hedgehog protein such as sonic hedgehog, Indian hedgehog, and desert hedgehog, patched 1 and 2, smoothened, agonists and antagonists of such proteins, PKA, fused, suppressor of fused, costal-2, and modifiers and/or partners of any of the GLI1, 2, or 3 proteins e.g., the Zic gene products.

As used herein the term “hedgehog” is used interchangeably with the term “HH” and is a cytokine that binds to the HH receptor to stimulate the beginning of the SHH-GLI pathway. The human SHH protein is encoded by the nucleotide sequence of SEQ ID NO:1 and has the amino acid sequence of SEQ ID NO:2. The murine SHH protein is encoded by the nucleotide sequence of SEQ ID NO:3 and has the amino acid sequence of SEQ ID NO:4. The rat SHH protein is encoded by the nucleotide sequence of SEQ ID NO:5 and has the amino acid sequence of SEQ ID NO:6. Xenopus HH protein is encoded by the nucleotide sequence of SEQ ID NO:7 and has the amino acid sequence of SEQ ID NO:8. The human Indian hedgehog (IHH) protein is encoded by the nucleotide sequences of SEQ ID NO:9 and/or 11 and has the amino acid sequence of SEQ ID NO:10 and/or 12. The murine desert hedgehog (DHH) protein is encoded by the nucleotide sequence of SEQ ID NO:13 and has the amino acid sequence of SEQ ID NO:14. Hedgehog proteins from species as different as humans and insects appear to play this same role and can be used interchangeably (see e.g., Pathi et al., (2001) Mech Dev. 106:107-117).

As used herein an “active fragment” of a hedgehog is a fragment of a hedgehog protein that can comprises the first 174 amino acids of the protein (not counting the signal sequence) and can stimulate both the in vitro proliferation and in vitro differentiation of a mouse subventricular stem cell such that the number of neuronal cells obtained in the presence of 100 μM or less active fragment of the HH is at least 2-fold greater than that obtained in its absence.

As used herein a “small organic molecule” is an organic compound (or organic compound complexed with an inorganic compound (e.g., metal)) that has a molecular weight of less than 3 kilodaltons, and preferably less than 1.5 kilodaltons.

As used herein a “reporter” gene is used interchangeably with the term “marker gene” and is a nucleic acid that is readily detectable and/or encodes a gene product that is readily detectable such as green fluorescent protein (as described in U.S. Pat. No. 5,625,048 issued Apr. 29, 1997, and WO 97/26333, published Jul. 24, 1997, the disclosures of each are hereby incorporated by reference herein in their entireties) or luciferase.

A “vector” is a replicon, such as plasmid, phage, virus, or cosmid, to which another DNA segment may be attached so as to bring about the replication of the attached segment. A “replicon” is any genetic element (e.g., plasmid, chromosome, virus) that functions as an autonomous unit of DNA replication in vivo, i.e., capable of replication under its own control.

Transcriptional and translational control sequences are DNA regulatory sequences, such as promoters, enhancers, terminators, and the like, that provide for the expression of a coding sequence in a host cell. In eukaryotic cells, polyadenylation signals are control sequences.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” of transcriptional and translational control sequences in a cell when RNA polymerase transcribes the coding sequence into mRNA, which is then trans-RNA spliced and translated into the protein encoded by the coding sequence.

As used herein, the term “homologue” is used interchangeably with the term “ortholog” and refers to the relationship between proteins that have a common evolutionary origin and differ because they originate from different species. For example, mouse SHH is a homologue of human SHH.

As used herein the term “heterologous nucleotide sequence” is a nucleotide sequence that is added to a nucleotide sequence of the present invention by recombinant molecular biological methods to form a nucleic acid which is not naturally formed in nature. Such nucleic acids can encode chimeric and/or fusion proteins. Thus the heterologous nucleotide sequence can encode peptides and/or proteins which contain regulatory and/or structural properties. In another such embodiment the heterologous nucleotide can encode a protein or peptide that functions as a means of detecting the protein or peptide encoded by a nucleotide sequence of the present invention after the recombinant nucleic acid is expressed. In still another embodiment the heterologous nucleotide can function as a means of detecting a nucleotide sequence of the present invention. A heterologous nucleotide sequence can comprise non-coding sequences including restriction sites, regulatory sites, promoters and the like. Thus, the nucleic acids that encode the proteins being used and/or detected in the present invention can comprise a heterologous nucleotide sequence.

As used herein the terms “fusion protein” and “fusion peptide” are used interchangeably and encompass “chimeric proteins and/or chimeric peptides” and fusion “intein proteins/peptides”. A fusion protein of the present invention can comprise at least a portion of a HH protein of the present invention, for example, joined via a peptide bond to at least a portion of another protein or peptide including a second HH protein in a chimeric fusion protein.

As used herein a polypeptide or peptide “consisting essentially of” or that “consists essentially of” a specified amino acid sequence is a polypeptide or peptide that retains the general characteristics, e.g., activity of the polypeptide or peptide having the specified amino acid sequence and is otherwise identical to that protein in amino acid sequence except it consists of plus or minus 10% or fewer, preferably plus or minus 5% or fewer, and more preferably plus or minus 2.5% or fewer amino acid residues.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

The phrase “therapeutically effective amount” is used herein to mean an amount sufficient to reduce by at least about 15 percent, preferably by at least 50 percent, more preferably by at least 90 percent, and most preferably prevent, a clinically significant deficit in the activity, function and response of the host. Alternatively, a therapeutically effective amount is sufficient to cause an improvement in a clinically significant condition/symptom in the host.

In a specific embodiment, the term “about” means within 20%, preferably within 10%, and more preferably within 5%.

RNA interference (RNAi) is an evolutionarily conserved mechanism in plant and animal cells that directs the degradation of messenger RNAs homologous to short double-stranded RNAs termed “small interfering RNA (siRNA)”. The ability of siRNA to direct gene silencing in mammalian cells has raised the possibility that siRNA might be used to investigate gene function in a high throughput fashion or to modulate gene expression in human diseases. Methods of preparing siRNAs are known to those skilled in the art. The following references are incorporated herein by reference in their entirety: Reich et al., Mol. Vis. 9:210-6 (2003); Gonzalez-Alegre P et al., Ann Neurol. 53:781-7 (2003); Miller et al., Proc Natl Acad Sci USA. (2003); Bidere et al., J Biol. Chem., published as manuscript M301911200 (Jun. 2, 2003); Van De Wetering et al., EMBO Rep. 4:609-15 (2003); Miller and Grollman, DNA Repair (Amst) 2:759-63 (2003); Kawakami et al., Nat Cell Biol. 5:513-9 (2003); Abdelrahim et al., Mol. Pharmacol. 63:1373-81 (2003); Williams et al., J. Immunol. 170:5354-8 (2003); Daude et al., J Cell Sci. 116:2775-9 (2003); Jackson et al., Nat. Biotechnol. 21:635-7 (2003); Dillin, Proc Natl Acad Sci USA. 100:6289-91 (2003); Matta et al., Cancer Biol Ther. 2:206-10 (2003); Wohlbold et al., Blood. (2003); Julien and Herr, EMBO J. 22:2360-9 (2003); Scherr et al., Cell Cycle. 2:251-7 (2003); Giri et al., J. Immunol. 170:5281-94 (2003); Liu and Erikson, Proc Natl Acad Sci USA. 100:5789-94 (2003); Chi et al., Proc Natl Acad Sci USA. 100:6343-6 (2003); Hall and Alexander, J. Virol. 77:6066-9 (2003).

“Agent” refers to all materials that may be used to prepare pharmaceutical and diagnostic compositions, or that may be compounds, nucleic acids, polypeptides, fragments, isoforms, variants, or other materials that may be used independently for such purposes, all in accordance with the present invention.

“Analog” as used herein, refers to a small organic compound, a nucleotide, a protein, or a polypeptide that possesses similar or identical activity or function(s) as the compound, nucleotide, protein or polypeptide or compound having the desired activity and therapeutic effect of the present invention. (eg. inhibition of tumor growth), but need not necessarily comprise a sequence or structure that is similar or identical to the sequence or structure of the preferred embodiment As used herein, a nucleic acid or nucleotide sequence, or an amino acid sequence of a protein or polypeptide is “similar” to that of a nucleic acid, nucleotide or protein or polypeptide having the desired activity if it satisfies at least one of the following criteria: (a) the nucleic acid, nucleotide, protein or polypeptide has a sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the nucleic acid, nucleotide, protein or polypeptide sequences having the desired activity as described herein (b) the polypeptide is encoded by a nucleotide sequence that hybridizes under stringent conditions to a nucleotide sequence encoding at least 5 amino acid residues (more preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues) of the AAPI; or (c) the polypeptide is encoded by a nucleotide sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the nucleotide sequence encoding the polypeptides of the present invention having the desired therapeutic effect. As used herein, a polypeptide with “similar structure” to that of the preferred embodiments of the invention refers to a polypeptide that has a similar secondary, tertiary or quarternary structure as that of the preferred embodiment. The structure of a polypeptide can determined by methods known to those skilled in the art, including but not limited to, X-ray crystallography, nuclear magnetic resonance, and crystallographic electron microscopy.

The term “antibody” as used herein includes intact molecules as well as fragments thereof, such as Fab and F(ab′)₂, which are capable of binding the epitopic determinant. Antibodies that bind the genes or gene products of the present invention can be prepared using intact polynucleotides or polypeptides or fragments containing small peptides of interest as the immunizing antigen attached to a carrier molecule. Commonly used carriers that are chemically coupled to peptides include bovine serum albumin and thyroglobulin. The coupled peptide is then used to immunize the animal (e.g, a mouse, rat or rabbit). The antibody may be a “chimeric antibody”, which refers to a molecule in which different portions are derived from different animal species, such as those having a human immunoglobulin constant region and a variable region derived from a murine mAb. (See, e.g., Cabilly et al., U.S. Pat. No. 4,816,567; and Boss et al., U.S. Pat. No. 4,816,397.). The antibody may be a single chain antibody. The antibody may be a human or a humanized antibody. The antibody may be prepared in mice, rats, goats, sheep, swine, dogs, cats, or horses. A “blocking antibody” refers to an antibody that interferes with the function, activity or expression of a particular molecule, in the matter of the present invention, an antibody to SHH or GLI.

“Apoptosis” refers to programmed cell death and is characterized by certain cellular characteristics such as membrane blebbing, chromatin condensation and fragmentation, formation of apoptotic bodies and a positive “TUNEL” staining pattern. Degradation of genomic DNA during apoptosis results in formation of characteristic, nucleosome sized DNA fragments; this degradation produces a diagnostic (about) 180 bp laddering pattern when analyzed by gel electrophoresis. A later step in the apoptotic process is degradation of the plasma membrane, rendering apoptotic cells leaky to various dyes (e.g., trypan blue and propidium iodide).

“Surrogate biomarker” or “biomarker” as used herein, refers to a highly specific molecule, the existence and levels of which are causally connected to a complex biological process, and reliably captures the state of said process. Furthermore, a surrogate biomarker, to be of practical importance, must be present in samples that can be obtained from individuals without endangering their physical integrity or well-being, preferentially from biological fluids such as blood, urine, saliva or tears.

“Derivative” refers to either a compound, a protein or polypeptide that comprises an amino acid sequence of a parent protein or polypeptide that has been altered by the introduction of amino acid residue substitutions, deletions or additions, or a nucleic acid or nucleotide that has been modified by either introduction of nucleotide substitutions or deletions, additions or mutations. The derivative nucleic acid, nucleotide, protein or polypeptide possesses a similar or identical function as the parent polypeptide.

“Fragment” refers to either a protein or polypeptide comprising an amino acid sequence of at least 5 amino acid residues (preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, at least 150 amino acid residues, at least 175 amino acid residues, at least 200 amino acid residues, or at least 250 amino acid residues) of the amino acid sequence of a parent protein or polypeptide, or a nucleic acid comprising a nucleotide sequence of at least 10 base pairs (preferably at least 20 base pairs, at least 30 base pairs, at least 40 base pairs, at least 50 base pairs, at least 50 base pairs, at least 100 base pairs, at least 200 base pairs) of the nucleotide sequence of the parent nucleic acid. Any given fragment may or may not possess a functional activity of the parent nucleic acid or protein or polypeptide.

“Treatment” or “treating” refers to therapy, prevention and prophylaxis and particularly refers to the administration of medicine or the performance of medical procedures with respect to a patient, for either prophylaxis (prevention) or to cure or reduce the extent of or likelihood of occurrence of the infirmity or malady or condition or event in the instance where the patient is afflicted.

“Diagnosis” refers to diagnosis, prognosis, monitoring, characterizing, selecting patients, including participants in clinical trials, and identifying patients at risk for or having a particular disorder or clinical event or those most likely to respond to a particular therapeutic treatment, or for assessing or monitoring a patient's response to a particular therapeutic treatment.

“Subject” or “patient” refers to a mammal, preferably a human, in need of treatment for a condition, disorder or disease.

“Inhibitors,” or “antagonists” refer to inhibitory molecules identified using in vitro and in vivo assays for SHH-GLI pathway function. In particular, inhibitors and antagonists refer to compounds or agents that decrease signaling that occurs via the SHH-GLI pathway. Inhibitors may be compounds that decrease, block, or prevent, signaling via this pathway. Such assays for inhibitors or antagonists may include assaying for the effect of such compounds on neurogenesis or on tumor cell proliferation or inhibition thereof.

“Antisense” nucleic acids are DNA or RNA molecules that are complementary to at least a portion of a specific mRNA molecule (See Weintraub, Sci. Amer. 262:40-46 (1990); Marcus-Sekura, Nucl. Acid Res, 15: 5749-5763 (1987); Marcus-Sekura Anal. Biochem., 172:289-295 (1988); Brysch et al., Cell Mol. Neurobiol., 14:557-568 (1994)). In the cell, the single stranded antisense molecule hybridizes to that mRNA, forming a double stranded molecule. The cell does not translate an mRNA in this double-stranded form. Therefore, antisense nucleic acids interfere with the expression of mRNA into protein. Oligomers of greater than about fifteen nucleotides and molecules that hybridize to the AUG initiation codon will be particularly efficient. Antisense methods have been used to inhibit the expression of many genes in vitro (Marcus-Sekura, Anal. Biochem., 172:289-295 (1988); Hambor et al., Proc. Natl. Acad. Sci. U.S.A. 85:4010-4014 (1988)) and in situ (Arima et al., Antisense Nucl. Acid Drug Dev. 8:319-327 (1998); Hou et al., Antisense Nucl. Acid Drug Dev. 8:295-308 (1998)).

The term “oligonucleotide,” as used herein is defined as a molecule comprised of two or more ribonucleotides, preferably more than three. Its exact size will depend upon many factors which, in turn, depend upon the ultimate function and use of the oligonucleotide.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The term “standard hybridization conditions” refers to salt and temperature conditions substantially equivalent to 5×SSC and 65° C. for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also important in the determination of “standard hybridization conditions” is whether the two sequences hybridizing are RNA-RNA, DNA-DNA or RNA-DNA. Such standard hybridization conditions are easily determined by one skilled in the art according to well known formulae, wherein hybridization is typically 10-20° C. below the predicted or determined Tₘ with washes of higher stringency, if desired.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

Agents that “increase negative, or decrease positive acting elements affecting SHH-GLI signaling”, as used herein, refers to agents that potentiate positive or transactivating function of the Gli proteins, for example, increasing their transcriptional activity on target genes, or agents that decrease their negative or repressive transcription function on target genes.

Tumors that are “apoptotic resistant” are tumors that are inhibited from undergoing apoptosis. This inhibition of apoptosis may be due in part to the expression of at least one of the GLI proteins or gene products.

Tumors that are “resistant to chemotherapeutic agents”, are tumors that do not respond to treatment with chemotherapeutic agents, ie. they are not killed by, or their growth is not inhibited by, such treatment. Such resistance may be due at least in part to the expression of at least one of the GLI proteins or gene products.

### General Description

The Hedgehog-Gli signaling pathway, or SHH/GLI signaling pathway, regulates numerous events during the normal development of many cell types and organs, including the brain, bone, skin, gonads, lung, prostate, gastrointestinal tract and blood. The hedgehog (hh) gene—like many of the components of the signaling pathway triggered by Hedgehog (Hh) protein—was first identified in Drosophila, where it affects pattern formation very early in embryonic development. The binding of Hh to cell membranes triggers a signaling cascade that results in the regulation of transcription by zinc-finger transcription factors of the Gli family.

Of the three hh-family genes in mammals—Sonic hedgehog (Shh), Indian hedgehog (Ihh) and Desert hedgehog (Dhh)—Shh has been the most studied, mainly because it is expressed in various tissues but also because experiments with Shh protein are generally also applicable to other members of the family. The correct regulation of the Hh-Gli signaling pathway is essential not only for normal development but also to prevent a number of human diseases associated with abnormally increased or decreased signaling. Here, we discuss the potential use of small-molecule modulators of the Hh-signaling system.

Hedgehogs are secreted glycoproteins that act through the transmembrane proteins Patched1 (Ptc1) and Smoothened (Smo) to activate an intricate intracellular signal-transduction pathway. Hh binds Ptc1, a protein with 12 trans-membrane domains, and this releases the basal repression that Ptc1 exerts on Smo, a 7-transmembrane-domain protein that has homology to G-protein-coupled receptors. Inside the cell, a multimolecular complex, including Costal2 (Cos2), Fused (Fu) and suppressor of Fused (Su(Fu)), responds to the activation of Smo in such a way as to modify the activity of the Gli proteins. There are three Gli transcription factors in vertebrates: Gli1 appears to act as a transcriptional activator and is universally induced in Hh-responding cells, whereas Gli2 and Gli3 can act as activators or repressors of transcription depending on the particular cellular context. The fate of Gli proteins, which appear to reside in the cytoplasm in their inactive state, depends on the state of Hh signaling. In the absence of Hh, Gli 3 is processed into a smaller, nuclear transcriptional repressor that lacks the carboxy-terminal domain of full-length Gli3. Upon activation of Smo (and Hh signaling), Gli3 protein cleavage is prevented and an apparent full-length form with transcription-activating function is generated. Gli2 also encodes a repressor function in its carboxy-terminally truncated form, but its formation does not appear to be regulated by Hh signaling.

Mutations in components of the HH-GLI pathway in humans (human gene and protein names are given in capitals) lead to several diseases that result from either loss of function or ectopic activation of the pathway. For example, haploinsufficiency of SHH or mutation in the human PTCH1 gene are associated with holoprosencephaly, a common syndrome affecting development of the forebrain and mid-face. Moreover, ectopic expression of Shh, Gli1 or Gli2 in model systems leads to the formation of tumors that resemble basal cell carcinomas (BCCs), and sporadic human BCCs consistently express GLI, suggesting that all sporadic BCCs have this pathway active. Similarly, human mutations in the Suppressor of Fused—SU(FU)-gene predispose the carrier to medulloblastoma; sporadic medulloblastomas can carry PTCH1 mutations and express GLI1—again suggesting that they harbor an active pathway—and Ptc+/− mice can develop medulloblastomas. Furthermore, a number of sporadic prostate tumors express GLI1 (Dahmane N, et al. The Sonic Hedgehog-Gli pathway regulates dorsal brain growth and tumorigenesis. Development 128, 5201-5212 (2001)), a reliable marker of HH signaling (Lee J, Platt K A, Censullo P, & Ruiz i Altaba A. Gli1 is a target of Sonic hedgehog that induces ventral neural tube development. Development 124, 2537-2552 (1997), raising the possibility that this pathway participates in prostate cancer (PC). Moreover, genetic mapping data reveals that at least two of the genes present in the SHH-GLI pathway (SMOH and Suppressor of Fused (SUFUH)) are located in chromosomal regions implicated in familial human PC by genetic mapping studies Easton, D. F., Schaid, D. J., Whittemore, A. S. & Isaacs, W. J., Prostate 57, 261-269 (2003); Xu, J. et al., Prostate 57, 320-325 (2003).

From an examination of the different mutations that cause aberrant suppression or activation of the HH-GLI pathway in humans, it seems clear that the development of small molecules that could act as agonists or antagonists of the function of proteins such as PTCH1, SMO or GLI might provide an effective therapeutic approach. One such drug could be SHH protein itself, a natural agonist. For example, it has been reported that injection of Shh into the striatum reduces behavioral deficits in a rat model of Parkinson's disease, that Shh can induce dopaminergic neuronal differentiation and that Shh is a neuroprotective agent. But Shh has a relatively short half-life in serum and its therapeutic effects have been difficult to evaluate in vivo. The use of synthetic Hh agonists could therefore provide a viable alternative to Shh protein. Frank-Kamenetsky et al. have now identified a synthetic non-peptidyl small molecule that faithfully activates the Hh-Gli pathway, triggering the known biological effects of Hh signaling. They have shown that this agonist promotes proliferation and differentiation in a cell-type-specific manner in vitro, while in vivo it rescues developmental defects of Shh-null mouse embryos. But this agonist, unlike Shh protein, appears to bypass the Ptc1-regulatory step, by interacting directly with Smo (Journal of Biology 2002, Volume 1, Issue 2, Article 9; Stecca and Ruiz i Altaba, Journal of Biology 2002, 1:9. From a therapeutic point of view, the fact that the molecule retains its activity after oral administration is a great advantage and, if its ability to cross the blood-brain and placental barriers occurs in humans, it could be a very valuable therapeutic agent. Nevertheless, systemic side effects are to be expected, as there are many HH-responsive cell populations in the body.

Treatment of human diseases resulting from ectopic HH-GLI pathway activation, such as those caused by oncogenic mutations in SMOH and PTCH1 or in any element of the pathway that results in activation of GLI function, requires the use of pathway antagonists. Up to now, inhibition of ectopic activity has been achieved by treatment with signaling antagonists that block the pathway at different levels: first, blocking anti-Shh antibodies that act extracellularly, second, cyclopamine, a plant alkaloid that acts at the level of Smo in the cell membrane, third, forskolin, an intracellular activator of protein kinase A (PKA) that is a cytoplasmic inhibitor of the pathway; and fourth, Gli-repressor proteins that act within the nucleus to inhibit positive GLI function from mediating the HH signal. Use of forskolin is likely to lead to numerous side effects, given the wide-spread activity of PKA. In contrast, the use of the small molecule cyclopamine or analogs thereof, holds great promise.

A number of studies suggest that cyclopamine specifically inhibits Smo activity and that it can affect disease states caused by activation of the HH-GLI pathway. For example, the proliferation of a number of human brain-tumor cell lines and primary tumor cultures, including those from medulloblastomas and some gliomas as well as medulloblastoma allografts, are inhibited by treatment with cyclopamine. This suggests that pathway activation is required for tumor maintenance. The activity of Gli proteins, the terminal elements of the pathway, is sufficient to induce tumor development. Thus, HH-pathway activity may be involved in the initiation as well as the maintenance of different tumors. This provides an additional opportunity to inhibit the growth of a number of tumors in different organs and tissues, including basal cell carcinoma in the skin, lung cancer (small cell and non small cell lung cancer), prostate cancer, medulloblastoma, glioblastoma and PNETs, and other brain tumors, cancer of the stomach, GI tract, pancreas, rhabdomyosarcomas, and soft tissue sarcomas, all with the same agent. Cyclopamine could be such an agent if the diseases to be treated arise from activation of the HH-signaling pathway at the level of SMOH or above. But cyclopamine is currently very expensive, and alternative HH-pathway antagonists might be economically more attractive. Frank-Kamenetsky et al.(Frank-Kamenetsky et al J. Biol. 2002, 1:10) report the use of a new, synthetic, small-molecule inhibitor, Cur61414, which has inhibitory properties similar to those of cyclopamine and also acts at the level of Smo (Williams et al, PNAS, (2003), 100(8):4616-21). Whether Cur61414, or four additional small-molecule antagonists (SANT1-4) that also act on Smo and were recently identified (Chen et al, (2002), PNAS, 99:14071-14076), will prove to be better and easier to use than cyclopamine remains to be determined, but testing them against skin (Williams et al supra) and brain tumors is warranted from a biological point of view.

Finally, given that carboxy-terminally truncated repressor forms of GLI3 are potent inhibitors of the activating output of the HH-signaling pathway, these could be used as antagonists for the treatment of tumors. The difficulty of delivering them into cells might require the development of in vivo transducing strategies, taking advantage, for example, of the ability of the Penetratin, TAT or VP22 peptides to cross cell membranes while loaded with cargo. It also suggests that it would be useful to search for and design small molecules that inhibit GLI's transcription-activating function, perhaps by promoting endogenous GLI-repressor formation. This may be very difficult, but such drugs would be very specific and would be usable in cases where the cancer is due to mutation in the pathway at any level, from the extracellular ligand, the HH proteins, to the final mediators, the GLI proteins. Agents that inhibit HH signaling may induce the regression of tumors that are dependent on a deregulated HH-GLI pathway, but these agents are likely also to affect the behavior of other normal pathway-dependent cells in the patient. This may, however, be a small price to pay in order to combat cancer, and the agents may have fewer side effects than current non-specific cytotoxic anti-cancer chemotherapies.

The present invention provides methodology for treating tumors, e.g., treating brain tumors and prostate tumors and other hyperproliferative conditions wherein the condition is characterized as having increased levels of expression of one or more members of the family of GLI proteins. In a preferred embodiment, the invention provides for methods of treating tumors using inhibitors of the sonic hedgehog (SHH) and/or GLI pathways. In another preferred embodiment, the invention provides for treating tumors with antisense RNAs, small interfering RNAs (siRNAs) or cyclopamine. In a yet further embodiment, the tumors are prostate tumors. In yet another particular embodiment, the tumors are inhibited from undergoing apoptosis (they are apoptotic resistant) due in part to the expression of at least one of the GLI proteins or gene products. In another particular embodiment, the tumors are resistant to chemotherapeutic agents, such resistance being due at least in part to the expression of at least one of the GLI proteins or gene products.

A yet further aspect of the invention provides for pharmaceutical compositions comprising the agents that inhibit tumors whose growth is regulated via the SHH and/or GLI pathway and a pharmaceutically acceptable carrier. In particular, the agents that inhibit tumors are selected from the group consisting of a siRNA, antisense nucleic acid, cyclopamine or analogs or derivatives thereof and is formulated in a composition that allows for delivery via an intravenous, intraperitoneal, subcutaneous, intramuscular, intracerebral, intraventricular, or intrathecal route with a pharmaceutically acceptable carrier.

In addition, it has recently been shown that sonic hedgehog is a mitogen for murine cerebellar, neocortical and tectal cell precursors (Dahmane and Ruiz i Altaba, Development 389:3089-3100, 1999). Furthermore, the SHH-GLI pathway has been implicated in the genesis of medulloblastomas (Goodrich et al. Science 277: 1109-1113, 1997), tumors of the cerebellum, suggesting that inappropriate activation or maintenance of this pathway leads to tumorigenesis in this part of the brain. The inventors of the present application provide evidence that the gli genes are expressed in neuronal and glial tumors from different brain locations as well as in established brain tumor cell lines, and that cyclopamine and siRNAs inhibit the proliferation of a high percentage of the primary human gliomas and established cell lines tested. It is thus a further object of the invention to provide for compounds, short or small interfering RNAs, compositions and methods of inhibiting tumorigenesis in a subject in need of such therapy.

### Nucleic Acids Encoding SHH and GLI

The present invention contemplates use of nucleic acids encoding a Hedgehog family member such as sonic hedgehog (e.g., genomic or cDNA) and nucleic acids encoding active fragments thereof, or nucleic acids encoding the GLI family of proteins, including GLI1, GLI2 and GLI3 or active fragments thereof. HH or GLI can be used from any animal species, including insects, but preferably a mammalian source, and more preferably a human source. In accordance with the present invention there may be employed conventional molecular biology, microbiology, and recombinant DNA techniques within the skill of the art. Such techniques are explained fully in the literature. (See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); Sambrook and Russell, Molecular Cloning: A Laboratory Manual, Third Edition (2001) Cold Spring Harbor Laboratory Press, Cold Spring Harbor, N.Y.; DNA Cloning: A Practical Approach, Volumes I and II D. N. Glover ed. 1985; Oligonucleotide Synthesis, M. J. Gait ed. (1984); Nucleic Acid Hybridization, B. D. Hames & S. J. Higgins eds. (1985); Transcription And Translation, B. D. Hames & S. J. Higgins, eds. (1984); Animal Cell Culture. R. I. Freshney, ed. (1986); Immobilized Cells And Enzymes, IRL Press, (1986); B. Perbal, A Practical Guide To Molecular Cloning (1984); F. M. Ausubel et al. (eds.), Current Protocols in Molecular Biology, John Wiley & Sons, Inc. (1994)).

Accordingly, any animal cell potentially can serve as the nucleic acid source for the molecular cloning of an hh gene or a gli gene. The DNA may be obtained by standard procedures known in the art from cloned DNA (e.g., a DNA “library”), by chemical synthesis, by cDNA cloning, or by the cloning of genomic DNA, or fragments thereof, purified from the desired cell (see, for example, Sambrook et al., 1989, supra; Glover, D. M. (ed.), 1985, DNA Cloning: A Practical Approach, MRL Press, Ltd., Oxford, U.K. Vol. I, II). Clones derived from genomic DNA may contain regulatory and intron DNA regions in addition to coding regions; clones derived from cDNA will not contain intron sequences. Whatever the source, the gene should be molecularly cloned into a suitable vector for propagation of the gene.

The nucleotide sequence of the human SHH, SEQ ID NO: 1, or of the human GLI 1, 2, and 3, SEQ ID NOs: 15 (PubMed accession number NM_{—}005269), 17 (PubMed accession number AB007296) and 19 (PubMed accession number NM_{—}000168), respectively, can also be used to search for highly homologous genes from other species, or for proteins having at least one homologous domain, using computer data bases containing either partial or full length nucleic acid sequences. Human ESTs, for example, can be searched. The human Shh sequence can be compared with other human sequences, e.g., in GenBank, using GCG software and the blast search program for example. Matches with highly homologous sequences or portions thereof can then be obtained.

If the sequence identified is an EST, the insert containing the EST can be obtained and then fully sequenced. The resulting sequence can then be used in place of, and/or in conjunction with SEQ ID NO: for SEQ ID NOs: 15, 17 and 19, to identify other ESTs which contain coding regions of the SHH homologue (or SHH domain homologue) or GLI homologue. Plasmids containing the matched EST for example can be digested with restriction enzymes in order to release the cDNA inserts. If the plasmid does not contain the full length homologue the digests can be purified, e.g., run on an agarose gel and the bands corresponding to the inserts can be cut from the gel and purified. Such purified inserts are likely to contain overlapping regions which can be combined as templates of a PCR reaction using primers which are preferably located outside of the SHH open reading frame. Amplification should yield the expected product which can be ligated into a vector and used to transform an E coli derivative e.g., via TA cloning (Invitrogen) for example. A resulting full-length SHH or GLI homologue can be placed into an expression vector and the expressed recombinant SHH or GLI can then be assayed for its ability to stimulate the proliferation and differentiation of brain stem cells. Alternatively, the expressed proteins can be used to prepare antibodies, which can then be used to inhibit the unwanted (tumorigenic) effects of the SHH/GLI pathway.

A modified HH or GLI can be made by altering nucleic acid sequences encoding the HH or GLI by making substitutions, additions or deletions that provide for functionally equivalent molecules. Preferably, such derivatives are made that have enhanced or increased effect on the proliferation and differentiation of adult brain stem cells.

Due to the degeneracy of nucleotide coding sequences, other DNA sequences which encode substantially the same amino acid sequence as an hh gene may be used in the practice of the present invention including those comprising conservative substitutions thereof. These include but are not limited to modified allelic genes, modified homologous genes from other species, and nucleotide sequences comprising all or portions of hh genes which are altered by the substitution of different codons that encode the same amino acid residue within the sequence, thus producing a silent change. Likewise, the HH derivative of the invention can include, but is not limited to, those containing, as a primary amino acid sequence, all or part of the amino acid sequence of an HH protein including altered sequences in which functionally equivalent amino acid residues are substituted for residues within the sequence resulting in a conservative amino acid substitution. And thus, such substitutions are defined as a conservative substitution.

For example, one or more amino acid residues within the sequence can be substituted by another amino acid of a similar polarity, which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such alterations will not be expected to significantly affect apparent molecular weight as determined by polyacrylamide gel electrophoresis, or isoelectric point.

Particularly preferred conservative substitutions are:

Lys for Arg and vice versa such that a positive charge may be maintained;

Glu for Asp and vice versa such that a negative charge may be maintained;

Ser for Thr such that a free —OH can be maintained; and

Gln for Asn such that a free NH₂ can be maintained.

Amino acid substitutions may also be introduced to substitute an amino acid with a particularly preferable property. For example, a Cys may be introduced at a potential site for disulfide bridges with another Cys. Pro may be introduced because of its particularly planar structure, which induces turns in the protein's structure.

When comparing a particular full-length SHH for example, with human SHH having the amino acid sequence of SEQ ID NO: 2, or human GLI 1, 2 or 3, having the amino sequence of SEQ ID NOs: 16, 18 and 20, respectively, deletions or insertions that could otherwise alter the correspondence between the two amino acid sequences are taken into account. Preferably standard computer analysis is employed for the determination that is comparable, (or identical) to that determined with an Advanced Blast search under the default filter conditions (e.g., using the GCG (Genetics Computer Group, Program Manual for the GCG Package, Version 7, Madison, Wis.) pileup program using the default parameters).

The genes encoding HH or GLI derivatives and analogs of the invention can be produced by various methods known in the art. The manipulations which result in their production can occur at the gene or protein level. For example, an hh or gli gene sequence can be produced from a native hh or gli clone by any of numerous strategies known in the art (Sambrook et al., 1989, supra). The sequence can be cleaved at appropriate sites with restriction endonuclease(s), followed by further enzymatic modification if desired, isolated, and ligated in vitro. In the production of the gene encoding a derivative or analog of a nucleic acid encoding an HH, care should be taken to ensure that the modified gene remains within the same translational reading frame as the hh gene, uninterrupted by translational stop signals, in the gene region where the desired activity is encoded.

Additionally, the HH or GLI-encoding nucleic acid sequence can be produced by in vitro or in vivo mutations, to create and/or destroy translation, initiation, and/or termination sequences, or to create variations in coding regions and/or form new restriction endonuclease sites or destroy preexisting ones, to facilitate further in vitro modification. Preferably such mutations will further enhance the specific properties of the gene products identified to have the capabilities disclosed by the present invention. Any technique for mutagenesis known in the art can be used, including but not limited to, in vitro site-directed mutagenesis (Hutchinson, C., et al., J. Biol. Chem., 253:6551 (1978); Zoller and Smith, DNA, 3:479-488 (1984); Oliphant et al., Gene, 44:177 (1986); Hutchinson et al., Proc. Natl. Acad. Sci. U.S.A., 83:710 (1986)), use of TAB® linkers (Pharmacia), etc. PCR techniques are preferred for site directed mutagenesis (see Higuchi, 1989, “Using PCR to Engineer DNA”, in PCR Technology: Principles and Applications for DNA Amplification, H. Erlich, ed., Stockton Press, Chapter 6, pp. 61-70). A general method for site-specific incorporation of unnatural amino acids into proteins is described in Noren et al., (Science, 244:182-188 (1989)). This method may be used to create analogs with unnatural amino acids.

### Expression of HH and GLI Polypeptides and Active Fragments Thereof

The nucleotide sequence coding for an HH, or GLI or a functionally equivalent derivative including a chimeric protein thereof, can be inserted into an appropriate expression vector, i.e., a vector which contains the necessary elements for the transcription and translation of the inserted protein-coding sequence. Such elements are termed herein a “promoter.” Thus, the nucleic acid encoding an HH or GLI is operationally associated with a promoter in an expression vector of the invention. Both cDNA and genomic sequences can be cloned and expressed under control of such regulatory sequences. An expression vector also preferably includes a replication origin.

The necessary transcriptional and translational signals can be provided on a recombinant expression vector, or they may be supplied by the native gene encoding the corresponding HH or GLI and/or its flanking regions. Any person with skill in the art of molecular biology or protein chemistry, in view of the present disclosure, would readily know how to assay the HH or GLI expressed as described herein, to determine whether such a modified protein can indeed perform the functions of an HH or GLI taught by the present invention. Potential host-vector systems include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used. Expression of an SHH may be controlled by any promoter/enhancer element known in the art, e.g., a Simian Virus 40 (SV40) promoter, a cytomegalus virus promoter (CMV) promoter, or a tissue specific promoter such as the human glial fibrillary acidic protein promoter (GFAP) promoter, as long as these regulatory elements are functional in the host selected for expression. The resulting SHH or GLI protein or fragment thereof can be purified, if desired, by any methodology such as one that is well known in the art.

Production of Cells from Tissue

Cells that can be used in the methods of the present invention can be obtained from tissue specimens, including tumor biopsies by methods known to those skilled in the art.

Once the cells are isolated they can be proliferated and grown in the presence of specific growth medium and any other factor (or molecule) that can maintain cell viability. Thus the cells can be cultured in vitro as described below, in the presence of any other factor (or molecule) that can maintain cell viability until cellular testing for the presence of GLI can be done.

### Antisense Therapy

The relationship between an antisense compound such as an oligonucleotide and its complementary nucleic acid target, to which it hybridizes, is commonly referred to as “antisense”. “Targeting” an oligonucleotide to a chosen nucleic acid target, in the context of this invention, is a multistep process. The process usually begins with identifying a nucleic acid sequence whose function is to be modulated. This may be, as examples, a cellular gene (or mRNA made from the gene) whose expression is associated with a particular disease state. In the present invention, the targets are nucleic acids encoding GLI; in other words, a gene encoding GLI, or mRNA expressed from the gli gene. mRNA which encodes GLI (GLI1, GLI2 or GLI3) is presently the preferred target. The targeting process also includes determination of a site or sites within the nucleic acid sequence for the antisense interaction to occur such that modulation of gene expression will result.

In accordance with this invention, persons of ordinary skill in the art will understand that messenger RNA includes not only the information to encode a protein using the three letter genetic code, but also associated ribonucleotides which form a region known to such persons as the 5′-untranslated region, the 3′-untranslated region, the 5′ cap region and intron/exon junction ribonucleotides. Thus, oligonucleotides may be formulated in accordance with this invention, which are targeted wholly or in part to these associated ribonucleotides as well as to the informational ribonucleotides. The oligonucleotide may therefore be specifically hybridizable with a transcription initiation site region, a translation initiation codon region, a 5′ cap region, an intron/exon junction, coding sequences, a translation termination codon region or sequences in the 5′- or 3′-untranslated region. Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon.” A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene encoding GLI, regardless of the sequence(s) of such codons. It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region,” “AUG region” and “translation initiation codon region” refer to a portion of such an mRNA or gene that encompasses from about 25 to about 50 contiguous nucleotides in either direction (i.e., 5′ or 3′) from a translation initiation codon. This region is a particular target region. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon. This region is a particular target region. The open reading frame (ORF) or “coding region,” which is known in the art to refer to the region between the translation initiation codon and the translation termination codon, is also a region which may be targeted effectively. Other preferred target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The 5′ cap region may also be a preferred target region.

Although some eukaryotic mRNA transcripts are directly translated, many contain one or more regions, known as “introns”, which are excised from a pre-mRNA transcript to yield one or more mature mRNA. The remaining (and therefore translated) regions are known as “exons” and are spliced together to form a continuous mRNA sequence. mRNA splice sites, i.e., exon-exon or intron-exon junctions, may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. Targeting particular exons in alternatively spliced mRNAs may also be preferred. It has also been found that introns can also be effective, and therefore preferred, target regions for antisense compounds targeted, for example, to DNA or pre-mRNA.

Once the target site or sites have been identified, oligonucleotides are chosen which are sufficiently complementary to the target, i.e., hybridize sufficiently well and with sufficient specificity, to give the desired modulation.

“Hybridization”, in the context of this invention, means hydrogen bonding, also known as Watson-Crick base pairing, between complementary bases, usually on opposite nucleic acid strands or two regions of a nucleic acid strand. Guanine and cytosine are examples of complementary bases which are known to form three hydrogen bonds between them. Adenine and thymine are examples of complementary bases which form two hydrogen bonds between them.

“Specifically hybridizable” and “complementary” are terms which are used to indicate a sufficient degree of complementarity such that stable and specific binding occurs between the DNA or RNA target and the oligonucleotide.

It is understood that an oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment or, in the case of in vitro assays, under conditions in which the assays are conducted.

Hybridization of antisense oligonucleotides with mRNA interferes with one or more of the normal functions of mRNA. The functions of mRNA to be interfered with include all vital functions such as, for example, translocation of the RNA to the site of protein translation, translation of protein from the RNA, splicing of the RNA to yield one or more mRNA species, and catalytic activity which may be engaged in by the RNA. Binding of specific protein(s) to the RNA may also be interfered with by antisense oligonucleotide hybridization to the RNA.

The overall effect of interference with mRNA function is modulation of expression of GLI. In the context of this invention “modulation” means either inhibition or stimulation; i.e., either a decrease or increase in expression. This modulation can be measured in ways which are routine in the art, for example by Northern blot assay of mRNA expression, or reverse transcriptase PCR, as taught in the examples of the instant application or by Western blot or ELISA assay of protein expression, or by an immunoprecipitation assay of protein expression. Effects on cell proliferation or tumor cell growth can also be measured, as taught in the examples of the instant application. Inhibition is presently preferred.

The antisense oligonucleotides of this invention can be used in diagnostics, therapeutics, prophylaxis, and as research reagents and in kits. Since the oligonucleotides of this invention hybridize to nucleic acids encoding GLI, sandwich, calorimetric and other assays can easily be constructed to exploit this fact. Provision of means for detecting hybridization of oligonucleotide with the gli gene or mRNA can routinely be accomplished. Such provision may include enzyme conjugation, radiolabelling or any other suitable detection systems. Kits for detecting the presence or absence of GLI may also be prepared.

The present invention is also suitable for diagnosing certain cancers in tissue or other samples from patients suspected of having hyperproliferative condition or cancer such as, but not limited to brain cancer, skin cancer, lung cancer, bladder cancer and prostate cancer. A number of assays may be formulated employing the present invention, which assays will commonly comprise contacting a tissue sample with an oligonucleotide of the invention under conditions selected to permit detection and, usually, quantitation of such inhibition. In the context of this invention, to “contact” tissues or cells with an oligonucleotide or oligonucleotides means to add the oligonucleotide(s), usually in a liquid carrier, to a cell suspension or tissue sample, either in vitro or ex vivo, or to administer the oligonucleotide(s) to cells or tissues within an animal.

The oligonucleotides of this invention may also be used for research purposes. Thus, the specific hybridization exhibited by the oligonucleotides may be used for assays, purifications, cellular product preparations and in other methodologies which may be appreciated by persons of ordinary skill in the art.

In the context of this invention, the term “oligonucleotide” refers to an oligomer or polymer of ribonucleic acid or deoxyribonucleic acid. This term includes oligonucleotides composed of naturally-occurring nucleobases, sugars and covalent intersugar (backbone) linkages as well as oligonucleotides having non-naturally-occurring portions which function similarly. Such modified or substituted oligonucleotides are often preferred over native forms because of desirable properties such as, for example, enhanced cellular uptake, enhanced binding to target and increased stability in the presence of nucleases.

The antisense compounds in accordance with this invention preferably comprise from about 10 to about 50 nucleobases. Particularly preferred are antisense oligonucleotides comprising from about 10 to about 30 nucleobases (i.e. from about 10 to about 30 linked nucleosides). As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2=, 3= or 5=hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3= to 5=phosphodiester linkage.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C or m5c), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808, those disclosed in the Concise Encyclopedia Of Polymer Science And Engineering 1990, pages 858-859, Kroschwitz, J. I., ed. John Wiley & Sons, those disclosed by Englisch et al. (Angewandte Chemie, International Edition 1991, 30, 613-722), and those disclosed by Sanghvi, Y. S., Chapter 15, Antisense Research and Applications 1993, pages 289-302, Crooke, S. T. and Lebleu, B., ed., CRC Press. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2.degree. C. (Sanghvi, Y. S., Crooke, S. T. and Lebleu, B., eds., Antisense Research and Applications 1993, CRC Press, Boca Raton, pages 276-278) and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941.

Another modification of the oligonucleotides of the invention involves chemically linking to the oligonucleotide one or more moieties or conjugates which enhance the activity, cellular distribution or cellular uptake of the oligonucleotide. Such moieties include but are not limited to lipid moieties such as a cholesterol moiety (Letsinger et al., Proc. Natl. Acad. Sci. USA 1989, 86, 6553-6556), cholic acid (Manoharan et al., Bioorg. Med. Chem. Lett. 1994, 4, 1053-1059), a thioether, e.g., hexyl-5-tritylthiol (Manoharan et al., Ann. N.Y. Acad. Sci. 1992, 660, 306-309; Manoharan et al., Bioorg. Med. Chem. Let. 1993, 3, 2765-2770), a thiocholesterol (Oberhauser et al., Nucl. Acids Res. 1992, 20, 533-538), an aliphatic chain, e.g., dodecandiol or undecyl residues (Saison-Behmoaras et al., EMBO J. 1991, 10, 1111-1118; Kabanov et al., FEBS Lett. 1990, 259, 327-330; Svinarchuk et al., Biochimie 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett. 1995, 36, 3651-3654; Shea et al., Nucl. Acids Res. 1990, 18, 3777-3783), a polyamine or a polyethylene glycol chain (Manoharan et al., Nucleosides & Nucleotides 1995, 14, 969-973), or adamantane acetic acid (Manoharan et al., Tetrahedron Lett. 1995, 36, 3651-3654), a palmityl moiety (Mishra et al., Biochim. Biophys. Acta 1995, 1264, 229-237), or an octadecylamine or hexylamino-carbonyl-oxycholesterol moiety (Crooke et al., J. Pharmacol. Exp. Ther. 1996, 277, 923-937).

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

The present invention also includes oligonucleotides which are chimeric oligonucleotides. “Chimeric” oligonucleotides or “chimeras,” in the context of this invention, are oligonucleotides which contain two or more chemically distinct regions, each made up of at least one nucleotide. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of antisense inhibition of gene expression. Cleavage of the RNA target can be routinely detected by gel electrophoresis and, if necessary, associated nucleic acid hybridization techniques known in the art. This RNAse H-mediated cleavage of the RNA target is distinct from the use of ribozymes to cleave nucleic acids.

Examples of chimeric oligonucleotides include but are not limited to “gapmers,” in which three distinct regions are present, normally with a central region flanked by two regions which are chemically equivalent to each other but distinct from the gap. A preferred example of a gapmer is an oligonucleotide in which a central portion (the “gap”) of the oligonucleotide serves as a substrate for RNase H and is preferably composed of 2′-deoxynucleotides, while the flanking portions (the 5′ and 3′ “wings”) are modified to have greater affinity for the target RNA molecule but are unable to support nuclease activity (e.g., fluoro- or 2′-O-methoxyethyl-substituted). Chimeric oligonucleotides are not limited to those with modifications on the sugar, but may also include oligonucleosides or oligonucleotides with modified backbones, e.g., with regions of phosphorothioate (P═S) and phosphodiester (P═O) backbone linkages or with regions of MMI and P═S backbone linkages. Other chimeras include “wingmers,” also known in the art as “hemimers,” that is, oligonucleotides with two distinct regions. In a preferred example of a wingmer, the 5′ portion of the oligonucleotide serves as a substrate for RNase H and is preferably composed of 2′-deoxynucleotides, whereas the 3′ portion is modified in such a fashion so as to have greater affinity for the target RNA molecule but is unable to support nuclease activity (e.g., 2′-fluoro- or 2′-O-methoxyethyl-substituted), or vice-versa. In one embodiment, the oligonucleotides of the present invention contain a 2′-O-methoxyethyl (2′-O—CH.sub.2 CH.sub.2 OCH.sub.3) modification on the sugar moiety of at least one nucleotide. This modification has been shown to increase both affinity of the oligonucleotide for its target and nuclease resistance of the oligonucleotide. According to the invention, one, a plurality, or all of the nucleotide subunits of the oligonucleotides of the invention may bear a 2′-O-methoxyethyl (—O—CH2 CH2 OCH3) modification. Oligonucleotides comprising a plurality of nucleotide subunits having a 2′-O-methoxyethyl modification can have such a modification on any of the nucleotide subunits within the oligonucleotide, and may be chimeric oligonucleotides. Aside from or in addition to 2′-O-methoxyethyl modifications, oligonucleotides containing other modifications which enhance antisense efficacy, potency or target affinity are also contemplated.

The oligonucleotides used in accordance with this invention may be conveniently and routinely made through the well-known technique of solid phase synthesis. Equipment for such synthesis is sold by several vendors including Applied Biosystems. Any other means for such synthesis may also be employed; the actual synthesis of the oligonucleotides is well within the talents of the skilled artisan. It is well known to use similar techniques to prepare oligonucleotides such as the phosphorothioates and 2′-alkoxy or 2′-alkoxyalkoxy derivatives, including 2′-O-methoxyethyl oligonucleotides (Martin, P., Helv. Chim. Acta 1995, 78, 486-504). It is also well known to use similar techniques and commercially available modified amidites and controlled-pore glass (CPG) products such as biotin, fluorescein, acridine or psoralen-modified amidites and/or CPG (available from Glen Research, Sterling, Va.) to synthesize fluorescently labeled, biotinylated or other conjugated oligonucleotides.

The antisense compounds of the present invention include bioequivalent compounds, including pharmaceutically acceptable salts and prodrugs. This is intended to encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to pharmaceutically acceptable salts of the nucleic acids of the invention and prodrugs of such nucleic acids. Pharmaceutically acceptable salts are physiologically and pharmaceutically acceptable salts of the nucleic acids of the invention: i.e., salts that retain the desired biological activity of the parent compound and do not impart undesired toxicological effects thereto (see, for example, Berge et al., “Pharmaceutical Salts,” J. of Pharma Sci. 1977, 66, 1-19).

For oligonucleotides, examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

The oligonucleotides of the invention may additionally or alternatively be prepared to be delivered in a prodrug form. The term prodrug indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention may be prepared as SATE [(S-acetyl-2-thioethyl)phosphate] derivatives according to the methods disclosed in WO 93/24510 to Gosselin et al., published Dec. 9, 1993.

For therapeutic or prophylactic treatment, oligonucleotides are administered in accordance with this invention. Oligonucleotide compounds of the invention may be formulated in a pharmaceutical composition, which may include pharmaceutically acceptable carriers, thickeners, diluents, buffers, preservatives, surface active agents, neutral or cationic lipids, lipid complexes, liposomes, penetration enhancers, carrier compounds and other pharmaceutically acceptable carriers or excipients and the like in addition to the oligonucleotide. Such compositions and formulations are comprehended by the present invention.

Pharmaceutical compositions comprising the oligonucleotides of the present invention (the antisense oligonucleotides and the siRNA molecules described below) may include penetration enhancers in order to enhance the alimentary delivery of the oligonucleotides. Penetration enhancers may be classified as belonging to one of five broad categories, i.e., fatty acids, bile salts, chelating agents, surfactants and non-surfactants (Lee et al., Critical Reviews in Therapeutic Drug Carrier Systems 1991, 8, 91-192; Muranishi, Critical Reviews in Therapeutic Drug Carrier Systems 1990, 7, 1-33). One or more penetration enhancers from one or more of these broad categories may be included. Various fatty acids and their derivatives which act as penetration enhancers include, for example, oleic acid, lauric acid, capric acid, myristic acid, palmitic acid, stearic acid, linoleic acid, linolenic acid, dicaprate, tricaprate, recinleate, monoolein (a.k.a. 1-monooleoyl-rac-glycerol), dilaurin, caprylic acid, arachidonic acid, glyceryl 1-monocaprate, 1-dodecylazacycloheptan-2-one, acylcarnitines, acylcholines, mono- and di-glycerides and physiologically acceptable salts thereof (i.e., oleate, laurate, caprate, myristate, palmitate, stearate, linoleate, etc.) (Lee et al., Critical Reviews in Therapeutic Drug Carrier Systems 1991, page 92; Muranishi, Critical Reviews in Therapeutic Drug Carrier Systems 1990, 7, 1; El-Hariri et al., J. Pharm. Pharmacol. 1992 44, 651-654).

The physiological roles of bile include the facilitation of dispersion and absorption of lipids and fat-soluble vitamins (Brunton, Chapter 38 In: Goodman & Gilman's The Pharmacological Basis of Therapeutics, 9th Ed., Hardman et al., eds., McGraw-Hill, New York, N.Y., 1996, pages 934-935). Various natural bile salts, and their synthetic derivatives, act as penetration enhancers. Thus, the term “bile salt” includes any of the naturally occurring components of bile as well as any of their synthetic derivatives.

Complex formulations comprising one or more penetration enhancers may be used. For example, bile salts may be used in combination with fatty acids to make complex formulations.

Chelating agents include, but are not limited to, disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines) [Lee et al., Critical Reviews in Therapeutic Drug Carrier Systems 1991, page 92; Muranishi, Critical Reviews in Therapeutic Drug Carrier Systems 1990, 7, 1-33; Buur et al., J. Control Rel. 1990, 14, 43-51). Chelating agents have the added advantage of also serving as DNase inhibitors.

Surfactants include, for example, sodium lauryl sulfate, polyoxyethylene-9-lauryl ether and polyoxyethylene-20-cetyl ether (Lee et al., Critical Reviews in Therapeutic Drug Carrier Systems 1991, page 92); and perfluorochemical emulsions, such as FC-43 (Takahashi et al., J. Pharm. Pharmacol. 1988, 40, 252-257).

Non-surfactants include, for example, unsaturated cyclic ureas, 1-alkyl- and 1-alkenylazacyclo-alkanone derivatives (Lee et al., Critical Reviews in Therapeutic Drug Carrier Systems 1991, page 92); and non-steroidal anti-inflammatory agents such as diclofenac sodium, indomethacin and phenylbutazone (Yamashita et al., J. Pharm. Pharmacol. 1987, 39, 621-626).

As used herein, “carrier compound” as used in the context of the oligonucleotides of the present invention, refers to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. In contrast to a carrier compound, a “pharmaceutically acceptable carrier” (excipient) is a pharmaceutically acceptable solvent, suspending agent or any other pharmacologically inert vehicle for delivering one or more nucleic acids to an animal. The pharmaceutically acceptable carrier may be liquid or solid and is selected with the planned manner of administration in mind so as to provide for the desired bulk, consistency, etc., when combined with a nucleic acid and the other components of a given pharmaceutical composition. Typical pharmaceutically acceptable carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrates (e.g., starch, sodium starch glycolate, etc.); or wetting agents (e.g., sodium lauryl sulphate, etc.). Sustained release oral delivery systems and/or enteric coatings for orally administered dosage forms are described in U.S. Pat. Nos. 4,704,295; 4,556,552; 4,309,406; and 4,309,404.

The compositions of the present invention may additionally contain other adjunct components conventionally found in pharmaceutical compositions, at their art-established usage levels. Thus, for example, the compositions may contain additional compatible pharmaceutically-active materials such as, e.g., antipruritics, astringents, local anesthetics or anti-inflammatory agents, or may contain additional materials useful in physically formulating various dosage forms of the composition of present invention, such as dyes, flavoring agents, preservatives, antioxidants, opacifiers, thickening agents and stabilizers. However, such materials, when added, should not unduly interfere with the biological activities of the components of the compositions of the invention.

Regardless of the method by which the oligonucleotides of the invention are introduced into a patient, colloidal dispersion systems may be used as delivery vehicles to enhance the in vivo stability of the oligonucleotides and/or to target the oligonucleotides to a particular organ, tissue or cell type. Colloidal dispersion systems include, but are not limited to, macromolecule complexes, nanocapsules, microspheres, beads and lipid-based systems including oil-in-water emulsions, micelles, mixed micelles, liposomes and lipid:oligonucleotide complexes of uncharacterized structure. A preferred colloidal dispersion system is a plurality of liposomes. Liposomes are microscopic spheres having an aqueous core surrounded by one or more outer layers made up of lipids arranged in a bilayer configuration (see, generally, Chonn et al., Current Op. Biotech. 1995, 6, 698-708).

The pharmaceutical compositions of the present invention may be administered in a number of ways depending upon whether local or systemic treatment is desired and upon the area to be treated. Administration may be topical (including ophthalmic, vaginal, rectal, intranasal, epidermal, and transdermal), oral or parenteral. Parenteral administration includes intravenous drip, subcutaneous, intraperitoneal or intramuscular injection, pulmonary administration, e.g., by inhalation or insufflation, or intracranial, e.g., intrathecal or intraventricular, administration. Oligonucleotides with at least one 2′-O-methoxyethyl modification are believed to be particularly useful for oral administration.

Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets or tablets. Thickeners, flavoring agents, diluents, emulsifiers, dispersing aids or binders may be desirable.

Compositions for parenteral administration may include sterile aqueous solutions which may also contain buffers, diluents and other suitable additives. In some cases it may be more effective to treat a patient with an oligonucleotide of the invention in conjunction with other traditional therapeutic modalities in order to increase the efficacy of a treatment regimen. In the context of the invention, the term “treatment regimen” is meant to encompass therapeutic, palliative and prophylactic modalities. For example, a patient may be treated with conventional chemotherapeutic agents, particularly those used for tumor and cancer treatment. Examples of such chemotherapeutic agents include but are not limited to daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine (CA), 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide, trimetrexate, teniposide, cisplatin and diethylstilbestrol (DES). See, generally, The Merck Manual of Diagnosis and Therapy, 15th Ed. 1987, pp. 1206-1228, Berkow et al., eds., Rahway, N. J. When used with the compounds of the invention, such chemotherapeutic agents may be used individually (e.g., 5-FU and oligonucleotide), sequentially (e.g., 5-FU and oligonucleotide for a period of time followed by MTX and oligonucleotide), or in combination with one or more other such chemotherapeutic agents (e.g., 5-FU, MTX and oligonucleotide, or 5-FU, radiotherapy and oligonucleotide).

The formulation of therapeutic compositions and their subsequent administration is believed to be within the skill of those in the art. Dosing is dependent on severity and responsiveness of the disease state to be treated, with the course of treatment lasting from several days to several months, or until a cure is effected or a diminution of the disease state is achieved.

Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC₅₀ found to be effective in vitro and in in vivo animal models. In general, dosage is from 0.01 μg to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 μg to 100 g per kg of body weight, once or more daily, to once every 20 years.

siRNA Therapy

In general terms, RNA interference (RNAi) is the process whereby the introduction of double stranded RNA into a cell inhibits the expression of a gene corresponding to its own sequence. RNAi is usually described as a post-transcriptional gene-silencing (PTGS) mechanism in which dsRNA triggers degradation of homologous messenger RNA in the cytoplasm. The mediators of RNA interference are 21- and 23-nucleotide small interfering RNAs (siRNA) (Elbashir, S. M. et al., (2001), Genes Dev. 15, 188-200; Elbashir, S. M. et al. (2001), Nature 411: 494-498; Hutvagner, G. et al., (2001), Science 293:834-838). In a second step, siRNAs bind to a ribonuclease complex called RNA-induced silencing complex (RISC) that guides the small dsRNAs to its homologous mRNA target. Consequently, RISC cuts the mRNA approximately in the middle of the region paired with the antisense siRNA, after which the mRNA is further degraded. A ribonuclease III enzyme, dicer, is required for processing of long dsRNA into siRNA duplexes (Bernstein, E. et al. ((2001), Nature 409: 363-366).

### Mechanism of RNAi

The only RNA molecules normally found in the cytoplasm of a cell are molecules of single-stranded mRNA. If the cell finds molecules of double-stranded RNA (dsRNA), it uses a ribonuclease III enzyme, dicer, for processing of long dsRNA into siRNA duplexes (Bernstein, E. et al. ((2001), Nature 409: 363-366) containing ˜22 base pairs (˜2 turns of a double helix). Dicer is a bidentate RNase III, which also contains an ATP-dependent RNA helicase domain and a PAZ domain, presumably important for dsRNA unwinding and mediation of protein-protein interactions, respectively ((Bernstein, E. et al. ((2001), Nature 409: 363-366). Dicer is evolutionarily conserved in worms, flies, plants, fungi and mammals, and has a second cellular function important for the development of these organisms (Grishok, A. (2001), Cell 106:23-34; Knight, S. W. et al. (2001), Science 293:2269-2271; Hutvagner, G. et al., (2001), Science 293:834-838). At present, it is uncertain whether dicer activity in species other than D. melanogaster produces siRNAs of predominantly 21 nt in length. The estimates of siRNA size vary in the literature between 21 and 25 nt (Hamilton, A. J. et al. (1999), Science 286: 950-952; Zamore, P. D. et al. (2000), Cell 101: 25-33; Elbashir, S. M. et al., (2001), Genes Dev. 15, 188-200; Elbashir, S. M. et al. (2001), Nature 411: 494-498; Hammond, S. M. et al. (2000), Nature 404: 293-296; Hutvagner, G. et al., (2001), Science 293:834-838

The two strands of each fragment then separate enough to expose the antisense strand so that it can bind to the complementary sense sequence on a molecule of mRNA. In RNAi, a siRNA-containing endonuclease complex cleaves a single-stranded target RNA in the middle of the region complementary to the 21 nt guide siRNA of the siRNA duplex (Elbashir, S. M. et al., (2001), Genes Dev. 15, 188-200; Elbashir, S. M. et al. (2001), Nature 411: 494-498). This cleavage site is one helical turn displaced from the cleavage site that produced the siRNA from long dsRNA, suggesting dramatic conformational and/or compositional changes after processing of long dsRNA to 21 nt siRNA duplexes. The target RNA cleavage products are rapidly degraded because they either lack the stabilizing cap or poly(A) tail. A protein component of the ˜500 kDa endonuclease or RNA-induced silencing complex (RISC) was recently identified and is a member of the argonaute family of proteins (Hammond, S. M. et al. (2001) Science 293: 1146-1150), however, it is currently unclear whether dicer is required for RISC activity. Thus, the cleavage of the mRNA destroys its ability to be translated into a polypeptide. Because of their action, these fragments of RNA have been named “short (or small) interfering RNA” (siRNA).

Introducing dsRNA corresponding to a particular gene will knock out the cell's own expression of that gene. This can be done in particular tissues at a chosen time. This often provides an advantage over conventional gene “knockouts” where the missing gene is carried in the germline and thus whose absence may kill the embryo before it can be studied.

Although it has been suggested that the one disadvantage of simply introducing dsRNA fragments into a cell is that gene expression is only temporarily reduced, it has recently been shown that the system can be manipulated using a DNA vector such that the siRNA molecule can be continuously synthesized for prolonged periods of time in order to continue in suppression of the desired gene (Brummelkamp et. al. 19 Apr. 2002, Science). After two months, the cells still failed to manufacture the protein whose gene had been turned off by RNAi. Effective siRNA molecules may be designed using the following guidelines:

In general, siRNA oligonucleotides should be about 21 nucleotides in length with 2 nucleotide overhangs, usually 3′ TT.

Sequences located in the 5′ or 3′ UTR of the mRNA target and nearby the start codon should be avoided, as they may be richer in regulatory protein binding sites.

Search for a sequence AA(N19)TT or AA(N21) with approximately 50% G/C content.

Compare the selected siRNA nucleotide sequence against databases to ensure that only one gene will be targeted.

Target recognition is a highly sequence specific process, mediated by the siRNA complementary to the target. One or two base pair mismatches between the siRNA and the target gene will greatly reduce the silencing effect. It might be necessary to test several sequences since positional effects of siRNAs have been reported.

The 3′-most nucleotide of the guide siRNA does not contribute to the specificity of target recognition, while the penultimate nucleotide of the 3′ overhang affects target RNA cleavage and a mismatch reduces RNAi 2- to 4-fold. The 5′ end of the guide siRNA also appears more permissive for mismatched target RNA recognition when compared with the 3′ end. Nucleotides in the center of the siRNA, located opposite to the target RNA cleavage site, are important specificity determinants and even single nucleotide changes reduce RNAi to undetectable levels. This suggests that siRNA duplexes may be able to discriminate mutant or polymorphic alleles in gene targeting experiments, which may become an important feature for future therapeutic developments.

Double-stranded RNA has been shown to attenuate specific gene expression in C. elegans, Drosophila and Trypanosoma brucei (M. Montgomery, et al., Proc. Natl. Acad. Sci. U.S.A. 95, 15502-15507 (1998); J. Kennerdell et al., Cell 95, 1017-1026 (1998); H. Ngo et al., Proc. Natl. Acad. Sci. U.S.A. 95, 14687-14692 (1998)). The types of genes attenuated in these invertebrates include some encoding transcription factors and others that encode growth factor receptors. There is also evidence that double-stranded RNA may effectively silence gene expression in plants (M. Wassenegger et al., Plant. Mol. Biol. 37, 349-362 (1998); P. Watergiyse et al., Proc. Natl. Acad. Sci. U.S.A. 95, 13959-13964 (1998)).

A definitive mechanism through which double-stranded RNA effects gene silencing remains has not been identified (M. Montgomery et al., Trends Genet. 14, 255-258 (1998)). Recently, Montgomery et al. reported that double-stranded RNA induces specific RNA degradation in nematodes (Proc. Natl. Acad. Sci. U.S.A. 95, 15502-15507 (1998)). This conclusion was based upon the fact that DNA sequences in the targeted regions of the gene were not altered and that 100% of the F2 generation reverted to the wild type phenotype. In addition, C. elegans has a unique genetic organization. Genes in this animal are organized in operons in which a single promoter controls expression of a number of genes. They showed that the double-stranded RNA affects only expression of the targeted gene. In contrast, however, others have observed heritable effects of double-stranded RNA on the expression of a number of genes in C. elegans, suggesting that more than one mechanism may be involved in double-stranded RNA-mediated inhibition of gene activity (H. Tahara, Science 28, 431-432 (1998)).

The present invention provides a method for attenuating gene expression in a cell using gene-targeted double-stranded RNA (dsRNA). The dsRNA contains a nucleotide sequence that is essentially identical to the nucleotide sequence of at least a portion of the target gene, in the matter of the present invention, the shh or gli genes. The cell into which the dsRNA is introduced is preferably a tumor cell containing at least one gli gene to which the dsRNA is targeted. Gene expression can be attenuated in a whole organism, an organ or tissue of an organism, including a tissue explant, or in cell culture. Preferably, the cell is a mammalian cell, but the invention is not limited to mammals. Double-stranded RNA is introduced directly into the cell or, alternatively, into the extracellular environment from which it is taken up by the cell. Inhibition is specific for the targeted gene. Depending on the particular target gene and the dose of dsRNA delivered, the method may partially or completely inhibit expression of the gene in the cell. The expression of two or more genes can be attenuated concurrently by introducing two or more double stranded RNAs into the cell in amounts sufficient to attenuate expression of their respective target genes. Double stranded RNAs that are administered “concurrently” are administered, together or separately, so as to be effective at generally the same time.

In yet another aspect, the invention provides a method for attenuating the expression of a gli gene in a cell that includes annealing two complementary single stranded RNAs in the presence of potassium chloride to yield double stranded RNA; contacting the double stranded RNA with RNAse to purify the double stranded RNA by removing single stranded RNA; and introducing the purified double stranded RNA into the cell in an amount sufficient to attenuate expression of the target gene, e.g. at least one of the gli genes.

The invention further provides a method for treating or preventing a hyperproliferative condition or a cancerous condition in a mammal. Double stranded RNA is administered to the mammal in an amount sufficient to attenuate expression of the gli gene, the expression of which is associated with the cancerous condition. Concurrent inhibition of multiple genes is advantageous to treat diseases associated with multiple genes, or to treat two or more diseases or infections concurrently.

The present invention provides a method for gene silencing in organisms and cells, especially mammals, using gene-specific double-stranded RNA. The ability to use double-stranded RNA to specifically block expression of particular genes in a multicellular setting both in vivo and in vitro has broad implications for the study of numerous diseases, in the matter of the present invention, cancerous conditions.

The method of the present invention allows for attenuation of gene expression in a cell. “Attentuation of gene expression” can take the form of partial or complete inhibition of gene function Mechanistically, gene function can be partially or completely inhibited by blocking transcription from the gene to mRNA, or by blocking translation of the mRNA to yield the protein encoded by the gene, although it should be understood that the invention is not limited to any particular mechanism of attenuation of gene expression. Inhibition of gene function is evidenced by a reduction or elimination, in the cell, of the activity associated with the protein encoded by the gene. Whether and to what extent gene function is inhibited can be determined using methods known in the art For example, in many cases inhibition of gene function leads to a change in phenotype which is revealed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracyclin.

Attenuation of gene expression can be quantified, and the amount of attenuation of gene expression in a treated cell compared to a cell not treated according to the present invention can be determined. Lower doses dsRNA may result in inhibition in a smaller fraction of cells, or in partial inhibition in cells. In addition, attenuation of gene expression can be time-dependent; the longer the period of time since the administration of the dsRNA, the less gene expression may be attenuated. Attenuation of gene expression can occur at the level of transcription (i.e., accumulation of mRNA of the targeted gene), or translation (i.e., production of the protein encoded by the targeted gene). For example, mRNA from the targeted gene can be detected using a hybridization probe having a nucleotide sequence outside the region selected for the inhibitory double-stranded RNA, and translated polypeptide encoded by the target gene can be detected via Western blotting using an antibody raised against the polypeptide. It should be noted that the method of the invention is not limited to any particular mechanism for reducing or eliminating cellular protein activity; indeed, as noted above, it is not yet fully understood how the introduction of dsRNA into a cell causes attenuation of expression of the targeted gene, nor is it known whether single or multiple mechanisms are at work.

The attenuation of gene expression achieved by the method of the invention is specific for the gli genes, that is, gli1, gli2 or gli3. In other words, the dsRNA inhibits at least one of the target genes without manifest effects on other genes of the cell.

### Double-Stranded RNA

The dsRNA is formed from one or more strands of polymerized ribonucleotide. When formed from only one strand, it takes the form of a self-complementary hairpin-type molecule that doubles back on itself to form a duplex. When formed from two strands, the two strands are complementary RNA strands. The dsRNA can include modifications to either the phosphate-sugar backbone or the nucleoside. For example, the phosphodiester linkages of natural RNA may be modified to include at least one of a nitrogen or sulfur heteroatom. Likewise, bases may be modified to block the activity of adenosine deaminase.

The nucleotide sequence of the dsRNA is defined by the nucleotide sequence of its targeted gene, ie. gli1, gli2 or gli3 (Seq ID NOs: 15, 17, and 19, respectively). The dsRNA contains a nucleotide sequence that is essentially identical to at least a portion of the target gene; preferably the dsRNA contains a nucleotide sequence that is completely identical to at least a portion of the target gene. It should be understood that in comparing an RNA sequence to a DNA sequence, an “identical” RNA sequence will contain ribonucleotides where the DNA sequence contains deoxyribonucleotides, and further that the RNA sequence will contain a uracil at positions where the DNA sequence contains thymidine. More preferably, the dsRNA that is completely identical to at least a portion of the target gene does not contain any additional nucleotides.

A dsRNA that is “essentially identical” to a least a portion of the target gene, e.g. gli, is a dsRNA wherein one of the two complementary stands (or, in the case of a self-complementary RNA, one of the two self-complementary portions) is either identical to the sequence of that portion of the target gene or contains one or more insertions, deletions or single point mutations relative to the nucleotide sequence of that portion of the target gene. The invention thus has the advantage of being able to tolerate sequence variations that might be expected due to genetic mutation, strain polymorphism, or evolutionary divergence. Alternatively, a dsRNA that is “essentially identical” to at least a portion of the target gene can be functionally as a dsRNA wherein one of the two complementary strands (or, in the case of a self-complementary RNA, one of the two self-complementary portions) is capable of hybridizing with a portion of the target gene transcript (e.g., under conditions including 400 mM NaCl, 40 mM PIPES pH 6.4, 1 mM EDTA, 50° C. or 70° C. hybridization for 12-16 hours; followed by washing).

The dsRNA nucleotide sequence that is essentially or completely identical to at least a portion of the target gene has a length of preferably at least about 5-10 bases, more preferably 10-25 bases, more preferably at least about 50 bases, and most preferably at least about 100 bases. The dsRNA nucleotide sequence has a length of preferably less than about 400 bases, more preferably less than about 300 base, more preferably less than about 200 bases and most preferably less than about 100 bases. It will be understood that the length of the dsRNA, the degree of homology necessary to affect gene expression, and the most effective dosages can be optimized for each particular application using routine methods.

Synthesis of dsRNA

Single strands of RNA are synthesized in vitro. Preferably, single stranded RNA is enzymatically synthesized from the PCR products of a DNA template, preferably a cloned a cDNA template. Provided the sequence of the target gene is known, e.g. gli, a cloned cDNA template can be readily made from target cell RNA using reverse-transcriptase polymerase chain reaction (RT-PCR) to generate a cDNA fragment, following by cloning the cDNA fragment into a suitable vector. Preferably, the vector is designed to allow the generation of complementary forward and reverse PCR products. The vector pGEM-T (Promega, Madison Wis.) is well-suited for use in the method because it contains a cloning site positioned between oppositely oriented promoters (i.e., T7 and SP6 promoters; T3 promoter could also be used). After purification of the PCR products, complementary single stranded RNAs are synthesized, in separate reactions, from the DNA templates via RT-PCR using two different RNA polymerases (e.g., in the case of pGEM-T, T7 polymerase and SP6 polymerase). RNAse-free DNAse is added to remove the DNA template, then the single-stranded RNA is purified. Single strands of RNA can also be produced enzymatically or by partial/total organic synthesis. The use of in vitro enzymatic or organic synthesis allows the introduction of any desired modified ribonucleotide. The RNA strands may or may not be polyadenylated; and the RNA strands may or may not be capable of being translated into a polypeptide by a cell's translational apparatus. Preferably, purification of RNA is performed without the use of phenol or chloroform.

Double stranded RNA is formed in vitro by mixing complementary single stranded RNAs, preferably in a molar ratio of at least about 3:7, more preferably in a molar ratio of about 4:6, and most preferably in essentially equal molar amounts (i.e., a molar ratio of about 5:5). Preferably, the single stranded RNAs are denatured prior to annealing, and the buffer in which the annealing reaction takes place contains a salt, preferably potassium chloride. Prior to administration, the mixture containing the annealed (i.e., double stranded) RNA is preferably treated with an enzyme that is specific for single stranded RNA (for example, RNAse A or RNAse T) to confirm annealing and to degrade any remaining single stranded RNAs. Addition of the RNAse also serves to excise any overhanging ends on the dsRNA duplexes.

Delivery of dsRNA to a Cell

Double stranded RNA can be introduced into the cell in a number of different ways. For example, the dsRNA is conveniently administered by microinjection; other methods of introducing nucleic acids into a cell include bombardment by particles covered by the dsRNA, soaking the cell or organism in a solution of the dsRNA, electroporation of cell membranes in the presence of the dsRNA, liposome-mediated delivery of dsRNA and transfection mediated by chemicals such as calcium phosphate, viral infection, transformation, and the like. The dsRNA may be introduced along with components that enhance RNA uptake by the cell, stabilize the annealed strands, or otherwise increase inhibition of the target gene. In the case of a cell culture or tissue explant, the cells are conveniently incubated in a solution containing the dsRNA or lipid-mediated transfection; in the case of a whole animal or plant, the dsRNA is conveniently introduced by injection or perfusion into a cavity or interstitial space of an organism, or systemically via oral, topical, parenteral (including subcutaneous, intramuscular and intravenous administration), vaginal, rectal, intranasal, ophthalmic, or intraperitoneal administration. In addition, the dsRNA can be administered via and implantable extended release device. Methods for oral introduction include direct mixing of RNA with food of the organism, as well as engineered approaches in which a species that is used as food is engineered to express an RNA, then fed to the organism to be affected. The dsRNA may be sprayed onto a plant or a plant may be genetically engineered to express the RNA in an amount sufficient to kill some or all of a pathogen known to infect the plant.

Alternatively, dsRNA can be supplied to a cell indirectly by introducing one or more vectors that encode both single strands of a dsRNA (or, in the case of a self-complementary RNA, the single self-complementary strand) into the cell. Preferably, the vector contains 5′ and 3′ regulatory elements that facilitate transcription of the coding sequence. Single stranded RNA is transcribed inside the cell, and, presumably, double stranded RNA forms and attenuates expression of the target gene. Methods for supplying a cell with dsRNA by introducing a vector from which it can be transcribed are set forth in WO 99/32619 (Fire et al., published Jul. 1, 1999). A transgenic animal that expresses RNA from such a recombinant construct may be produced by introducing the construct into a zygote, an embryonic stem cell, or another multipotent cell derived from the appropriate organism. A viral construct packaged into a viral particle would accomplish both efficient introduction of an expression construct into the cell and transcription of RNA encoded by the expression construct.

The dsRNA is typically administered in an amount that allows delivery of at least one copy per cell. The amount of dsRNA administered to a cell, tissue, or organism depends on the nature of the cell, tissue, or organism, the nature of the target gene, and the nature of the dsRNA, and can readily be optimized to obtain the desired level of gene inhibition. To attenuate gene expression in a single cell embryo, for example, at least about 0.8×10⁶ molecules of dsRNA are injected; more preferably, at least about 20×10⁶ molecules of dsRNA are injected; most preferably, at least about 50×10⁶ molecules of dsRNA are injected. The amount of dsRNA injected into a single cell embryo is, however, preferably at most about 1000×10⁶ molecules; more preferably, it is at most about 500×10⁶ molecules, most preferably, at most about 100×10⁶ molecules. In the case of administration of dsRNA to a cell culture or to cells in tissue, by methods other than injection, for example by soaking, electroporation, or lipid-mediated transfection, the cells are preferably exposed to similar levels of dsRNA in the medium. For example, 8-10 μL of cell culture or tissue can be contacted with about 20×10⁶ to about 2000×10⁶ molecules of dsRNA, more preferably about 100×10⁶ to about 500×10⁶ molecules of dsRNA, for effective attenuation of gene expression.

Once the minimum effective length of the dsRNA has been determined, it is routine to determine the effects of dsRNA agents that are produced using synthesized oligoribonucleotides. The administration of the dsRNA can be by microinjection or by other means used to deliver nucleic acids to cells and tissues, including culturing the tissue in medium containing the dsRNA.

The siRNA molecules of the present invention may be used to introduce dsRNA into a cell for the treatment or prevention of disease. To treat or prevent a disease or other pathology, a target gene is selected which is required for initiation or maintenance of the disease/pathology. The dsRNA can be introduced into the organism using in vitro, ex vivo or by in vivo methods. In an in vitro method, the dsRNA is introduced into a cell, which may or may not be a cell of the organism, and the dsRNA-containing cell is then introduced into the organism. In an ex vivo method, cells of the organism are explanted, the dsRNA is introduced into the explanted cells, and the dsRNA-containing cells are implanted back into the host. In an in vivo method, dsRNA is administered directly to the organism. As noted above, the dsRNA can also be delivered to a cell using one or more vectors that encode the complementary RNAs (or self-complementary RNA), which are then transcribed inside the cell and annealed to yield the desired dsRNA.

In medical applications, the dsRNA may be introduced into a cancerous cell or tumor, and thereby inhibit expression of a gene required for maintenance of the carcinogenic/tumorigenic phenotype.

### Assessment of Prostate Tumor Cell Invasiveness

In order to assess the likelihood or potential for a prostate tumor to metastasize in an individual diagnosed with prostate cancer, the Gleason score is used by most physicians to aid in this assessment. The “Gleason score or Gleason grade” refers to a means for grading prostate cancer and relates to the degree of aggressiveness of the tumor. This grade imparts a significant correlation to the potential prognosis and is an important factor in recommending a particular therapy for that patient. The Gleason system is based on the architectural pattern of the glands of the prostate tumor. A tumor whose structure is nearly normal (well differentiated) will probably have a biological behavior relatively close to normal—not very aggressively malignant. The Gleason grade and score are key pieces of information for making treatment decisions.

Gleason Grades 1 and 2: These two grades closely resemble normal prostate. They are the least important grades because they seldom occur in the general population and because they confer a prognostic benefit which is only slightly better than grade 3. Both of these grades are composed by very pale glands which grow closely together. In grade 1 they form a compact mass; in grade 2 they are more loosely aggregated, and some glands wander (invade) into the surrounding muscle (stroma).

Gleason Grade 3: This is the most common grade by far and is also considered well differentiated (like grades 1 and 2). This is because all three grades have a normal “gland unit” like that of a normal prostate; that is, every cell is part of a circular row which forms the lining of a central space (the lumen). The lumen contains prostatic secretion like normal prostate, and each gland unit is surrounded by prostate muscle which keeps the gland units apart. In contrast to grade 2, wandering of glands (invading) into the stroma (muscle) is very prominent and is the main defining feature. The cells are dark rather than pale and the glands often have more variable shapes

Gleason Grade 4: This is probably the most important grade because it is fairly common and because of the fact that if a lot of it is present, patient prognosis is usually (but not always) worsened by a considerable degree. Here also there is a big jump in loss of architecture. For the first time, we see disruption and loss of the normal gland unit. In fact, grade 4 is identified almost entirely by loss of the ability to form individual, separate gland units, each with its separate lumen.

Gleason Grade 5: Gleason grade 5 is an important grade because it usually predicts another significant step towards poor prognosis. Its overall importance for the general population is reduced by the fact that it is less common than grade 4, and it is seldom seen in men whose prostate cancer is diagnosed early in its development. This grade too shows a variety of patterns, all of which demonstrate no evidence of any attempt to form gland units. Although never an absolute the results with any form of conventional therapy is poor with this category.

The “Combined Gleason Score or Gleason Sum” can be explained as follows. When a pathologist looks at prostate cancer specimens under the microscope and gives them a Gleason Grade, he or she in fact will always try to identify two architectural patterns and assign a Gleason Grade to each one. There may be a primary pattern and then a secondary pattern which the pathologist will seek to describe for each specimen; alternatively, there may often be only a single pure grade.

In developing his system, Dr. Gleason discovered that by giving a combination of the grades of the two most common patterns he could see in any particular patient's specimens, he was better able to predict the likelihood that that particular patient would do well or badly. Therefore, even though it may seem confusing, the Gleason score which a physician usually gives to a patient is actually a combination or sum of two numbers. These combined Gleason sums or scores may be determined as follows:

The lowest possible Gleason score is 2 (1+1), where both the primary and secondary patterns have a Gleason Grade of 1 and therefore when added together their combined sum is 2. Very typical Gleason scores might be 5 (2+3), where the primary pattern has a Gleason grade of 2 and the secondary pattern has a grade of 3, or 6 (3+3), a pure pattern. Another typical Gleason score might be 7 (4+3), where the primary pattern has a Gleason grade of 4 and the secondary pattern has a grade of 3.

Finally, the highest possible Gleason score is 10 (5+5), when the primary and secondary patterns both have the most disordered Gleason grades of 5.

The grade of a prostate cancer specimen is very valuable to doctors in helping them to understand how a particular case of prostate cancer can be treated. In general, the time for which a patient is likely to survive following a diagnosis of prostate cancer is related to the Gleason score. The lower the Gleason score, the better the patient is likely to do. However, remember that prostate cancer is a very complicated disease. People with low Gleason scores have been known to fare poorly and men with high Gleason scores have been known to do well. General principles do not always apply to individual patients.

### Pharmaceutical Compositions

In yet another aspect of the present invention, provided are pharmaceutical compositions of the inhibitors or antagonists of GLI, including the nucleic acids described above, but also the small molecule inhibitors and antibodies that block SHH/GLI signaling. Such pharmaceutical compositions may be for administration for nasal or other forms of administration. In general, comprehended by the invention are pharmaceutical compositions comprising effective amounts of a low molecular weight component or components, or derivative products, of the invention together with pharmaceutically acceptable diluents, preservatives, solubilizers, emulsifiers, adjuvants and/or carriers. Such compositions include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Hylauronic acid may also be used. Such compositions may influence the physical state, stability, rate of in vivo release, and rate of in vivo clearance of the present proteins and derivatives. See, e.g., Remington's Pharmaceutical Sciences, 18th Ed. (1990), Mack Publishing Co., Easton, Pa. 18042 pages 1435-1712 which are herein incorporated by reference. The compositions may be prepared in liquid form, or may be in dried powder, such as lyophilized form.

### Nasal Delivery

Nasal delivery of an HH protein or derivative thereof is also contemplated. Nasal delivery allows the passage of the protein to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

### Liquid Aerosol Formulations

The present invention provides aerosol formulations and dosage forms. In general such dosage forms contain a pharmaceutical composition of the present invention in a pharmaceutically acceptable diluent. Pharmaceutically acceptable diluents include but are not limited to sterile water, saline, buffered saline, dextrose solution, and the like.

The formulation may include a carrier. The carrier is a macromolecule which is soluble in the circulatory system and which is physiologically acceptable where physiological acceptance means that those of skill in the art would accept injection of said carrier into a patient as part of a therapeutic regime. The carrier preferably is relatively stable in the circulatory system with an acceptable plasma half life for clearance. Such macromolecules include but are not limited to Soya lecithin, oleic acid and sorbitan trioleate, with sorbitan trioleate preferred.

The formulations of the present embodiment may also include other agents useful for pH maintenance, solution stabilization, or for the regulation of osmotic pressure.

### Aerosol Dry Powder Formulations

It is also contemplated that the present aerosol formulation can be prepared as a dry powder formulation comprising a finely divided powder form of pharmaceutical composition of the present invention and a dispersant. Formulations for dispensing from a powder inhaler device will comprise a finely divided dry powder containing pharmaceutical composition of the present invention (or derivative) and may also include a bulking agent, such as lactose, sorbitol, sucrose, or mannitol in amounts which facilitate dispersal of the powder from the device, e.g., 50 to 90% by weight of the formulation. The pharmaceutical composition of the present invention (or derivative) should most advantageously be prepared in particulate form with an average particle size of less than 10 mm (or microns), most preferably 0.5 to 5 mm, for most effective delivery to the distal lung.

In a further aspect, recombinant cells that have been transformed with an hh gene, e.g., sonic hedgehog gene and that express high levels of the polypeptide can be transplanted in a subject in need of the HH protein. Preferably autologous cells transformed with HH protein are transplanted to avoid rejection; alternatively, technology is available to shield non-autologous cells that produce soluble factors within a polymer matrix that prevents immune recognition and rejection.

### Methods of Treatment, Methods of Preparing a Medicament

In yet another aspect of the present invention, methods of treatment and manufacture of a medicament are provided. Conditions alleviated or modulated by the administration of the present derivatives are those indicated above.

Dosages. For all of the above molecules, as further studies are conducted, information will emerge regarding appropriate dosage levels for treatment of various conditions in various patients, and the ordinary skilled worker, considering the therapeutic context, age and general health of the recipient, will be able to ascertain proper dosing.

A subject in whom administration of HH is an effective therapeutic regiment is preferably a human, but can be any animal. Thus, as can be readily appreciated by one of ordinary skill in the art, the methods and pharmaceutical compositions of the present invention are particularly suited to administration to any animal, particularly a mammal, and including, but by no means limited to, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

### Antibodies to GLI for Therapeutic or Diagnostic Use

According to the present invention, GLI, as produced by a recombinant source, or through chemical synthesis, or isolated from natural sources; and derivatives or analogs thereof, including fusion proteins, may be used as an immunogen to generate antibodies that recognize the GLI, as exemplified below. Such antibodies include but are not limited to polyclonal, monoclonal, chimeric including humanized chimeric, single chain, Fab fragments, and a Fab expression library. The anti-GLI antibodies, for example, of the invention may be cross reactive, that is, they may recognize a GLI derived from a different source. Polyclonal antibodies have greater likelihood of cross reactivity. Alternatively, an antibody of the invention may be specific for a single form of GLI, such as the human GLI proteins GLI! 1, 2 or 3, having the amino acid sequences of SEQ ID NOs: 16, 18 or 20, respectively, or a fragment of a human GLI protein.

Various procedures known in the art may be used for the production of polyclonal antibodies to GLI or derivatives or analogs thereof. For the production of an antibody, various host animals can be immunized by injection with GLI, or a derivative (e.g., or fusion protein) thereof, including but not limited to rabbits, mice, rats, sheep, goats, etc. In one embodiment, GLI or a fragment thereof can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward GLI, or analog, or derivative thereof, any technique that provides for the production of antibody molecules by continuous cell lines in culture may be used. These include but are not limited to the hybridoma technique originally developed by Kohler and Milstein [Nature, 256:495-497 (1975)], as well as the trioma technique, the human B-cell hybridoma technique [Kozbor et al., Immunology Today, 4:72 (1983); Cote et al., Proc. Natl. Acad. Sci. U.S.A., 80:2026-2030 (1983)], and the EBV-hybridoma technique to produce human monoclonal antibodies [Cole et al., in Monoclonal Antibodies and Cancer Therapy, Alan R. Liss, Inc., pp. 77-96 (1985)]. In an additional embodiment of the invention, monoclonal antibodies can be produced in germ-free animals utilizing recent technology [PCT/US90/02545]. In fact, according to the invention, techniques developed for the production of “chimeric antibodies” [Morrison et al., J. Bacteriol., 159:870 (1984); Neuberger et al., Nature, 312:604-608 (1984); Takeda et al., Nature, 314:452-454 (1985)] by splicing the genes from a mouse antibody molecule specific for GLI together with genes from a human antibody molecule of appropriate biological activity can be used; such antibodies are within the scope of this invention. Such human or humanized chimeric antibodies are preferred for use in therapy of human diseases or disorders (described infra), since the human or humanized antibodies are much less likely than xenogenic antibodies to induce an immune response, in particular an allergic response, themselves.

According to the invention, techniques described for the production of single chain antibodies [U.S. Pat. Nos. 5,476,786 and 5,132,405 to Huston; U.S. Pat. No. 4,946,778] can be adapted to produce e.g., GLI-specific single chain antibodies. An additional embodiment of the invention utilizes the techniques described for the construction of Fab expression libraries [Huse et al., Science, 246:1275-1281 (1989)] to allow rapid and easy identification of monoclonal Fab fragments with the desired specificity for a GLI, or its derivatives, or analogs.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent.

In the production of antibodies, screening for the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), Western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. For example, to select antibodies which recognize a specific epitope of GLI, one may assay generated hybridomas for a product which binds to the GLI fragment containing such epitope and choose those which do not cross-react with GLI. For selection of an antibody specific to GLI from a particular source, one can select on the basis of positive binding with GLI expressed by or isolated from that specific source.

The foregoing antibodies can be used in methods known in the art relating to the localization and activity of the GLI, e.g., for Western blotting, imaging GLI in situ, measuring levels thereof in appropriate physiological samples, etc. using any of the detection techniques mentioned herein or known in the art. The standard techniques known in the art for immunoassays are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. (1984) J. Clin. Chem. Clin. Biochem. 22:895-904.

In a specific embodiment, antibodies that agonize or antagonize the activity of GLI can be generated. Such antibodies can be tested using the assays described infra for identifying ligands.

One aspect of the invention provides a method of using an antibody against GLI to diagnose an invasive or aggressive form of prostate cancer in a subject. As GLI levels correlate with the invasiveness of a prostate tumor as determined by the Gleason score, it provides a general biomarker for highly invasive prostate tumors, and may be predictive of the invasiveness of other tumors that utilize the SHH-GLI signaling pathway. Thus, the antibody compositions and methods provided herein are particularly deemed useful for the diagnosis of invasive tumors including solid tumors such as prostate tumors, as well as other tumors that utilize the SHH-GLI signaling pathway. More particularly, cancers that may be treated by the compositions and methods of the invention include, but are not limited to, Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma [squamous cell, undifferentiated small cell, undifferentiated large cell, adenocarcinoma], alveolar [bronchiolar] carcinoma, bronchial adenoma, sarcoma, lymphoma, chondromatous hamartoma, mesothelioma; Gastrointestinal: esophagus [squamous cell carcinoma, adenocarcinoma, leiomyosarcoma, lymphoma], stomach [carcinoma, lymphoma, leiomyosarcoma], pancreas [ductal adenocarcinoma, insulinoma, glucagonoma, gastrinoma, carcinoid tumors, VIPoma], small bowel [adenocarcinoma, lymphoma, carcinoid tumors, Kaposi's sarcoma, leiomyoma, hemangioma, lipoma, neurofibroma, fibroma], large bowel [adenocarcinoma, tubular adenoma, villous adenoma, hamartoma, leiomyoma]; Genitourinary tract: kidney [adenocarcinoma, Wilms tumor (nephroblastoma), lymphoma, leukemia], bladder and urethra [squamous cell carcinoma, transitional cell carcinoma, adenocarcinoma], prostate [adenocarcinoma, sarcoma], testis [seminoma, teratoma, embryonal carcinoma, teratocarcinoma, choriocarcinoma, sarcoma, Leydig cell tumor, fibroma, fibroadenoma, adenomatoid tumors, lipoma]; Liver: hepatocellular carcinoma, cholangiocarcinoma, hepatoblastoma, angiosarcoma, hepatocellular adenoma, hemangioma; Bone: osteogenic sarcoma [osteosarcoma], fibrosarcoma, malignant fibrous histiocytoma, chondrosarcoma, Ewing's sarcoma, malignant lymphoma [reticulum cell sarcoma], multiple myeloma, malignant giant cell tumor, chordoma, osteochondroma [osteocartilaginous exostoses], benign chondroma, chondroblastoma, chondromyxoid fibroma, osteoid osteoma and giant cell tumors; Nervous system: skull [osteoma, hemangioma, granuloma, xanthoma, Paget's disease of bone], meninges [meningioma, meningiosarcoma, gliomatosis], brain [astrocytoma, medulloblastoma, glioma, ependymoma, germinoma (pinealoma), glioblastoma multiforme, oligodendroglioma, schwannoma, retinoblastoma, congenital tumors], spinal cord [neurofibroma, meningioma, glioma, sarcoma]; Gynecological: uterus [endometrial carcinoma], cervix [cervical carcinoma, pre-invasive cervical dysplasia], ovaries [ovarian carcinoma (serous cystadenocarcinoma, mucinous cystadenocarcinoma, endometrioid carcinoma, clear cell adenocarcinoma, unclassified carcinoma), granulosa-theca cell tumors, Sertoli-Leydig cell tumors, dysgerminoma, malignant teratoma and other germ cell tumors], vulva [squamous cell carcinoma, intraepithelial carcinoma, adenocarcinoma, fibrosarcoma, melanoma], vagina [clear cell carcinoma, squamous cell carcinoma, sarcoma botryoides (embryonal rhabdomyosarcoma), fallopian tubes [carcinoma]; Hematologic: blood [myeloid leukemia (acute and chronic), acute lymphoblastic leukemia, chronic lymphocytic leukemia, myeloproliferative diseases, multiple myeloma, myelodysplastic syndrome], Hodgkin's disease, non-Hodgkin's lymphoma (malignant lymphoma); Skin: malignant melanoma, basal cell carcinoma, squamous cell carcinoma, Kaposi's sarcoma, nevi, dysplastic nevi, lipoma, angioma, dermatofibroma, keloids, psoriasis; and Adrenal glands: neuroblastoma.

The diagnostic method of the invention provides contacting a biological sample such as a biopsy sample, tissue, cell or fluid (e.g., whole blood, plasma, serum, or urine) isolated from a subject with an antibody which binds GLI. The antibody is allowed to bind to the antigen to form an antibody-antigen complex. The conditions and time required to form the antibody-antigen complex may vary and are dependent on the biological sample being tested and the method of detection being used. Once non-specific interactions are removed by, for example, washing the sample, the antibody-antigen complex is detected using any one of the immunoassays described above as well a number of well-known immunoassays used to detect and/or quantitate antigens [see, for example, Harlow and Lane, Antibodies: A Laboratory Manual, Cold Spring Harbor Laboratory, New York (1988) 555-612]. Such well-known immunoassays include antibody capture assays, antigen capture assays, and two-antibody sandwich assays. In an antibody capture assay, the antigen is attached to solid support, and labeled antibody is allowed to bind. After washing, the assay is quantitated by measuring the amount of antibody retained on the solid support. In an antigen capture assay, the antibody is attached to a solid support, and labeled antigen is allowed to bind. The unbound proteins are removed by washing, and the assay is quantitated by measuring the amount of antigen that is bound. In a two-antibody sandwich assay, one antibody is bound to a solid support, and the antigen is allowed to bind to this first antibody. The assay is quantitated by measuring the amount of a labeled second antibody that binds to the antigen.

These immunoassays typically rely on labeled antigens, antibodies, or secondary reagents for detection. These proteins may be labeled with radioactive compounds, enzymes, biotin, or fluorochromes. Of these, radioactive labeling may be used for almost all types of assays. Enzyme-conjugated labels are particularly useful when radioactivity must be avoided or when quick results are needed. Biotin-coupled reagents usually are detected with labeled streptavidin. Streptavidin binds tightly and quickly to biotin and may be labeled with radioisotopes or enzymes. Fluorochromes, although requiring expensive equipment for their use, provide a very sensitive method of detection. Those of ordinary skill in the art will know of other suitable labels which may be employed in accordance with the present invention. The binding of these labels to antibodies or fragments thereof may be accomplished using standard techniques such as those described by Kennedy, et al. [(1976) Clin. Chim. Acta 70:1-31], and Schurs, et al. [(1977) Clin. Chim Acta 81:1-40].

In accordance with the diagnostic method of the invention, the presence or absence of the antibody-antigen complex is correlated with the presence or absence in the biological sample of the antigen, or a peptide fragment thereof. A biological sample containing elevated levels of said antigen is indicative of an invasive cancer in a subject from which the biological sample was obtained. Accordingly, the diagnostic method of the invention may be used as part of a routine screen in subjects suspected of having an invasive cancer or for subjects who may be predisposed to having an invasive cancer. Moreover, the diagnostic method of the invention may be used alone or in combination with other well-known diagnostic methods to confirm the presence of an invasive cancer.

The diagnostic method of the invention further provides that an antibody of the invention may be used to monitor the levels of GLI antigen in patient samples at various intervals of drug treatment to identify whether and to which degree the drug treatment is effective in reducing or inhibiting hyperproliferation of cells. Furthermore, antigen levels may be monitored using an antibody of the invention in studies evaluating efficacy of drug candidates in model systems and in clinical trials. The antigens provide for surrogate biomarkers in biological fluids to non-invasively assess the global status of tumor cell proliferation. For example, using an antibody of this invention, antigen levels may be monitored in biological samples of individuals treated with known or unknown therapeutic agents or toxins. This may be accomplished with cell lines in vitro or in model systems and clinical trials, depending on the cancer being investigated. Persistently increased total levels of GLI antigen in biological samples during or immediately after treatment with a drug candidate indicates that the drug candidate has little or no effect on cell proliferation. Likewise, the reduction in total levels of GLI antigen indicates that the drug candidate is effective in reducing or inhibiting tumor cell proliferation. This may provide valuable information at all stages of pre-clinical drug development, clinical drug trials as well as subsequent monitoring of patients undergoing drug treatment.

### Antibody Labels

The GLI proteins of the present invention, antibodies to GLI proteins, and nucleic acids that hybridize to gli genes (e.g. probes) etc. can all be labeled. Suitable labels include enzymes, fluorophores (e.g., fluorescein isothiocyanate (FITC), phycoerythrin (PE), Texas red (TR), rhodamine, free or chelated lanthanide series salts, especially Eu³⁺, to name a few fluorophores), chromophores, radioisotopes, chelating agents, dyes, colloidal gold, latex particles, ligands (e.g., biotin), and chemiluminescent agents. When a control marker is employed, the same or different labels may be used for the receptor and control marker.

In the instance where a radioactive label, such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I and ¹⁸⁶Re are used, known currently available counting procedures may be utilized. Such labels may also be appropriate for the nucleic acid probes used in binding studies with GLI. In the instance where the label is an enzyme, detection may be accomplished by any of the presently utilized colorimetric, spectrophotometric, fluorospectrophotometric, amperometric or gasometric techniques known in the art.

Direct labels are one example of labels which can be used according to the present invention. A direct label has been defined as an entity, which in its natural state, is readily visible, either to the naked eye, or with the aid of an optical filter and/or applied stimulation, e.g. ultraviolet light to promote fluorescence. Among examples of colored labels, which can be used according to the present invention, include metallic sol particles, for example, gold sol particles such as those described by Leuvering (U.S. Pat. No. 4,313,734); dye sole particles such as described by Gribnau et al. (U.S. Pat. No. 4,373,932) and May et al. (WO 88/08534); dyed latex such as described by May, supra, Snyder (EP-A 0 280 559 and 0 281 327); or dyes encapsulated in liposomes as described by Campbell et al. (U.S. Pat. No. 4,703,017). Other direct labels include a radionucleotide, a fluorescent moiety or a luminescent moiety. In addition to these direct labeling devices, indirect labels comprising enzymes can also be used according to the present invention. Various types of enzyme linked immunoassays are well known in the art, for example, alkaline phosphatase and horseradish peroxidase, lysozyme, glucose-6-phosphate dehydrogenase, lactate dehydrogenase, urease, these and others have been discussed in detail by Eva Engvall in Enzyme Immunoassay ELISA and EMIT in Methods in Enzymology, 70:419-439 (1980) and in U.S. Pat. No. 4,857,453.

Suitable enzymes include, but are not limited to, alkaline phosphatase and horseradish peroxidase.

In addition, GLI, a fragment thereof can be modified to contain a marker protein such as green fluorescent protein as described in U.S. Pat. No. 5,625,048 filed Apr. 29, 1997, WO 97/26333, published Jul. 24, 1997 and WO 99/64592 all of which are hereby incorporated by reference in their entireties.

Other labels for use in the invention include magnetic beads or magnetic resonance imaging labels.

In another embodiment, a phosphorylation site can be created on an antibody of the invention for labeling with ³²P, e.g., as described in European Patent No. 0372707 (application No. 89311108.8) by Sidney Pestka, or U.S. Pat. No. 5,459,240, issued Oct. 17, 1995 to Foxwell et al.

As exemplified herein, proteins, including antibodies, can be labeled by metabolic labeling. Metabolic labeling occurs during in vitro incubation of the cells that express the protein in the presence of culture medium supplemented with a metabolic label, such as [³⁵S]-methionine or [³²P]-orthophosphate. In addition to metabolic (or biosynthetic) labeling with [³⁵S]-methionine, the invention further contemplates labeling with [¹⁴C]-amino acids and [³H]-amino acids (with the tritium substituted at non-labile positions).

### Therapeutic Uses of an Antibody That Blocks the SHH-GLI Pathway

Another aspect of the invention provides that an antibody, or a fragment thereof, which blocks signaling via the SHH-GLI pathway, may be administered to a human or other animal in an amount to decrease or inhibit cell proliferation, and tumorigenesis. As one may appreciate, any hyperproliferative disorder such as cancer, which may be diagnosed by an antibody of the invention, may also be treated using an antibody of the invention. A skilled clinician or physician would be able, by routine experimentation, to determine what an effective, non-toxic amount of antibody would be for the purpose of decreasing or inhibiting cell proliferation. Generally, however, an effective dosage will be in the range of about 0.05 to 100 milligrams per kilogram body weight per day.

Furthermore, an antibody of the invention may be administered to a human or other animal in a conventional dosage form prepared by combining an antibody of the invention with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables.

The antibodies of the present invention may also be used to target other chemotherapeutic agents to the site where needed. Alternatively, the antibodies can be used to target radioisotopes to the site where inhibition of cellular proliferation is desirable. The antibodies may also be employed for diagnostic purposes to identify sites within the patient where the tumor burden is greatest. Furthermore, the antibodies may be used to assess the effectiveness of anti-tumor therapy for prognostic value.

The route of administration of an antibody, or fragment thereof, may be oral, parenteral, by inhalation or topical. The antibody may be delivered locally to the site of the cancer. The term parenteral as used herein includes intravenous, intramuscular, subcutaneous, rectal, vaginal or intraperitoneal administration. The subcutaneous and intramuscular forms of parenteral administration are generally preferred. The antibody may be delivered using a slow release formulation. It may be delivered in a liposome or a similar device.

The daily parenteral and oral dosage regimens for employing antibodies of the invention to therapeutically decrease cell proliferation will generally be in the range of about 0.05 to 100, but preferably about 0.5 to 10, milligrams per kilogram body weight per day.

An antibody of the invention may also be administered by inhalation. Inhalation, as used herein, includes intranasal and oral inhalation administration. Appropriate dosage forms for such administration, such as an aerosol formulation or a metered dose inhaler, may be prepared by conventional techniques. The preferred dosage amount of an antibody of the invention to be employed is generally within the range of about 10 to 100 milligrams.

An antibody of the invention may also be administered topically. By topical administration is meant non-systemic administration and includes the application of an antibody, or fragments thereof, externally to the epidermis, to the buccal cavity and instillation of such an antibody into the ear, eye and nose, and where it does not significantly enter the blood stream. In a particular topical formulation, the antibody may be effective in treatment of a hyperproliferative condition such as cancer. By systemic administration is meant oral, intravenous, intraperitoneal and intramuscular administration. The amount of an antibody required for therapeutic effect will, of course, vary with the antibody chosen, the nature and severity of the condition being treated and the animal undergoing treatment, and is ultimately at the discretion of the physician. A suitable topical dose of an antibody of the invention will generally be within the range of about 1 to 100 milligrams per kilogram body weight daily.

An alternate therapeutic approach for use of the antibodies of the present invention is via insertion of the gene encoding the antibody into a tumor cell whereby the intracellular expression of the antibody gene allows for modulation of the function of the protein for which the antibody is specific. Accordingly, this invention provides for methods and compositions for modulating SHH-GLI function in a cell involving intracellular expression of the antibody described herein.

The invention is particularly applicable to inhibiting the SHH-GLI signaling in a cancer cell, thus inhibiting proliferation and survival of the cell.

To express an antibody homologue within a cell, a nucleic acid molecule encoding the antibody homologue, such as a recombinant expression vector encoding the antibody homologue, is introduced into the cell. Preferably, the antibody homologue used to modulate SHH-GLI signaling is a single chain Fv (scFv) fragment, although whole antibodies, or antigen binding fragments thereof (e.g., Fab fragments) may also be useful.

In a particularly preferred embodiment of the invention, an antibody homologue is expressed intracellularly in a cancerous mammalian cell to inhibit the cell proliferation function of SHH-GLI. The target cells of interest may be selected from any cell in which SHH-GLI plays a role in proliferation, such as cancer cells. A nucleic acid molecule encoding the antibody homologue can be introduced in vivo into cells of interest, by, for example, use of a recombinant viral vector or other vector system suitable for delivery of genes to cells in vivo.

To express an antibody homologue within a cell, a nucleic acid molecule(s) encoding the antibody homologue is prepared and introduced into the cell. An isolated nucleic acid molecule encoding an antibody homologue can be prepared according to standard molecular biology methods using nucleic acid sequences obtained from antibody genes. Isolated nucleic acid molecules encoding antibody chains (or relevant antigen binding portions thereof, such as V_{H} or V_{L} regions), specific for many different particular proteins have been described, and/or are available, in the art. Additionally, such nucleic acids can be isolated by standard techniques, for example, from a hybridoma that expresses a monoclonal antibody specific for a protein of interest, or by screening an immunoglobulin expression library (e.g., an immunoglobulin phage display library) with the protein of interest.

Monoclonal antibodies specific for an antigenic polypeptide of interest may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity. Such cell lines may be produced, for example, from spleen cells obtained from an immunized animal. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against the polypeptide. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction.

Alternatively, monoclonal antibodies can be prepared by constructing a recombinant immunoglobulin library, such as a scFv or Fab phage display library and nucleic acid encoding an antibody chain (or portion thereof) can be isolated therefrom. Immunoglobulin light chain and heavy chain first strand cDNAs can be prepared from mRNA derived from lymphocytes of a subject immunized with a protein of interest using primers specific for a constant region of the heavy chain and the constant region of each of the kappa and lambda light chains. Using primers specific for the variable and constant regions, the heavy and light chain cDNAs can then by amplified by PCR. The amplified DNA is then ligated into appropriate vectors for further manipulation in generating a library of display packages. Restriction endonuclease recognition sequences may also be incorporated into the primers to allow for the cloning of the amplified fragment into a vector in a predetermined reading frame for expression on the surface of the display package.

The immunoglobulin library is expressed by a population of display packages, preferably derived from filamentous phage, to form an antibody display library. In addition to commercially available kits for generating phage display libraries (e.g., the Pharmacia Recombinant Phage Antibody System, Catalog No. 27-9400-01; and the Stratagene SurfZAP™ Phage Display Kit, Catalog No. 240612), examples of methods and reagents particularly amenable for use in generating antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J. 12:725-734; Hawkins et al. (1992) J Mol Biol 226:889-896; Clackson et al. (1991) Nature 352:624-628; Gram et al. (1992) Proc. Natl. Acad. Sci. USA 89:3576-3580; Garrad et al. (1991) Bio/Technology 2:1373-1377; Hoogenboom et al. (1991) Nuc Acid Res 19:4133-4137; and Barbas et al. (1991) Proc. Natl. Acad. Sci. USA 88:7978-7982. As generally described in McCafferty et al. Nature (1990) 348:552-554, complete VH and VL domains of an antibody, joined by a flexible (Gly₄-Ser)₃ linker, can be used to produce a single chain antibody expressed on the surface of a display package, such as a filamentous phage.

Once displayed on the surface of a display package (e.g., filamentous phage), the antibody library is screened with a protein of interest to identify and isolate packages that express an antibody that binds the protein of interest. Display packages expressing antibodies that bind immobilized protein can then be selected. Following screening and identification of a monoclonal antibody (e.g., a monoclonal scFv) specific for the protein of interest, nucleic acid encoding the selected antibody can be recovered from the display package (e.g., from the phage genome) by standard techniques. The nucleic acid so isolated can be further manipulated if desired (e.g., linked to other nucleic acid sequences) and subcloned into other expression vectors by standard recombinant DNA techniques.

Once isolated, nucleic acid molecules encoding antibody chains, or portions thereof, can be further manipulated using standard recombinant DNA techniques. For example, a single chain antibody gene can also be created by linking a VL coding region to a VH coding region via a nucleotide sequence encoding a flexible linker (e.g., (Gly₄-Ser)₃). Single chain antibodies can be engineered in accordance with the teachings of Bird et al. (1988) Science 242:423-426; Huston et al. (1988) Proc. Natl. Acad. Sci. USA 85:5879-5883; Ladner, et al. International Publication Number WO 88/06630; and McCafferty, et al. International Publication No. WO 92/10147. A preferred single chain antibody for use in the invention blocks SHH-GLI signaling. A plasmid encoding a scFv antibody would be prepared using standard molecular biological techniques.

Another manipulation that can be performed on isolated antibody genes is to link the antibody gene to a nucleotide sequence encoding an amino acid sequence that directs the antibody homologue to a particular intracellular compartment. A preferred nucleotide sequence to which an antibody gene is linked encodes a signal sequence (also referred to as a leader peptide). Signal sequences are art-recognized amino acid sequences that direct a protein containing the signal sequence at its amino-terminal end to the endoplasmic reticulum (ER). Typically, signal sequences comprise a number hydrophobic amino acid residues. Alternatively, an antibody homologue can be linked to an amino acid sequence that directs the antibody homologue to a different compartment of the cell. For example, a nuclear localization sequence (NLS) can be linked to the antibody homologue to direct the antibody homologue to the cell nucleus. Nuclear localization sequences are art-recognized targeting sequences. Typically, an NLS is composed of a number of basic amino acid residues.

Following isolation of antibody genes, as described above, and, if desired, further manipulation of the sequences, DNA encoding the antibody homologue can be inserted into an expression vector to facilitate transcription and translation of the antibody coding sequences in a host cell. Within the expression vector, the sequences encoding the antibody homologue are operatively linked to transcriptional and translational control sequences. These control sequences include promoters, enhancers and other expression control elements (e.g., polyadenylation signals). Such regulatory sequences are known to those skilled in the art and are described in Goeddel, Gene Expression Technology: Methods in Enzymology 185, Academic Press, San Diego, Calif. (1990). The expression vector and expression control sequences are chosen to be compatible with the host cell used. Expression vectors can be used to express one antibody chain (e.g., a single chain antibody) or two antibody chains (e.g., a Fab fragment). To express two antibody chains, typically the genes for both chains are inserted into the same expression vector but linked to separate control elements.

Expression of a nucleic acid in mammalian cells is accomplished using a mammalian expression vector. When used in mammalian cells, the expression vector's control functions are often provided by viral material. For example, commonly used promoters are derived from polyoma, Adenovirus 2, cytomegalovirus (CMV) and Simian Virus 40. An example of a suitable mammalian expression vector is pcDNA3 (commercially available from Invitrogen), which drives transcription via the CMV early intermediate promoter/enhancer and contains a neomycin resistance gene as a selective marker. Other examples of mammalian expression vectors include pCDM8 (Seed, B., (1987) Nature 329:840) and pMT2PC (Kaufman et al. (1987), EMBO J. 6:187-195). Alternative to the use of constitutively active viral regulatory sequences, expression of an antibody homologue gene can be controlled by a tissue-specific regulatory element that directs expression of the nucleic acid preferentially in a particular cell type. Tissue-specific regulatory elements are known in the art.

In one embodiment, a recombinant expression vector of the invention is a plasmid vector. Plasmid DNA can be introduced into cells by a variety of techniques either as naked DNA or, more commonly, as DNA complexed with or combined with another substance. Alternatively, in another embodiment, the recombinant expression vector of the invention is a virus, or portion thereof, which allows for expression of a nucleic acid introduced into the viral nucleic acid. For example, replication defective retroviruses, adenoviruses and adeno-associated viruses can be used for recombinant expression of antibody homologue genes. Virally-mediated gene transfer into cells can be accomplished by infecting the target cell with the viral vector.

Non-limiting examples of techniques which can be used to introduce an expression vector encoding an antibody homologue into a host cell include:

Adenovirus-Polylysine DNA Complexes: Naked DNA can be introduced into cells by complexing the DNA to a cation, such as polylysine, which is then coupled to the exterior of an adenovirus virion (e.g., through an antibody bridge, wherein the antibody is specific for the adenovirus molecule and the polylysine is covalently coupled to the antibody) (see Curiel, D. T., et al. (1992) Human Gene Therapy 3:147-154). Entry of the DNA into cells exploits the viral entry function, including natural disruption of endosomes to allow release of the DNA intracellularly. A particularly advantageous feature of this approach is the flexibility in the size and design of heterologous DNA that can be transferred to cells.

Receptor-Mediated DNA Uptake: Naked DNA can also be introduced into cells by complexing the DNA to a cation, such as polylysine, which is coupled to a ligand for a cell-surface receptor (see for example Wu, G. and Wu, C. H. (1988) J. Biol. Chem. 263:14621; Wilson et al. (1992) J. Biol. Chem. 267:963-967; and U.S. Pat. No. 5,166,320). Binding of the DNA-ligand complex to the receptor facilitates uptake of the DNA by receptor-mediated endocytosis. Receptors to which a DNA-ligand complex have targeted include the transferrin receptor and the asialoglycoprotein receptor. Additionally, a DNA-ligand complex can be linked to adenovirus capsids which naturally disrupt endosomes, thereby promoting release of the DNA material into the cytoplasm and avoiding degradation of the complex by intracellular lysosomes (see for example Curiel et al. (1991) Proc. Natl. Acad. Sci. USA 88:8850; and Cotten, M. et al. (1992) Proc. Natl. Acad. Sci. USA 89:6094-6098; Wagner, E. et al. (1992) Proc. Natl. Acad. Sci. USA 89:6099-6103). Receptor-mediated DNA uptake can be used to introduce DNA into cells either in vitro or in vivo and, additionally, has the added feature that DNA can be selectively targeted to a particular cell type by use of a ligand which binds to a receptor selectively expressed on a target cell of interest.

Liposome-Mediated transfection (“lipofection”): Naked DNA can be introduced into cells by mixing the DNA with a liposome suspension containing cationic lipids. The DNA/liposome complex is then incubated with cells. Liposome mediated transfection can be used to stably (or transiently) transfect cells in culture in vitro. Protocols can be found in Current Protocols in Molecular Biology, Ausubel, F. M. et al. (eds.) Greene Publishing Associates, (1989), Section 9.4 and other standard laboratory manuals. Additionally, gene delivery in vivo has been accomplished using liposomes. See for example Nicolau et al. (1987) Meth. Enz. 149:157-176; Wang and Huang (1987) Proc. Natl. Acad. Sci. USA 84:7851-7855; Brigham et al. (1989) Am. J. Med. Sci. 298:278; and Gould-Fogerite et al. (1989) Gene 84:429-438.

Direct Injection: Naked DNA can be introduced into cells by directly injecting the DNA into the cells. For an in vitro culture of cells, DNA can be introduced by microinjection, although this not practical for large numbers of cells. Direct injection has also been used to introduce naked DNA into cells in vivo (see e.g., Acsadi et al. (1991) Nature 332:815-818; Wolff et al. (1990) Science 247:1465-1468). A delivery apparatus (e.g., a “gene gun”) for injecting DNA into cells in vivo can be used. Such an apparatus is commercially available (e.g., from BioRad).

Retroviral Mediated Gene Transfer: Defective retroviruses are well characterized for use in gene transfer for gene therapy purposes (for a review see Miller, A. D. (1990) Blood 76:271). A recombinant retrovirus can be constructed having a nucleic acid encoding a gene of interest (e.g., an antibody homologue) inserted into the retroviral genome. Additionally, portions of the retroviral genome can be removed to render the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art.

Retroviruses have been used to introduce a variety of genes into many different cell types, including epithelial cells, endothelial cells, lymphocytes, myoblasts, hepatocytes, bone marrow cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573). While any retrovirus may be utilized, the lentivirus approach allows for delivery to a broad variety of cellular targets, both ex vivo (cell lines, primary cells including stem cells, fertilized oocytes, and blastocysts) and in vivo (e.g., brain, lung, liver). The lentivirus vector-mediated delivery of siRNAs allows for the controllable suppression of cellular genes both with a high degree of efficacy and without significant leakiness.

Adenoviral Mediated Gene Transfer: The genome of an adenovirus can be manipulated such that it encodes and expresses a gene product of interest (e.g., an antibody homologue) but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 d1324 or other strains of adenovirus (e.g., Ad2, Ad3, Ad7 etc.) are well known to those skilled in the art. Recombinant adenoviruses are advantageous in that they do not require dividing cells to be effective gene delivery vehicles and can be used to infect a wide variety of cell types, including airway epithelium (Rosenfeld et al. (1992) cited supra), endothelial cells (Lemarchand et al. (1992) Proc. Natl. Acad. Sci. USA 89:6482-6486), hepatocytes (Herz and Gerard (1993) Proc. Natl. Acad. Sci. USA 90:2812-2816) and muscle cells (Quantin et al. (1992) Proc. Natl. Acad. Sci. USA 89:2581-2584). Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to many other gene delivery vectors (Berkner et al. cited supra; Haj-Ahmand and Graham (1986) J. Virol. 57:267). Most replication-defective adenoviral vectors currently in use are deleted for all or parts of the viral E1 and E3 genes but retain as much as 80% of the adenoviral genetic material.

Adeno-Associated Viral Mediated Gene Transfer: Adeno-associated virus (AAV) is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of the introduced DNA can be detected, for example, by Northern blotting, RNase protection or reverse transcriptase-polymerase chain reaction (RT-PCR). Expression of the introduced gene product (e.g., the antibody homologue) in the cell can be detected by an appropriate assay for detecting proteins, for example by immunohistochemistry.

As will be appreciated by those skilled in the art, the choice of expression vector system will depend, at least in part, on the host cell targeted for introduction of the nucleic acid. For example, nucleic acid encoding an antibody homologue which blocks SHH-GLI signaling is preferably introduced into tumor cells showing enhanced proliferative capacity and highly invasive characteristics. Tumor cells that are responsive to treatment with the blocking antibodies of the present invention include prostate cancer, lung cancer (small cell and non small cell lung cancer), basal cell carcinoma, melanoma, glioblastoma, medulloblastoma, PNETs, other brain tumors, stomach, GI tract including colon, pancreas, rhabdomyosarcomas, and soft tissue sarcomas. Preferred expression vectors and delivery systems for introducing nucleic acid into malignant cells include transfection with adenoviral-polylysine DNA complexes and adenoviral vector-mediated gene transfer. These delivery systems are suitable for introduction of nucleic acid into cells in vitro, or more preferably for tumor cells, in vivo.

The functional outcome of intracellular antibody expression, on the subsequent expression and/or function of the protein targeted for antibody binding (referred to as the target protein) can be assessed by suitable assays that monitor the expression and/or function of the target protein, including standard immunohistochemistry or immunoelectron microscopy techniques.

Alternatively, cell proliferation can be measured using commercially available cell proliferation assays. The functional outcome of intracellular antibody homologue expression targeting SHH-GLI on tumor cell growth and survival, or on the expansion of immunocompetent cells with unwanted specificity in a mammal can be assessed in vivo using animal model systems that may be predictive of therapeutic efficacy in humans. For example, the antibody genes may be inserted into a human cancer cell known to have the SHH-GLI signaling pathway. These cells may be implanted into athymic nude mice, and tumor growth may be monitored visually over time.

### Other Diagnostic Means of Determining Levels of Gli

### Cell-based Reporters and Instrumentation

Cellular screening techniques can be broadly classified into two groups: semi-biochemical approaches that involve the analysis of cell lysates, or live cell assays. Whole cell assay methodologies vary with respect to assay principle, but have largely in common a form of luminescence or fluorescence for detection. Luminescence is a phenomenon in which energy is specifically channeled to a molecule to produce an excited state. Luminescence includes fluorescence, phosphorescence, chemiluminescence and bioluminescence.

An ever-increasing list of fluorescent proteins include the widely-used GFP derived from Aequorea Victoria and spectral variants thereof. The list includes a variety of fluorescent proteins derived from other marine organisms; bacteria; fungi; algae; dinoflagellates; and certain terrestrial species. These reporters have the advantage of not requiring any exogenous substrates or co-factors for the generation of a signal but do require an external source of radiation for excitation of the intrinsic fluorophore. In addition, the increasing availability of genes encoding a broad spectrum of fluorescent reporter proteins enables the construction of assays tailored for specific applications, cell types, and detection systems.

Different classes of luminescent proteins, luciferases, have been have been discovered in bacteria and eukaryotes. Luciferases are proteins that catalyze the conversion of a natural substrate into a product that emits light in the visible spectrum and thus require no external radiation source. Monomeric forms of luciferase have been cloned from firefly, Renilla, and other organisms. Firefly luciferase is the most common of the bioluminescent reporters and is a 61 kDa monomeric enzyme that catalyzes a two-step oxidation reaction to yield light. Renilla luciferase is a 31 kDa monomeric enzyme that catalyzes the oxidation of coelenterazine to yield coelenteramide and blue light of 480 nm. Substrates for luciferase are widely available from commercial suppliers such as Promega Corporation and Invitrogen Molecular Probes.

A variety of useful enzymatic reporters are enzymes that either generate a fluorescent signal or are capable of binding small molecules that can be tagged with a fluorescent moiety to serve as a fluorescent probe. For example, dihydrofolate reductase (DHFR) is capable of binding methotrexate with high affinity; a methotrexate-fluorophore conjugate can serve as a quantitative fluorescent reagent for the measurement of the amount of DHFR within a cell. By tagging methotrexate with any of a number of fluorescent molecules such as fluorescein, rhodamine, Texas Red, BODIPY and other commercially available molecules (such as those available from Molecular Probes/Invitrogen and other suppliers) a range variety of fluorescent readouts can be generated. The wide range of techniques of immunohistochemistry and immunocytochemistry can be applied to whole cells. For example, ligands and other probes can be tagged directly with fluorescein or another fluorophore for detection of binding to cellular proteins; or can be tagged with enzymes such as alkaline phosphatase or horseradish peroxidase to enable indirect detection and localization of signal.

Many other enzymes can be used to generate a fluorescent signal in live cells by using specific, cell-permeable substrate that either becomes fluorescent or shifts its fluorescence spectrum upon enzymatic cleavage. For example, substrates for beta-lactamase exist whose fluorescence emission properties change in a measurable way upon cleavage of a beta-lactam core moiety to which fluorophores are attached. Changes include, shifts in fluorophore absorption or emission wavelengths, or cleavage of a covalent assembly of emmision-absorption-mathched fluorophore pairs that in the covalently-assembled form sustain resonance energy transfer between the two fluorophores that is lost when the two are separated. Membrane-permeant, fluorescent BLA substrates such as the widely-used CCF2/AM allow the measurement of gene expression in live mammalian cells in the absence or presence of compounds from a biologically active chemical library.

Luminescent, fluorescent or bioluminescent signals are easily detected and quantified with any one of a variety of automated and/or high-throughput instrumentation systems including fluorescence multi-well plate readers, fluorescence activated cell sorters (FACS) and automated cell-based imaging systems that provide spatial resolution of the signal. A variety of instrumentation systems have been developed to automate HCS including the automated fluorescence imaging and automated microscopy systems developed by Cellomics, Amersham, TTP, Q3DM, Evotec, Universal Imaging and Zeiss. Fluorescence recovery after photobleaching (FRAP) and time lapse fluorescence microscopy have also been used to study protein mobility in living cells. Although the optical instrumentation and hardware have advanced to the point that any bioluminescent signal can be detected with high sensitivity and high throughput, the existing assay choices are limited either with respect to their range of application, format, biological relevance, or ease of use.

### Transcriptional Reporter Assays

Cell-based reporters are often used to construct transcriptional reporter assays, which allow monitoring of the cellular events associated with signal transduction and gene expression. Reporter gene assays couple the biological activity of a target to the expression of a readily detected enzyme or protein reporter. Based upon the fusion of transcriptional control elements to a variety of reporter genes, these systems “report” the effects of a cascade of signaling events on gene expression inside cells. Synthetic repeats of a particular response element can be inserted upstream of the reporter gene to regulate its expression in response to signaling molecules generated by activation of a specific pathway in a live cell. The variety of transcriptional reporter genes and their application is very broad and includes drug screening systems based on beta-galactosidase (beta-gal), luciferase, alkaline phosphatase (luminescent assay), GFP, aequorin, and a variety of newer bioluminescent or fluorescent reporters.

In general, transcription reporter assays have the capacity to provide information on the response of a pathway to natural or synthetic chemical agents on one or more biochemical pathways, however they only indirectly measure the effect of an agent on a pathway by measuring the consequence of pathway activation or inhibition, and not the site of action of the compound. For this reason, mammalian cell-based methods have been sought to directly quantitate protein-protein interactions that comprise the functional elements of cellular biochemical pathways and to develop assays for drug discovery based on these pathways.

Cellular Assays for Individual Proteins Tagged with Fluorophores or Luminophores.

Subcellular compartmentalization of signaling proteins is an important phenomenon not only in defining how a biochemical pathway is activated but also in influencing the desired physiological consequence of pathway activation. This aspect of drug discovery has seen a major advance as a result of the cloning and availability of a variety of intrinsically fluorescent proteins with distinct molecular properties.

High-content (also known as high-context) screening (HCS) is a live cell assay approach that relies upon image-based analysis of cells to detect the subcellular location and redistribution of proteins in response to stimuli or inhibitors of cellular processes. Fluorescent probes can be used in HCS; for example, receptor internalization can be measured using a fluorescently-labeled ligand that binds to the transferrin receptor. Often, individual proteins are either expressed as fusion proteins, where the protein of interest is fused to a detectable moiety such as GFP, or are detected by immunocytochemistry after fixation, such as by the use of an antibody conjugated to Cy3 or another suitable dye. In this way, the subcellular location of a protein can be imaged and tracked in real time. One of the largest areas of development is in applications of GFP color-shifted mutants and other more recently isolated new fluorescent proteins, which allow the development of increasingly advanced live cell assays such as multi-color assays. A range of GFP assays have been developed to analyze key intracellular signaling pathways by following the redistribution of GFP fusion proteins in live cells. For drug screening by HCS the objective is to identify therapeutic compounds that block disease pathways by inhibiting the movement of key signaling proteins to their site of action within the cell.

Tagging a protein with a fluorophore or a luminophore enables tracking of that particular protein in response to cell stimuli or inhibitors. For example, the activation of cell signaling by TNF can be detected by expressing the p65 subunit of the NFkB transcription complex as a GFP fusion and then following the redistribution of fluorescence from the cytosolic compartment to the nuclear compartment of the cell within minutes after TNF stimulation of live cells (JA Schmid et al., 2000, Dynamics of NFkB and IkBa studied with green fluorescent protein (GFP) fusion proteins, J. Biol. Chem. 275: 17035-17042). What has been unique about these approaches is the ability to allow monitoring of the dynamics of individual protein movements in living cells, thus addressing both the spatial and temporal aspects of signaling.

### Gene Therapy and Transgenic Vectors

A gene encoding a hedgehog protein or a GLI protein, e.g., SHH or GLI1, GLI2 or GLI3, an active fragment thereof, derivative thereof, or structural/functional domain thereof, can be introduced either in vivo, ex vivo, or in vitro in a viral vector as noted previously.

In a particular embodiment, an appropriate immunosuppressive treatment is employed in conjunction with the viral vector, e.g., adenovirus vector, to avoid immuno-deactivation of the viral vector and transfected cells. For example, immunosuppressive cytokines, such as interleukin-12 (IL-12), interferon, or anti-CD4 antibody, can be administered to block humoral or cellular immune responses to the viral vectors (see, e.g., Wilson, Nature Medicine, (1995)). In addition, it is advantageous to employ a viral vector that is engineered to express a minimal number of antigens.

In another embodiment, and as noted above, the Shh or gli gene can be introduced in a retroviral vector, e.g., as described in U.S. Pat. No. 5,399,346; Mann et al., (1983) Cell, 33:153; U.S. Pat. No. 4,650,764; U.S. Pat. No. 4,980,289; Markowitz et al., (1988) J. Virol., 62:1120; U.S. Pat. No. 5,124,263; International Patent Publication No. WO 95/07358, published Mar. 16, 1995; and Kuo et al., (1993) Blood, 82:845.

Targeted gene delivery is described in International Patent Publication WO 95/28494, published October 1995.

Alternatively, the vector can be introduced by lipofection. Liposomes may be used for encapsulation and transfection of nucleic acids in vitro. Synthetic cationic lipids designed to limit the difficulties and dangers encountered with liposome mediated transfection can be used to prepare liposomes for in vivo transfection of a gene encoding SHH (Feigner, et. al., Proc. Natl. Acad. Sci. U.S.A., 84:7413-7417 (1987); see Mackey, et al., Proc. Natl. Acad. Sci. U.S.A., 85:8027-8031 (1988)). The use of cationic lipids may promote encapsulation of negatively charged nucleic acids, and also promote fusion with negatively charged cell membranes (Feigner and Ringold, Science, 337:387-388 (1989)). The use of lipofection to introduce exogenous genes into the specific organs in vivo has certain practical advantages. Molecular targeting of liposomes to specific cells represents one area of benefit. It is clear that directing transfection to particular cell types would be particularly advantageous in a tissue with cellular heterogeneity, such as the brain. Lipids may be chemically coupled to other molecules for the purpose of targeting (see Mackey et. al., Proc. Natl. Acad. Sci. U.S.A., 85:8027-8031 (1988)).

It is also possible to introduce the vector as a naked DNA plasmid. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun, or use of a DNA vector transporter (see, e.g., Wu et al., (1992) J. Biol. Chem., 267:963-967; Wu and Wu, (1988) J. Biol. Chem., 263:14621-14624; Hartmut et al., Canadian Patent Application No. 2,012,311, filed Mar. 15, 1990).

In a preferred embodiment of the present invention, a gene therapy vector as described above employs a transcription control sequence operably associated with the nucleotide sequence encoding the SHH inserted in the vector. That is, a specific expression vector of the present invention can be used in gene therapy.

Such an expression vector is particularly useful to regulate expression of a therapeutic hh gene, e.g., sonic hedgehog gene. In one embodiment, the present invention contemplates constitutive expression of the hh gene, even if at low levels. Alternatively, a regulatable promoter may be used.

### Administration

According to the present invention, a therapeutic composition, e.g., an SHH or GLI protein or active fragment thereof and a pharmaceutically acceptable carrier of the invention or an agent such as a small organic molecule that stimulates the SHH-GLI pathway and/or increases expression of SHH, or alternatively, a small organic molecule or an siRNA or blocking antibody to block or inhibit the SHH-GLI pathway to prevent tumorigenesis, may be introduced parenterally, transmucosally, e.g., nasally. Preferably, administration is by intracranial, intrathecal or intraventricular administration. Alternatively, the therapeutic composition can be placed (e.g., injected) into the bloodstream after coupling the SHH or GLI protein or active fragment thereof to a carrier that will allow the SHH or GLI protein or active fragment thereof—carrier complex to cross the blood-brain barrier.

In a preferred aspect, an HH or GLI protein of the present invention can cross cellular or nuclear membranes, which would allow for intravenous or oral administration. Strategies are available for such crossing, including but not limited to, increasing the hydrophobic nature of a molecule; introducing the molecule as a conjugate to a carrier, such as a ligand to a specific receptor, targeted to a receptor; and the like.

The present invention also provides for conjugating targeting molecules to an HH or GLI protein. “Targeting molecule” as used herein shall mean a molecule which, when administered in vivo, localizes to desired location(s). In various embodiments, the targeting molecule can be a peptide or protein, antibody, lectin, carbohydrate, or steroid. In one embodiment, the targeting molecule is a peptide ligand of a receptor on the target cell. (On the other hand SHH may itself be considered a targeting molecule since it binds its own receptor). In a specific embodiment, the targeting molecule is an antibody. Preferably, the targeting molecule is a monoclonal antibody. In one embodiment, to facilitate crosslinking the antibody can be reduced to two heavy and light chain heterodimers, or the F(ab′)₂ fragment can be reduced, and crosslinked to the SHH protein via a reduced sulfhydryl.

Antibodies for use as targeting molecule are specific for cell surface antigen. In one embodiment, the antigen is a receptor. For example, an antibody specific for a receptor on a brain stem cell can be used. This invention further provides for the use of other targeting molecules, such as lectins, carbohydrates, proteins and steroids.

In another embodiment, the therapeutic compound can be delivered in a vesicle, in particular a liposome (see Langer, (1990) Science, 249:1527-1533; Treat et al., (1989) in Liposomes in the Therapy of Infectious Disease and Cancer, Lopez-Berestein and Fidler (eds.), Liss: New York, pp. 353-365; Lopez-Berestein, ibid., pp. 317-327; see generally ibid.).

In yet another embodiment, the therapeutic compound can be delivered in a controlled release system. For example, the polypeptide may be administered using intravenous infusion, an implantable osmotic pump, a transdermal patch, liposomes, or other modes of administration. In one embodiment, a pump may be used (see Langer (1990) supra; Sefton, (1987) CRC Crit. Ref Biomed. Eng., 14:201; Buchwald et al., (1980) Surgery, 88:507; Saudek et al., (1989) N. Engl. J. Med., 321:574). In another embodiment, polymeric materials can be used (see Medical Applications of Controlled Release, Langer and Wise (eds.), CRC Press: Boca Raton, Fla. (1974); Controlled Drug Bioavailability, Drug Product Design and Performance, Smolen and Ball (eds.), Wiley: New York (1984); Ranger and Peppas, (1983) J. Macromol. Sci. Rev. Macromol. Chem., 23:61; see also Levy et al., (1985) Science, 228:190; During et al., (1989) Ann. Neurol., 25:351; Howard et al., (1989) J. Neurosurg., 71:105). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)). Preferably, a controlled release device is introduced into a subject in proximity of the adult brain stem cells, e.g., the striatal subventricular zone (SVZ). Other controlled release systems are discussed in the review by Langer (1990) supra.

### Specific Embodiments

As shown below the inventors have not only examined the role of GLI proteins in carcinogenesis, in particular prostate cancer, but have explored the effects of Gli1 on apoptosis induced by various chemotherapeutic agents. The inventors have provided evidence to support a role for GLI in tumorigenesis and tumor cell proliferation and more importantly from a clinical point of view, in resistance to cell killing by cancer chemotherapeutic agents.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### SHH-GLI Signaling and Tumorigenesis

Animals

Swiss-Webster mice were used unless otherwise specified. The Shh (Chiang et al., (1996) Nature 383, 407-413), Gli1 (Park et al., (2000) Development 127, 1593-1605) and Gli2 (Mo et al., (1997) Development 124, 113-123.) mutants from our colony were in this background. Cortical explants were prepared as previously described (Dahmane et al., (2001) Development 128, 5201-5212) and treated for 48 h. Anti-SHH antibodies were used at 4 μg/ml (University of Iowa Hybridoma Bank). Octyl-modified SHH-N protein was a kind gift from Ontogeny/Curis Inc. Human tumor samples were derived from the operating room or from the NYU tumor banks. Brain tumor cell lines were obtained from ATCC and grown according to its specifications. GL261 was a kind gift of Dr D. Zagzag. Frog (Xenopus laevis) embryos were obtained, reared and staged by standard methods. Tadpoles were ˜2 days old. All statistical analyses were carried out using the Student's t test and deviations are shown as s.e.m.

### In Vivo Cyclopamine Treatment

Cyclopamine (Toronto Research Biochemicals) was used at 1 mg/ml conjugated with 2-Hydropropyl-β-Cyclodextrin (HBC (Sigma); prepared as a 45% solution in PBS). Five to ten week-old inbred C57B16/j mice were injected intraperitoneally for one week with HBC alone as control or cyc at 10 mg/kg/day. The day following the last injection, the mice were pulsed for 2 h with BrdU (20 mg/kg, IP injection). Immunofluorescence of cryostat sections was as described (Dahmane et al., (2001 Development 128, 5201-5212). The stainings were digitally recorded using a cooled CCD camera-equiped Axiophot (Zeiss) and the BrdU⁺/DAPI⁺ nuclei counted within the lateral wall of the lateral ventricles. For the in vivo treatment followed by the preparation of nsps, pregnant mothers (E12.5) or P4 pups were injected for 5 days with HBC alone or cyc at 10 mg/kg/day and cortical nsp from E17.5 embryos or SVZ nsp from P9 animals were made, respectively.

Cell Culture and siRNAs Transfections

U87 glioblastoma cells were cultured in MEM with 10% fetal bovine serum (FBS), and glioblastoma primary cultures were cultured in DMEM/F12 with 10% FBS. For transfection of siRNAs, cells were plated the day before the treatment in p16 well plates, at a 70% cell density. 24 h later cells were transfected using the Oligofectamine reagent from Gibco (cat#12252-011) following the specifications of the manufacturer. The final concentration of siRNA was 200 nM. Three hours after, the transfection media was changed to normal growing media (10% FBS) and the cells were kept growing for another 48 h (or 24 h where indicated) before processing. The following sequences were used for the siRNA experiments:

(SEQ ID NO: 21)
human GLI1 AACTCCACAGGCATACAGGAT
(SEQ ID NO: 22)
human/mouse GLI2 AAGATCTGGACAGGGATGACT
(SEQ ID NO: 23)
mouse GLI2 AATGATCTCTGCCGCCAGGGG
(SEQ ID NO: 24)
human/mouse GLI3 AATGAGGATGAAAGTCCTGGA

### Proliferation Assay

BrdU (6 μg/ml) incorporation was done for 2 h in U87 glioblastoma cells and for 14 h in the primary glioblastoma cells. After that the cells were fixed for 5 minutes in paraformaldehide (PFA) 4%. Immunocytochemistry was performed with an anti-BrdU antibody (Becton-Dickinson) and fluorescein-conjugated secondary antibodies (Boehringer Mannheim). The measurement was done by counting percentage of BrdU positive cells per field, counting at least 8 fields per point.

### Explants, Dissociated Cells, Cell Treatments and Chemicals

Neocortical or tectal explants from embryonic day (E) 17.5 to postnatal-day (P) 3 mice were taken from the parietal region, or adjacent to the dorsal midline in the prospective superior and inferior colliculi, respectively. After removal of the meninges, the explants were grown on floating filters in serum-free media. After 12 hours in culture, SHH was added and incubation continued for a further 48 hours. Explants for RNA preparation were directly collected in Trizol (Gibco-BRL). For dissociated cells, parietal cortical pieces of P3 brains were pooled (˜10 explants per experiment) and treated with trypsin for 10 minutes (0.25 mg/ml at room temperature). Tissue was triturated manually in DNase (0.5 mg/ml), and cells were centrifuged, resuspended in supplemented serum-free media and plated at a density of ˜400 cells/mm² in poly-L-lysine-coated 16- or 8-well slides. After 12 hours the media were replaced and SHH protein added if required. Cells were cultured for a further 48 hours and processed for immunocytochemistry after fixation in 4% paraformaldehyde for 1 minute. Primary gliomas from the operating room were dissociated with papain and plated in U118 media containing 10% fetal calf serum (FCS) or in DMEM:F12 serum-free media supplemented with BIT-9500 (Stem Cell Technologies) and 20 ng/ml of each of FGF2, EGF and PDGF. After two to three passages, the cells had a homogenous appearance and were then tested. Recombinant N-SHH was a kind gift from Ontogeny and was used at 5 nM. For blocking experiments, anti-SHH mAb 5E1 was used at 20 μg/ml (obtained from the University of Iowa Hybridoma Bank). Cyclopamine (a kind gift from the Poisonous Plant Laboratory or purchased from Toronto Research Chemicals) was used at 0.5-5 μM for 48 hours before assaying. Cell lines were plated at 60% confluency the night before cyclopamine treatment. FK and ddFK (Sigma) were used at 50 μM.

### Microinjection, RNAs and Antisense Oligonucleotides

Injection of synthetic RNAs into frog embryos was performed into one cell at the two-cell stage, targeting the future CNS and epidermis. Frog or human Gli1 RNAs were injected at 2 ng/10 nl/embryo. The N-terminal Myc-epitope tag in the frog Gli1 and Gli2 proteins was used to monitor protein distribution. lacZ RNA was co-injected at 0.2 ng/10 nl/embryo and was used as a lineage tracer through X-gal staining, yielding an insoluble blue precipitate. Morpholino antisense oligonucleotides were purchased from Gene Tools and used at 0.5 mM. These were frog Gli1, 5′CGGGCGGACACTGGCGGGACGC3′ (SEQ ID NO: 33); frog Gli2, 5′GCACAGAACGCAGGTAATGCTCCAT3′ (SEQ ID NO: 34); and frog Shh, 5′GAGATTCGAGTTCGCAACCAGCATC3′ (Seq ID NO: 35). In all cases, the oligonucleotides were designed to be complementary to regions near the initiation ATG codon and are predicted to inhibit translation.

### RT-PCR and In Situ Hybridization

In situ hybridization on serial ˜20 μm cryostat sections with digoxigenin-labeled antisense RNA probes and full-length frog, mouse or human Gli1 cDNA clones and histology were as previously described (Dahmane et al. (1997), Nature 389:876-881). Visualization of the low levels of GLI1 and PTCH1 expression in CNS tumors and mouse brains older than E17 required long (2 days at room temperature) chromogenic development of the in situ hybridization reactions. Specificity was confirmed using sense RNA probes. RT-PCR of human tumors or cell lines was performed for 27, 32 and 37 cycles to determine the linear amplification range. PCR primers and specific reaction conditions are available upon request. A probe for DDR1 was made as described (Weiner et al. (2000), Neurosurgery 47: 1400-1409). A 0.6 kb RT-PCR clone was used as a template for probe production for mouse Pdgfrα (platelet-derived growth factor receptor α).

### Immunocytochemistry

BrdU incorporation in explants and dissociated cells was for 2 hours at 6 μg/ml. Primary tumor cultures were labelled with BrdU for 14 hours. Pregnant mice were injected intraperitoneally with a single dose of 50 μl of 10 mg/ml BrdU and embryos dissected 2 hours afterwards. For tadpoles, one 20 n1 injection of 10 mg/ml BrdU into the lumen of the CNS and one into the endoderm were performed 1 hour before fixation. Sections of embryos or explants (14-20 μm) were prepared using a cryostat. Immunocytochemistry with monoclonal anti-BrdU antibody (Becton-Dickinson), monoclonal anti-vimentin antibody (Santa Cruz), monoclonal anti-neuronal tubulin Tuj 1 antibody (Babco), rat monoclonal anti-Nestin antibody (University of Iowa Hybridoma Bank) or O4 monoclonal antibody (Chemicon, also a kind gift of Bob Miller) was performed on frozen sections and cells using fluorescein-conjugated secondary antibodies (Boehringer Mannheim).

### GLI Gene Expression in Primary Brain Tumors and Brain Tumor Cell Lines

Our findings in normal development raise the possibility that inappropriate activation or maintenance of the SHH-GLI pathway could lead to hyperproliferation, the basis of tumorigenesis. To test this idea, sporadic human brain tumors were analyzed for the consistent expression of the GLI genes. We tested by RT-PCR seven glial tumors and primitive neuroectodermal tumors (PNETs), including those from the cerebellum (medulloblastomas), because the latter have been shown previously to harbor PTCH1 mutations, suggesting the activation of the SHH-GLI pathway. We found that all the tumors tested expressed GLI1, although at different levels (data not shown). Additional analyses for a total of 22 tumors showed that all samples contained the three GLI transcripts. Expression of PTCH1 followed the expression of GLI1/2, while that of SHH was not consistently detected.

In situ hybridization analyses of 22 independent brain tumors showed that GM was expressed in neuronal and in glial tumors, including glioblastoma multiforme and low grade glioma. GLI1 and PTCH1 mRNAs were detected in the regions containing tumor cells and not in surrounding normal tissues.

Analyses of human brain tumor cell lines, including seven glioblastoma (U87MG, U118MG, U138MG, A172, T98G, M059K, M059J), two glioma (Hs683, mouse GL261), one neuroglioma (H4), three astrocytoma (CCF-STTG1, SW1088, SW1783), three medulloblastoma (Daoy, D283, D341) and two neuroblastoma (SK-N-AS, IMR32) lines, showed that all brain tumor cell lines co-expressed GLI1, GLI2 and PTCH1. GLI3 was expressed by all but one (D341) and only a subset (U87MG, U138MG, Daoy, M059K, SW1783) expressed SHH (not shown).

As a control, a panel of unrelated sporadic human tumors was also tested by RT-PCR and found that GLI1 was expressed consistently in prostate carcinomas (9/11 cases) but not in those from the breast (1/7), suggesting that prostate cancer may also result from deregulated SHH-GLI signaling.

### Cyclopamine Modulates the Proliferation of a Subset of Brain Tumor Cells

Expression of the GLI and PTCH1 by glioma cells raised the possibility that these harbor mutations that activate the pathway at different levels. Indeed, we expected that only a fraction of these possible mutations would affect the PTCH-SMO receptor complex. To address this possibility we have tested the effects of cyclopamine, a drug that inhibits the function of oncogenic Smoothened forms. The glioblastoma/glioma lines U87, U118, U138, M059K, Hs683, C6, GL261, astrocytoma lines, SW1088 and SW1783, and the medulloblastoma line Daoy were tested and four responded to cyclopamine treatment by decreasing BrdU incorporation by ˜25-50%. These are the glioma lines U87, U118 and U138, and the medulloblastoma line Daoy: untreated U87, 22.5±1.4% BrdU-positive cells/field; 0.5 μM cyclopamine treated, 16.5±0.9% BrdU-positive cells/field, P<0.005; and 5 μM cyclopamine-treated, 13.2±1.2% BrdU-positive cells/field, P<0.001; untreated U118, 12.9±1.3% BrdU-positive cells/field; 0.5 μM cyclopamine treated, 6.7±0.8% BrdU-positive cells/field, P=0.001; and 5 μM cyclopamine treated, 7.1±1.1% BrdU-positive cells/field, P<0.005; untreated U138, 13.3±0.7% BrdU-positive cells/field; 0.5 μM cyclopamine treated, 9.5±0.6% BrdU-positive cells/field, P<0.005; and 5 μM cyclopamine-treated, 7.5±1.2% BrdU-positive cells/field, P=0.001; untreated Daoy, 37.9±2% BrdU-positive cells/field; 0.5 μM cyclopamine treated, 27.7±1.5% BrdU-positive cells/field, P=0.001; and 5 μM cyclopamine-treated, 27.1±1.7% BrdU-positive cells/field, P=0.001. While it is unclear why these four lines respond differently, these results show that their proliferation is modulated by cyclopamine-sensitive targets. Non-responsive cells could have mutations that affect the activation of the pathway downstream of the receptor complex.

Cyclopamine was also tested in three primary cortical gliomas that were dissociated and cultured in vitro. Dividing cells from all three tumors expressed vimentin (not shown), which marks neural precursors in culture among other cell types. These cells also expressed GLI1 and GLI2 but not Shh. Treatment with 5 μM cyclopamine resulted in the inhibition of BrdU incorporation in one of them by ˜60% (untreated tumor 3, 11.4±1.5% BrdU-positive cells/field; cyclopamine treated, 3.6±0.5% BrdU-positive cells/field, P<0.001), while the other two were unresponsive (untreated tumor 4, 4.5±0.5% BrdU-positive cells/field; treated, 4.6±0.5% BrdU-positive cells/field, P>0.9; and untreated tumor 5, 2.4±0.7% BrdU-positive cells/field; treated, 3.2±0.3% BrdU-positive cells/field, P=0.3).

Deregulated GLI1 Function is Sufficient to Induce Hyperproliferation of CNS Cells with Precursor Character

The results with brain tumors and cell lines suggest that the deregulated SHH-GLI pathway may be involved in abnormal proliferation. To directly test this idea, we have misexpressed Gli1 in the CNS of the developing frog embryo. Tadpoles expressing Gli1 after unilateral injections developed ipsilateral neural tube hyperplasias (24/36 embryos), first detected at tailbud stages, that expressed the β-gal lineage tracer (15/15 embryos). Most hyperplasias appeared in the hindbrain and spinal cord, consistent with the more frequent distribution of the injected materials in these areas, and showed an increase in the number of BrdU-positive cells (more than fivefold: 19±1 BrdU-positive cells on average per control neural tube side in three sections counted of independent control embryos and 91±5.6 BrdU-positive cells per injected neural tube side in three sections of independent Gli1-injected tadpoles; P<0.005) when compared with the normal, uninjected contralateral side where BrdU-positive cells were confined to the vz zone. As expected, abnormal tissue contained HNF-3β-positive floor plate cells and neurons but a large proportion of the hyperplastic masses had an undifferentiated appearance. In cases where the injected Gli1 RNA localized to the epidermis, the resulting skin hyperplasias or BCC-like tumors also showed a marked increase in the number of BrdU-positive cells (not shown) over that seen in the normal, contralateral epidermis. Gli2 or Gli3 did not have these effects because they induced ectopic mesoderm earlier).

The expression of Gli1 in glial tumors, together with the involvement of the SHH-GLI pathway in the development of glial lineages, raised the possibility that Gli1-induced hyperplasias could contain glial precursors. Pdgfrα labels oligodendrocyte and other precursors) and is a marker of gliomas). In tadpoles, Pdgfrα is first expressed within the CNS in a small group of bilateral cells of the central vz of the diencephalon, overlapping Gli1 expression (not shown). Gli1-injected tadpoles showed ectopic expression of Pdgfrα within the hyperplasias 20/45). Control uninjected tadpoles (0/60) failed to show ectopic expression of Pdgfrα.

To test if these hyperplasias include precursor cells with vz character, we have used the expression of endogenous Gli1 as a marker, as this gene is only transiently expressed in vz cells in normal development. Sectioning human GLI1 RNA-injected embryos after in situ hybridization with a frog Gli1 probe (which does not cross-hybridize with the injected, exogenous human RNA under the conditions used) showed that the majority (12/16 embryos) had unilateral hyperplasias ectopically expressing endogenous Gli1 .

### Activation of Endogenous Gli1 Function is Required for Hyperproliferation

The expression of endogenous GLI1 in tumors and in induced tadpole hyperplasias raised the possibility that its function is required in tumor formation. This could be consistent with the observation that abnormal growths in the CNS or epidermis were first detected towards the late neurula-early tailbud stages, when most of the injected material had already been degraded. To test if endogenous Gli1 is required for tumor development, we injected human GLI1 RNA along with a morpholino antisense oligonucleotide specific for the endogenous frog Gli1 mRNA that does not recognize the injected human GLI1 RNA. Injection of human GLI1 and lacZ RNAs resulted in the development of both CNS and skin hyperplasias (35/36). By contrast, co-injection of these same RNAs plus morpholino anti-frog Gli1 resulted in normal development without tumor formation. This effect is specific, as morpholino anti-frog Gli2 (n=47/52), morpholino anti-frog Shh (n=48/50) or a control unrelated morpholino (n=49/49) did not prevent tumor development by co-injected human GLI1 (not shown).

### SUMMARY OF RESULTS

The results showed that both the U87 glioblastoma cells, as well as the primary glioblastoma cells, when transfected with small inhibitory RNA molecules specific for Gli1, showed significant inhibition in proliferative capacity, as compared to control cells.

Shh-producing cells are located in the neocortical and tectal plates, in the dentate gyrus, in the Purkinje (PL) layer and at the lower levels in germinative zones. Responding cells are located in the ventricular/subventricular zones (VZ/SVZ) of the neocortex and tectum, in the external germinal layer (EGL) of the cerebellum and the subgranular layer of the hippocampus.

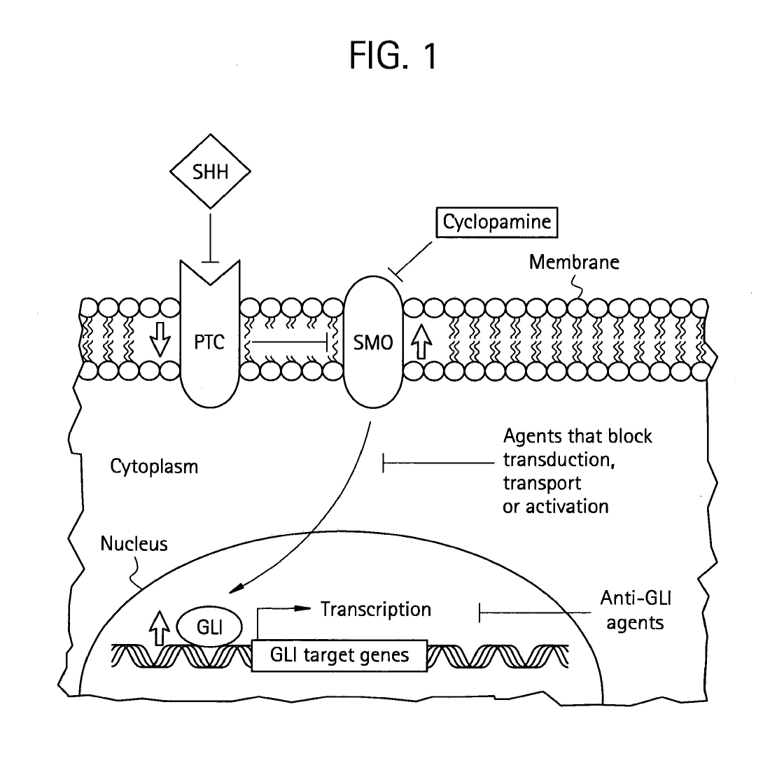

FIG. 1 shows the SHH-GLI pathway and the potential sites for therapeutic agents blocking its activity. Inhibitors of the pathway with potential therapeutic value include agents that inhibit the action of SMO (Smoothened) in the receptor complex, such as plant alkaloid cyclopamine, agents that inhibit specific aspects of the transduction of the signal, including the nuclear import or activation of Gli proteins, and agents that specifically inhibit Gli function.

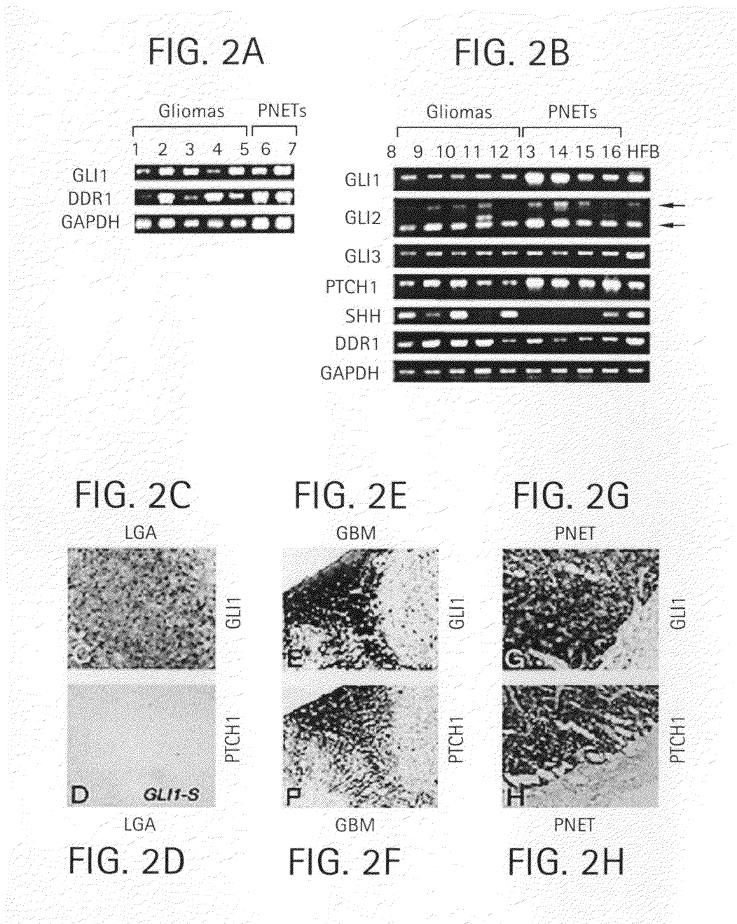

Gli genes are consistently expressed in primary brain tumors (FIG. 2) (A,B) RT-PCR analyses of independent brain tumor samples, in particular, in PNET: primitive neuroectodermal tumors; (C-H) In situ hybridization of two cortical gliomas (GBM, glioblastoma multiforme (C,D); LGA, low grade astrocytoma (E,F) and a cerebellar PNET, medulloblastoma.

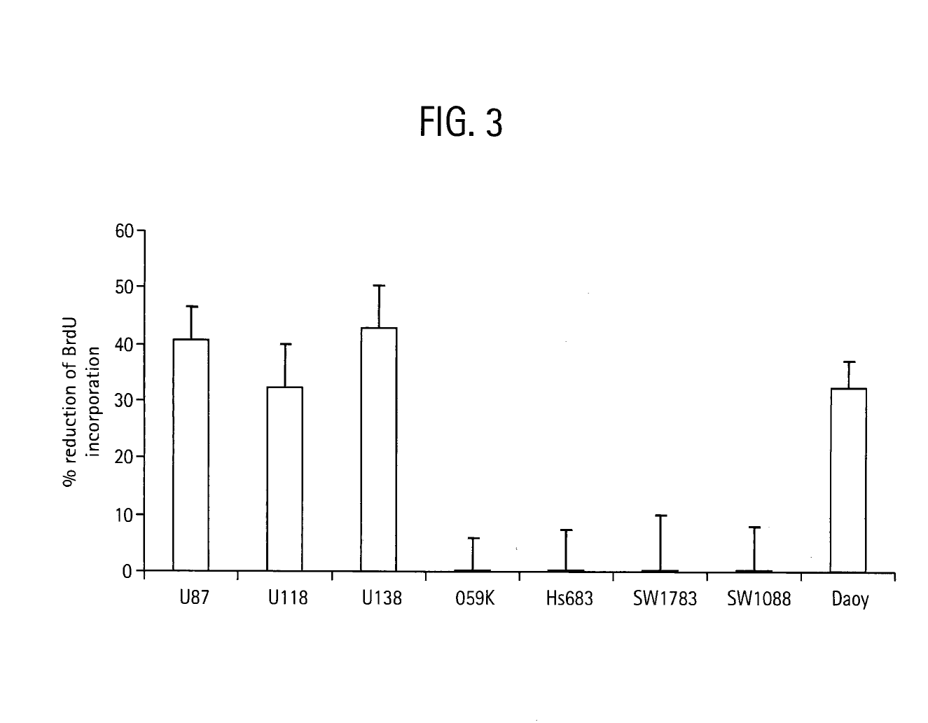

Furthermore, cyclopamine, a drug that inhibits the response to Shh signaling, modulates the proliferation of a subset of brain tumor cell lines at a concentration of 10 μM in 2.5% serum, as measured by a decrease in BrdU incorporation compared to carrier treated cells, in Glioblastoma multiforme: U87, U118, U138, 059K; in Glioma: Hs683, SW1783, SW1088; and in Medulloblastoma Daoy (FIG. 3).

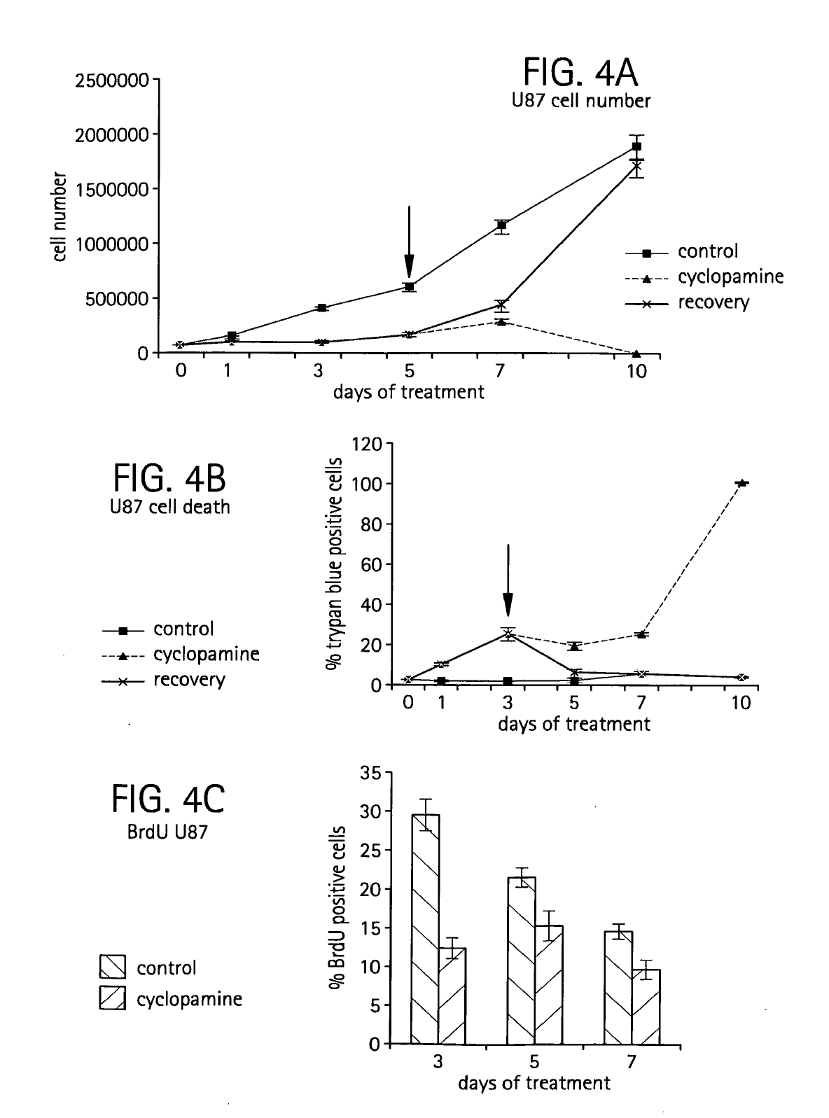

In addition, FIG. 4 demonstrates the effect of cyclopamine in long-term treatment of a glioblastoma cell line (U87) in vitro. Shh-Gli pathway controls the proliferation and viability of brain tumor cells. (A, C) shows that the proliferation of the U87 (glioblastoma) cell line is inhibited by treatment with cyclopamine (20 μM) in the presence of 10% serum (note that serum partially inhibits cyclopamine action). The cells recover upon retirement of the drug (indicated by the bar) showing that the drug is not cytotoxic. (B) A long term treatment of U87 induces death of 100% of the cells in culture.

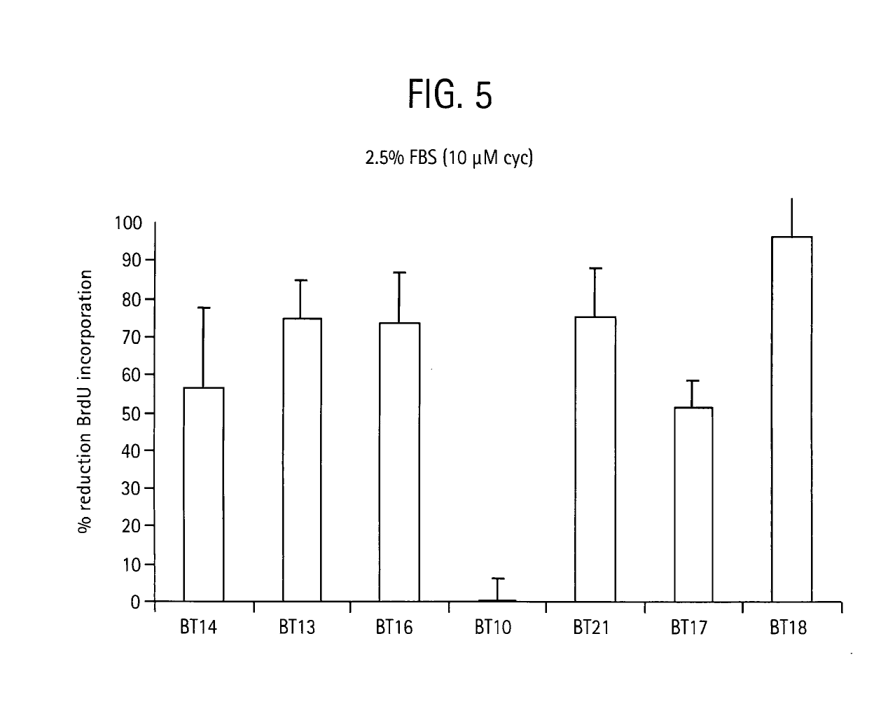

Furthermore, FIG. 5 demonstrates that cyclopamine modulates the proliferation of primary cortical gliomas that are dissociated and cultured in vitro. Inhibition of primary cortical gliomas treated with 10 μM of cyclopamine (in 2.5% serum) was measured by a decrease in BrdU incorporation in the carrier treated cells.

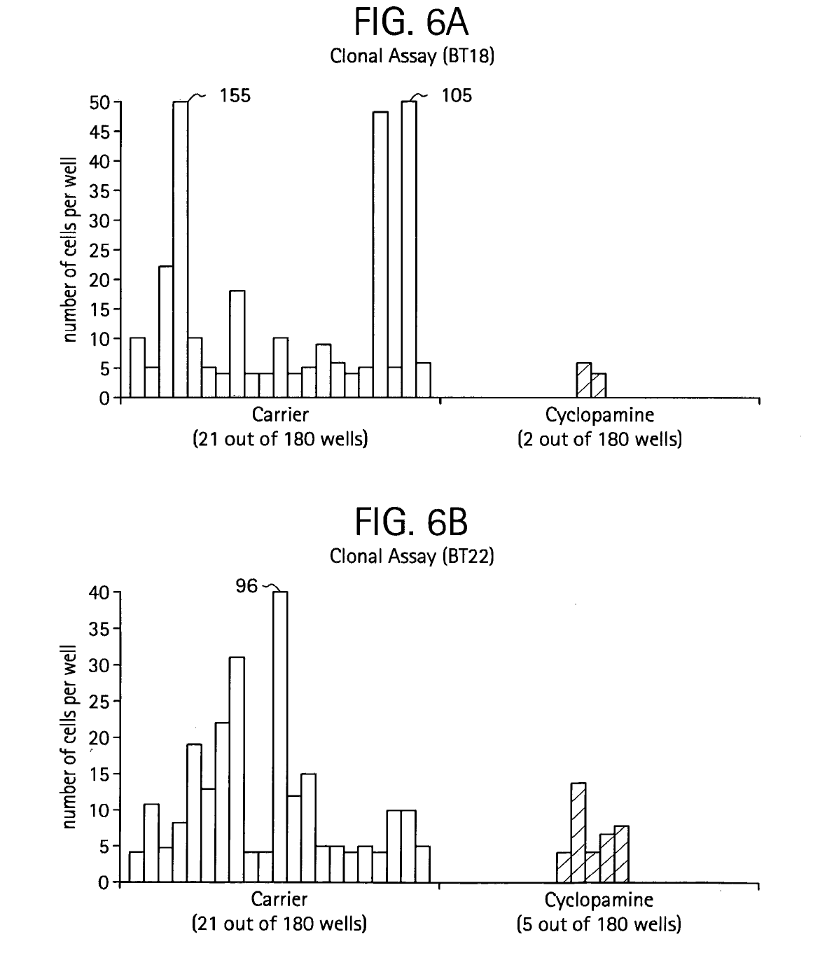

In addition, FIG. 6 demonstrates that only a subset of cells from primary brain tumors, dissociated and cultured in vitro, have stem-like properties, and their proliferation is inhibited by the presence of cyclopamine. In particular, a small number of cells from two primary brain gliomas, BT18 (top panel) and BT22 (bottom panel) are able to produce clones when plated at a density of 1 cell per well. The size and the percentage of clones are inhibited in the presence of 10 μM of cyclopamine (in 10% serum). The graphs represent the number of cells per well after 2 weeks of culture.

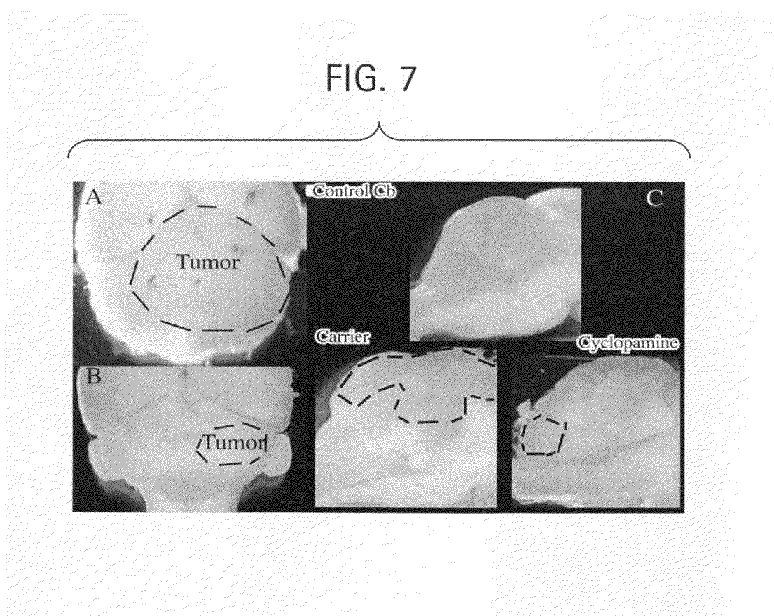

FIG. 7 demonstrates that in vivo cyclopamine treatment reduces the size of medulloblastomas of Ptch+/−, p53−/− mice. Animals were treated for a month with either cyclodextrin (carrier) (A) or cyclopamine (B). At two months of age the animals were sacrificed and the brains analyzed. (C) Sagital views of dissected cerebella. The tumors are demarcated by dashed lines.

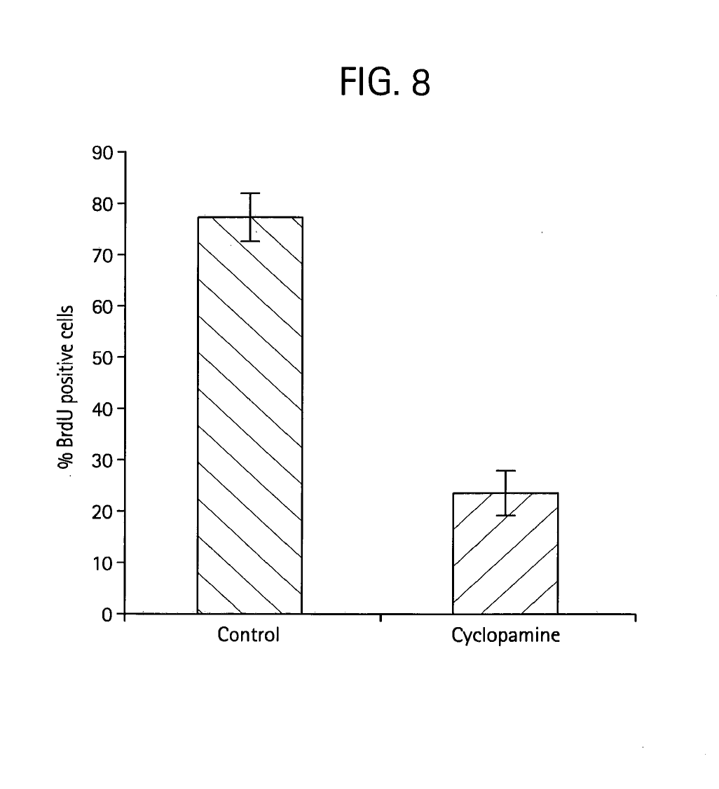

Medulloblastomas were dissected from affected mice and the cells plated for culture after dissection. FIG. 8 shows the percentage of BrdU incorporation in the presence of absence of 5 μM cyclopamine (with no serum). As shown in the figure, cyclopamine treatment resulted in a significant decrease in BrdU incorporation.

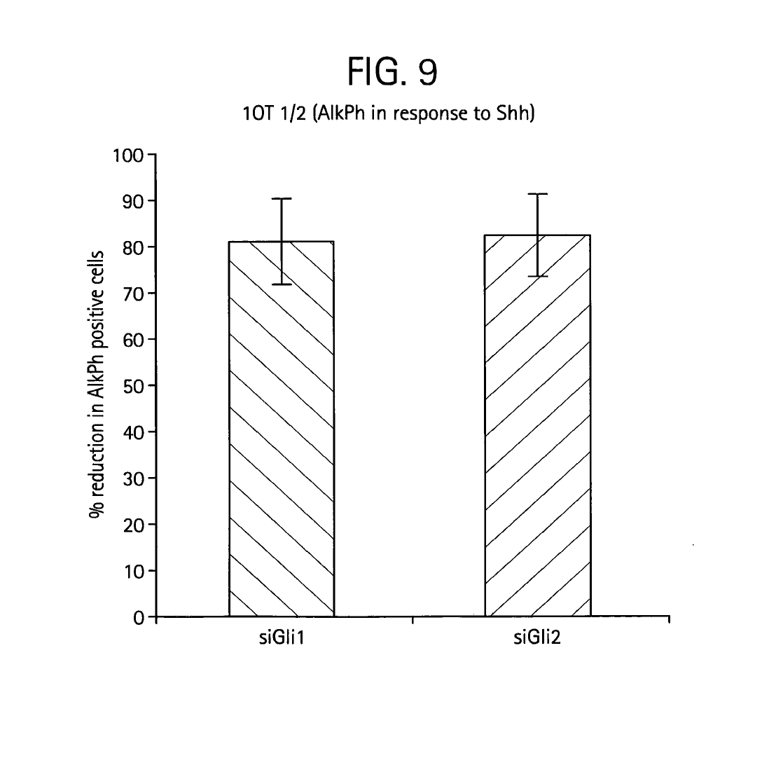

FIG. 9 demonstrates that siRNAs for Gli 1 and Gli 2 block Shh responses in 10T1/2 cells. In particular, Shh induces alkaline phosphatase expression in 10T1/2 cells. The results show about 80% reduction in inhibition of alkaline phosphatase expression in the presence of siRNAs for Gli 1 or Gli2 vs controls.

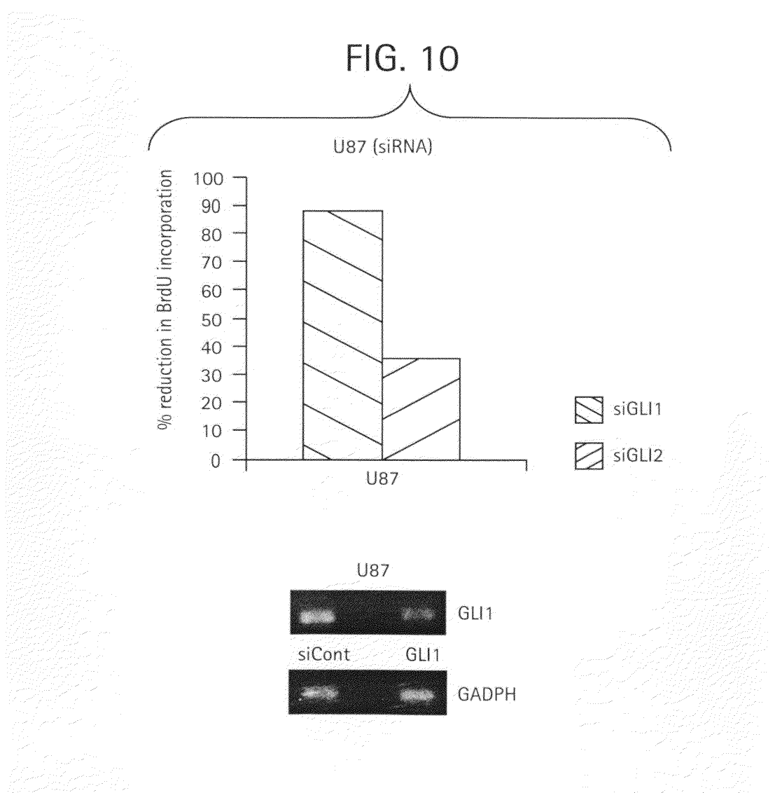

In addition, FIG. 10 shows that downregulation of Gli 1 and Gli 2 inhibits U87 glioma cell proliferation. In particular, BrdU incorporation in U87 cells was measured in cells that were transfected with either siControl, siGli1 or siGli2. The left panel shows the percentage of inhibition of cell growth in the cells treated with siGli1/2 vs the control. The right panel shows the decrease in Gli1 messenger after siGli transfection (measured by RT-PCR).

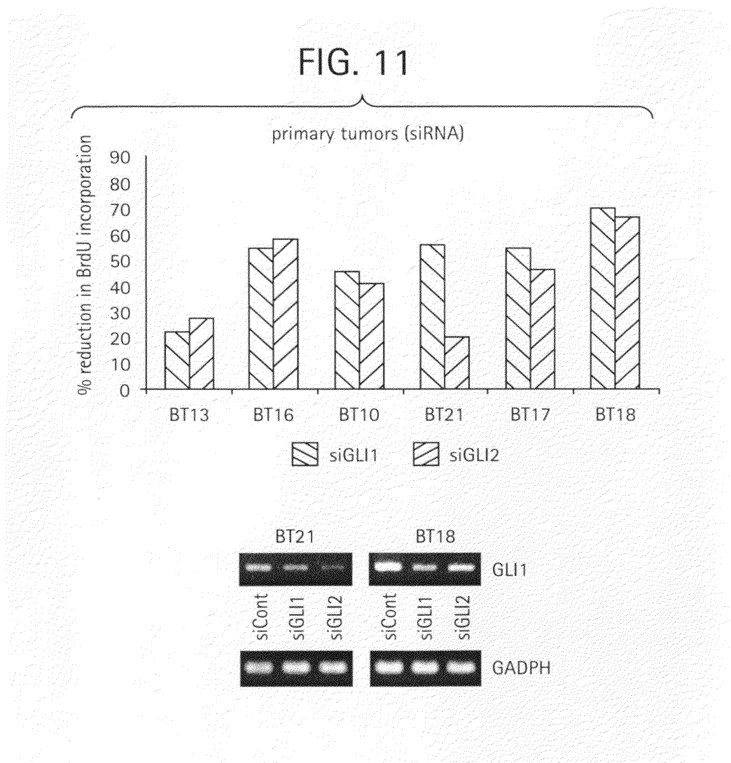

FIG. 11 shows that the proliferation of primary brain tumor cells is inhibited by blocking Gli1 and Gli2. The top panel shows the percentage of inhibition of proliferation (measured by BrdU incorporation) of several primary brain tumor cells in the presence of RNA interference for Gli1 or Gli2 vs the control. The bottom panel shows the decrease in Gli 1 messenger which reflects the inhibition of the SHH-Gli1 pathway after transfection of Gli1 or Gli2 siRNAs.

### Example 2

### Inhibition of Prostate Cancer by Interference with Sonic Hedgehog-Gli1 Signaling

### Materials and Methods

### Cell Lines and Primary Cultures

The PC3, LNCaP and DU145 cell lines (Stone, K. R., Mickey, D. D., Wunderli, H., Mickey, G. H. & Paulson, D. F. (1978) Int. J. Cancer 21, 274-281 (1978); Kaighn, M. E., Lechner J. F., Narayan K. S. & Jones L. W. (1978) Natl. Cancer Inst. Monogr. 49, 17-21; Horoszewicz, J. S., Leong, S. S., Chu, T. M., Wajsman, Z. L., Friedman, M., Papsidero, L., Kim, U., Chai, L. S., Kakati, S., Arya, S. K. & Sandberg, A. A. (1980) Prog. Clin. Biol. Res. 37, 115-132) were purchased from ATCC and grown as specified. All primary prostate tumors were obtained following approved protocols. Tumors in PBS were chopped with a razor blade and incubated with Papain for 1 h at 37 degrees, they were then dissociated by passing them through a fire polished pipette and washed several times in serum containing media. All dissociated primary tumors were plated in polyornithin-laminin treated p16 plates in DMEM-F12 with 10% FBS at ˜30,000 cells/p16 well. Primary cultures were used 2-4 days after plating, when the cells reached 60-70% confluence.

### In Situ by Hybridization and Immunocytochemistry

Immunocytochemistry was performed with anti-BrdU (R&D), anti-SHH (Santa Cruz) anti-Ki-67 (DAKO), using FITC- or HRP-conjugated secondary antibodies (Boehringer Mannheim) as described (Dahmane, N., Sanchez, P., Gitton, Y., Palma, V., Sun, T., Beyna, M., Weiner, H. & Ruiz i Altaba, A. (2001). Development 128, 5201-5212). For tissue arrays, slides were baked and deparaffinized prior to blocking of endogenous peroxides. They were then developed with HRP conjugated secondary antibodies and DAB. In situ hybridizations on frozen sections with digoxygenin-labeled antisense RNA probes for GLI1, PTCH1, SHH and a sense control GLI1 probes were as described (Dahmane, N., Sanchez, P., Gitton, Y., Palma, V., Sun, T., Beyna, M., Weiner, H. & Ruiz i Altaba, A. (2001). Development 128, 5201-5212).

### Prostate Tissue Microarrays and Microdissection

After institutional review board approval, tissue microarrays (Rhim, J. S. (2000) Prostate Cancer Prostatic Dis. 3, 229-235) were prepared from archived paraffin blocks from 288 radical prostatectomy cases from the Medical College of Wisconsin. For each case 0.6 mm cores of tumor were isolated and placed in the array blocks, and 5 um slides were prepared for immunohistochemistry. Slides were reviewed (by a trained urologic pathologist, MWD) and scored for the presence of benign prostate glands, high-grade prostatic intraepithelial neoplasia, or invasive tumor. The presence of tumor or high-grade prostatic intraepithelial neoplasia was confirmed by immunohistochemical staining for high molecular weight cytokeratin (CK903 Ab, (DAKO). Individual cores were examined as duplicates and staining correlated to a set of anonymous de-identified pathologic and outcomes data, with Chi-squared, Fisher's Exact or two-tailed ANOVA analyses.

Normal and tumor tissue from the same patients for real time PCR analyses were microdissected from sections with a laser capture microscope following pathological assessment.

SHH, anti-SHH Antibody, Cyclopamine and Tomatidine Treatments

Commercial N-SHH(R&D) was used at 100 nM since we have found that this commercial protein is ˜20× less active than the octyl-modified SHH-N we had previously used from Curis in the C3H10T1/2 induction assay (not shown). 5E1 anti-SHH blocking antibody (Ericson J, Morton S, Kawakami A, Roelink H, Jessell T M. (1996) Cell 87, 661-673) was purchased from the Hybridoma Bank at the University of Iowa and was used at 8 μg/ml. Cyclopamine (Toronto Bio.) and Tomatidine (Sigma) were used at 10 μM unless otherwise noted. For cells in culture they were dissolved in ethanol and ethanol alone used as control. Treated cells were in 2.5% serum for 48 h instead of the usual 10% routinely used for standard growth.

### Proliferation Assays

BrdU (Sigma) was given at 4 μg/ml prior to fixation. The time of the BrdU pulse depended on the growth rate of the cells tested. Cell lines were given a 2 h pulse while primary tumor cultures, which grow less rapidly, were given 16 h pulses. Proliferation in tissue arrays was measured by the level of Ki-67 antigen expression.

### Polymerase Chain Reactions

For RT-PCR reactions the following primers were used all 5′->3′: GLI1s:

### GGGATGATCCCACATCCTCAGTC (SEQ ID NO: 25) and GLI1a:

### CTGGAGCAGCCCCCCCAGT (SEQ ID NO: 26) at 60° C.; PSAs:

### CTTGTAGCCTCTCGTGGCAG (SEQ ID NO: 27) and PSAa:

GACCTTCATAGCATCCGTGAG (SEQ ID NO: 28) at 56° C. Primers for PTCH1 and GAPDH were as described (Dahmane, N., Sanchez, P., Gitton, Y., Palma, V., Sun, T., Beyna, M., Weiner, H. & Ruiz i Altaba, A. (2001). Development 128, 5201-5212; Palma, V. & Ruiz i Altaba, A. (2004) Development. 131, 337-345).

For real-time PCR, total RNA was DNAse treated (Invitrogen) and reverse transcribed with Taqman (Applied Biosystems) using oligo-dT primers as described by the manufacturer. Reactions were run using SYBER Green (Applied Biosystems) on an ABI Prism 7700 machine. Each sample was run minimally at three concentrations in triplicate. All primer sets amplified 75-300 bp fragments. Sequences are available upon request. The raw data is available upon request from S.D.

### RNA Interference

21 nt-long double stranded siRNAs were purchased from Dahrmacon Inc. purified and desalted. The sequence for the GLI1 siRNA used was: AACUCCACAGGCAUACAGGAU (SEQ ID NO: 29). SEQ ID NO: 29 corresponds to base pair numbers 685-705 of NM_{—}005269 (SEQ ID NO: 15). The sequence for the GLI2 siRNA tested was: AAGAUCUGGACAGGGAUGACU (SEQ ID NO: 31). SEQ ID NO: 31 corresponds to base pair numbers 792-812 of AB007296 ((SEQ ID NO: 17). The sequence for the GLI3 siRNA tested was AAUGAGGAUGAAAGUCCUGGA (SEQ ID NO: 32). The sequence of control siRNA was: AACGUACGCGGAAUACAACGA (SEQ ID NO: 30). This siRNA was also used FITC-tagged. SiRNA transfections (0.2 μM) were with Oligofectamine (Invitrogen, Inc) as described by the manufacturer. Cells were treated for 60 h before fixation.

### Results

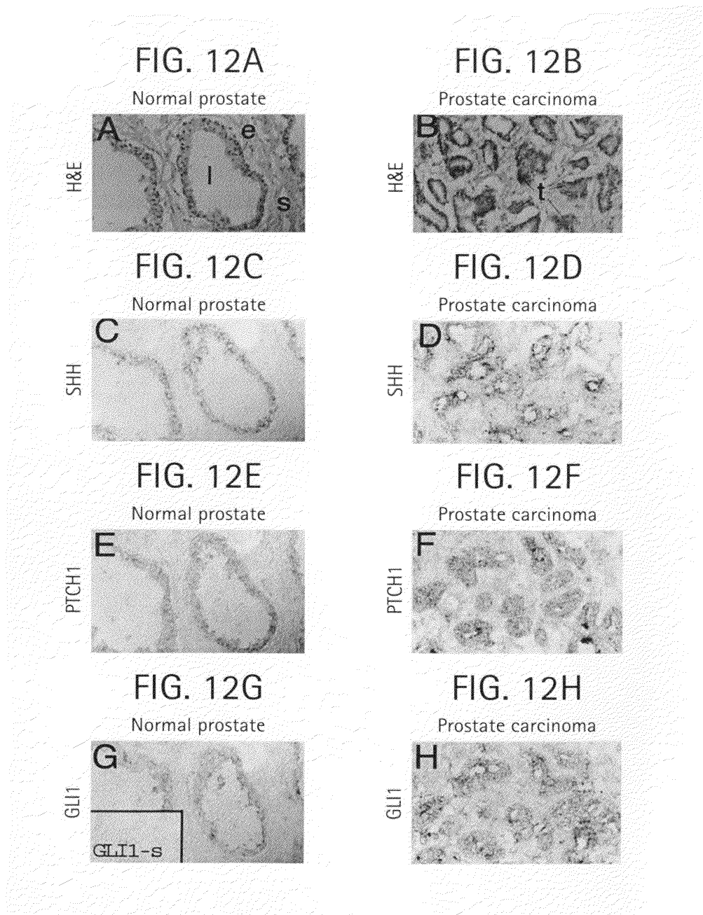

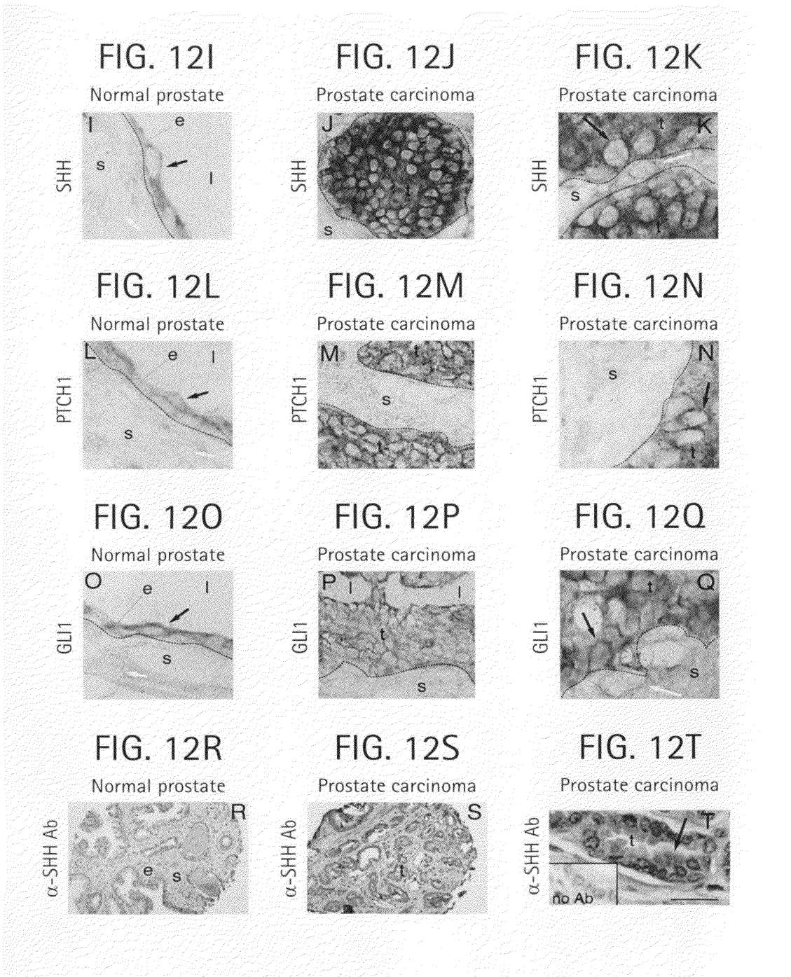

To begin to analyze the role of SHH-GLI signaling in prostate cancer the inventors first tested for the expression of SHH-GLI pathway components in prostate cancer resections and normal tissue from the same patients. In situ hybridization showed that GLI1, PTCH1 and SHH are normally co-expressed in epithelial cells and not in the surrounding stroma (FIG. 12 A, C, E, G, I, L, O). Prostate tumors were uniformly SHH⁺/GLI1⁺/PTCH1 (FIG. 12 B, D, F, H, J, K, M, N, P, Q) although variable levels of expression were detected visually in the tumors (FIG. 12). Co-expression of these markers is consistent with the derivation of the tumors from the normal prostatic epithelium.

More sensitive real-time PCR analyses of 6 of the same microdissected matched pairs showed upregulation of the expression of SHH, PTCH1, GLI1, GLI2 and GLI3 (between 1.5 to ˜300 fold) in many tumor cases compared to normal tissue after normalization to the ubiquitous similar expression of beta actin (Table 1B). Levels of expression in tumors were variable. Such differences could be related to the known heterogeneity of prostate cancer, as this is a general diagnosis that encompasses a broad range of histological phenotypes (Bostwick, D. G., Shan, A., Qian, J., Darson, M., Maihle, N. J., Jenkins, R. B., Cheng, L. (1998) Cancer 83, 1995-2002; Kaplan-Lefko, P. J., Chen, T. M., Ittmann, M. M., Barrios, R. J., Ayala, G. E., Huss, W. J., Maddison, L. A., Foster, B. A., Greenberg, N. M. (2003) Prostate 55, 219-237; DeMarzo, A. M., Nelson, W. G., Isaacs, W. B. & Epstein, J. I. (2003) Lancet. 361, 955-64). While varying levels have also been observed in other tumors, the meaning of such differences is not known, although they have been proposed to correlate in a direct or inverse manner with tumor type or grade (Pomeroy, S. L., Tamayo, P., Gaasenbeek, M., Sturla, L. M., Angelo, M., McLaughlin, M. E., Kim, J. Y., Goumnerova, L. C., Black, P. M., Lau, C. et al. (2002) Nature. 415, 436-42; Grachtchouk V, Grachtchouk M, Lowe L, Johnson T, Wei L, Wang A, de Sauvage F, Dlugosz A A. (2003) EMBO J. 22, 2741-51; Katayam M, Yoshida K, Ishimori H, Katayama M, Kawase T, Motoyama J, Kamiguchi H. (2002) J. Neurooncol. 59, 107-15). What is important is that the loyal markers of an active SHH-GLI pathway, GLI1 and PTCH1 (Lee J, Platt K A, Censullo P, & Ruiz i Altaba A. (1997) Development 124, 2537-2552; Hynes M, Stone D M, Dowd M, Pitts-Meek S, Goddard A, Gurney A, Rosenthal A. (1997) Neuron 19, 15-26; Goodrich, L. V., Johnson, R. L., Milenkovic, L., McMahon, J. A. & Scott, M. P. (1996) Genes Dev. 10, 301-12), are consistently transcribed in the examined tumor cells, showing the presence of an active pathway.

To extend these findings the inventors performed immunohistochemistry for SHH, as a secreted and potentially useful systemic marker for prostate cancer, on tissue microarrays representing 239 prostate carcinomas, 15 precancerous lesion high-grade prostatic intraepithelial neoplasia (HGPIN), and 135 benign prostate tissues from 297 patients. SHH expression was increased in tumors and was present as a secreted protein in the glandular lumens made by tumor cells (FIG. 12 K-M), likely reflecting the origin of tumors from the SHH⁺ prostatic epithelia. Higher SHH levels, determined visually, were found in 33% of tumors compared to <1% of cases of normal adjacent tissue, indicating a significant correlation between high SHH levels and tumor presence. High SHH levels were also correlated with higher Ki-67⁺ cell proliferation (Table 2). The level of SHH expression was not correlated with Gleason score or other clinical parameters (Table 2). This may indicate that inappropriately maintained or elevated SHH expression is an early and general event in prostate cancer, reflecting the origin of tumors from the SHH⁺ prostatic epithelia.

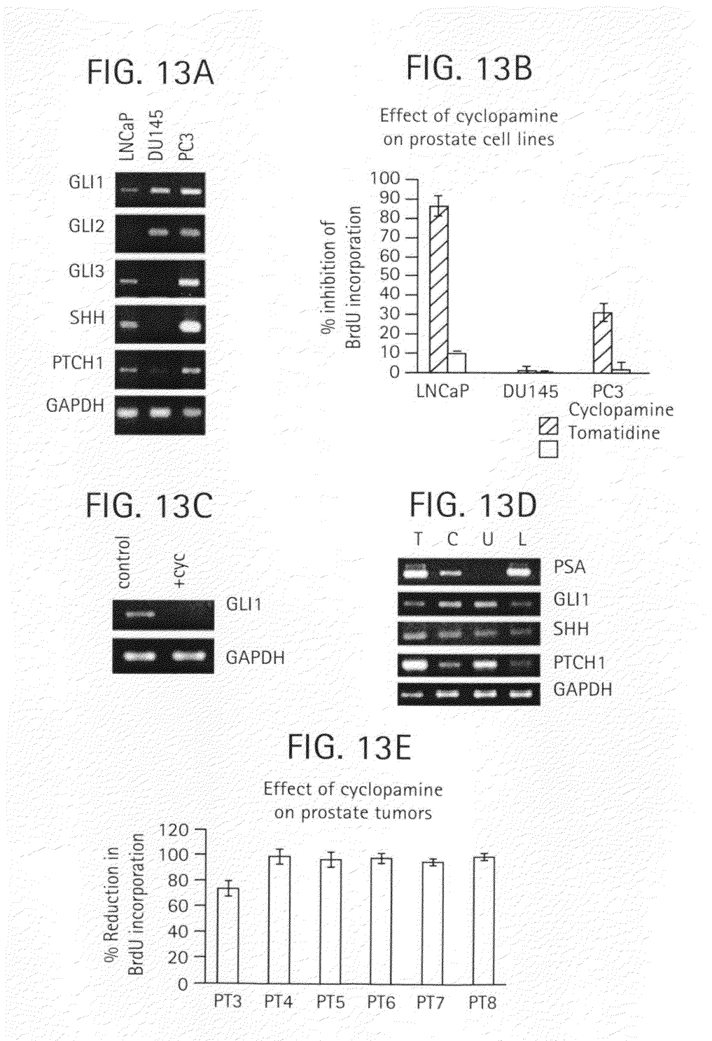

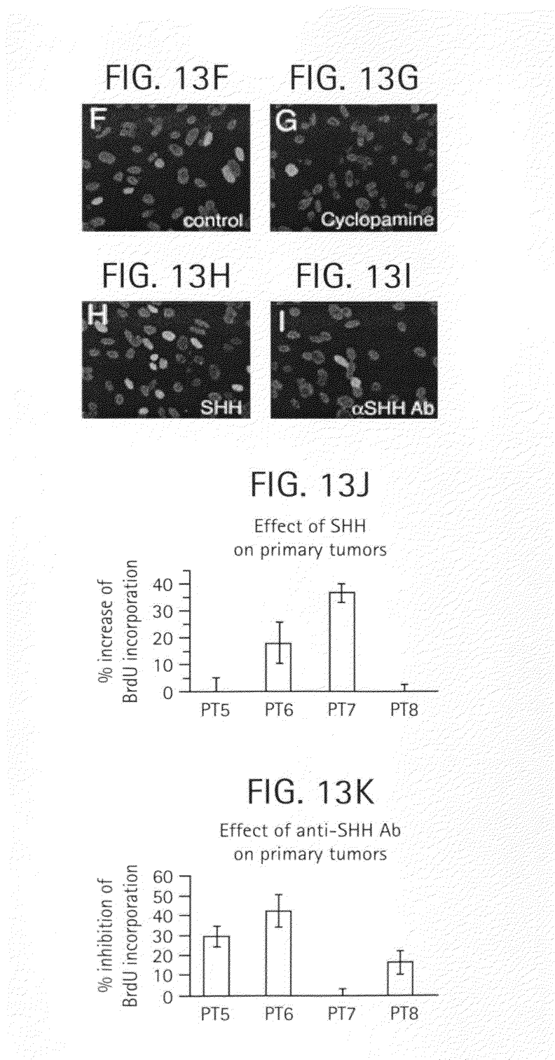

The difficulty of growing human prostate cancer cells in vitro translates into a dearth of available cancer cells to test. Here the inventors have chosen the three most widely used prostate cancer cell lines—LNCaP, an androgen sensitive cell line derived from a prostate cancer lymph node metastasis; and PC3 and DU145, androgen insensitive cell lines derived from prostate cancer bone metastases—to assay for the expression of SHH-GLI pathway components. All the cells expressed GLI1 and PTCH1 (FIG. 13A), consistent with the expression studies and indicating that they harbor an active pathway. Of these cell lines, only DU145 and PC3 cells expressed GLI2, and only LNCaP and PC3 cells expressed GL13, and SHH, at detectable levels (FIG. 13A). GLI1 is thus the only GLI gene consistently expressed at detectable levels in all of these cells and here the inventors have thus focused on GLI1.

In order to interfere with SHH-GLI signaling the inventors first used cyclopamine, a selective inhibitor of SMOH (Chen, J. K., Taipale, J., Cooper, M. K. & Beachy, P. A. (2002) Genes Dev. 16, 2743-2748). Effects of cyclopamine treatment after 48 h were tested by BrdU incorporation as a sensitive measure of cell proliferation. Such treatment led to a large (>80%) decrease in BrdU incorporation in LNCaP cells, and a significant decrease (˜30%) in PC3 cells but had no effect in DU145 cells (FIG. 13B). Treatment with tomatidine (Chen, J. K., Taipale, J., Cooper, M. K. & Beachy, P. A. (2002) Genes Dev. 16, 2743-2748) served as control and had little or no effect on BrdU incorporation (FIG. 13B). The lack of effects of cyclopamine on DU145 cells shows that this drug is not non-specific. Because the inventors used short-term assays to focus on early, direct effects on cell proliferation, the changes in total cell number were consequently relatively conservative. For instance, cyclopamine reduced total DAPI⁺ LNCaP cell number by 22.1+/−1.1% (p=0.0001) after 48 h. No cytotoxic effects or significant cell death were observed during these experiments. Cyclopamine treatment also led to a decrease in GLI1 expression, consistent with the expected down-regulation of the SHH-GLI pathway (FIG. 13C).

Analyses of primary prostate tumors is complicated by the difficulty of growing primary human prostate cancer cultures (Matysiak, B. E., Brodzeller, T., Buck, S., French, A., Counts, C., Boorsma, B., Datta, M. W. & Kajdacsy-Balla, A. A. (2003). Appl. Immunohistochem. Mol. Morphol. 11, 269-273). Nevertheless, we were able to dissociate and plate 6/8 primary prostate tumors although stable cultures were not obtained. Primary cells that remained attached after 2 days had a uniform cuboidal morphology, formed small clusters and expressed Prostate specific antigen (PSA), as well as SHH, PTCH1 and GLI1 (FIG. 13D), proving their prostatic epithelial origin. Cyclopamine treatment led to a major (>70%) decrease in BrdU incorporation in all primary cultures as compared with carrier-treated samples (FIG. 13E-G), mimicking the results obtained in LNCaP cells. Here again, the insensitivity of DU145 to cyclopamine provides a control for the action of the drug. Indeed, while the response of normal human prostate cells to cyclopamine has not yet been tested, the inventors expect that it would also inhibit the proliferation of such normal SHH⁺/PTCH1⁺/GLI1⁺ prostate epithelial cells (FIG. 12). As with the cell lines, the total number of DAPI⁺ primary tumor cells was similarly reduced by cyclopamine treatment (e.g. 26.7+/−1.1% decrease in primary tumor 6 (PT6), p=0.001) after 48 h. While stromal cells are likely to be present in our primary cultures, their numbers appear to be small as >90% of the cells examined microscopically had a similar cuboidal morphology. Moreover, the high inhibition levels by cyclopamine would be inconsistent with effects only in contaminating stromal cells, which do not appreciably express PTCH1 or GLI1 (FIG. 12).

The inventors then tested for the ability of exogenous SHH to stimulate prostate cancer cell proliferation and for the possible existence of autocrine signaling. Addition of recombinant SHH protein led to an increase in BrdU incorporation in 2/4 primary cultures after 48 h (FIG. 13 F, H, J). In contrast, addition of the standard blocking antibody against SHH (5E1; ref. 29) resulted in a inhibition of BrdU incorporation by 15-40% for 3/4 tumors (FIG. 13F, I, K), suggesting that several tumors display autocrine signaling. Interestingly the only primary culture that was insensitive to Shh Ab blockade, PT7, being sensitive to cyclopamine (which targets SMOH, FIG. 13E), was also the more sensitive to the addition of exogenous Shh. This might indicate that while the pathway is activated downstream of the site of ligand action in P1′7, possibly affecting PTCH1 or SMOH, exogenous Shh can still increase the levels of signaling. Taken together, the functional heterogeneity that was detected parallels that found for GLI and SHH expression described above and may reflect independent activating events as well as the well-known heterogeneity of prostate cancers.

Treatment of LNCaP, PC3 or DU145 cells with either blocking antibody or recombinant Shh protein did not result in significant changes in BrdU incorporation (not shown). LNCaP and PC3 cells could thus display an activated pathway at the membrane level (being sensitive to cyclopamine inhibition) that has lost responsiveness to ligand. Cyclopamine-insensitive DU145 cells may have an activated pathway downstream of SMOH (or at the level of SMOH affecting its inhibition by cyclopamine), having lost also the ability to respond to SHH. It remains possible that the different behavior of primary cultures versus established cell lines also reflects unrelated transformation or immortalization events.

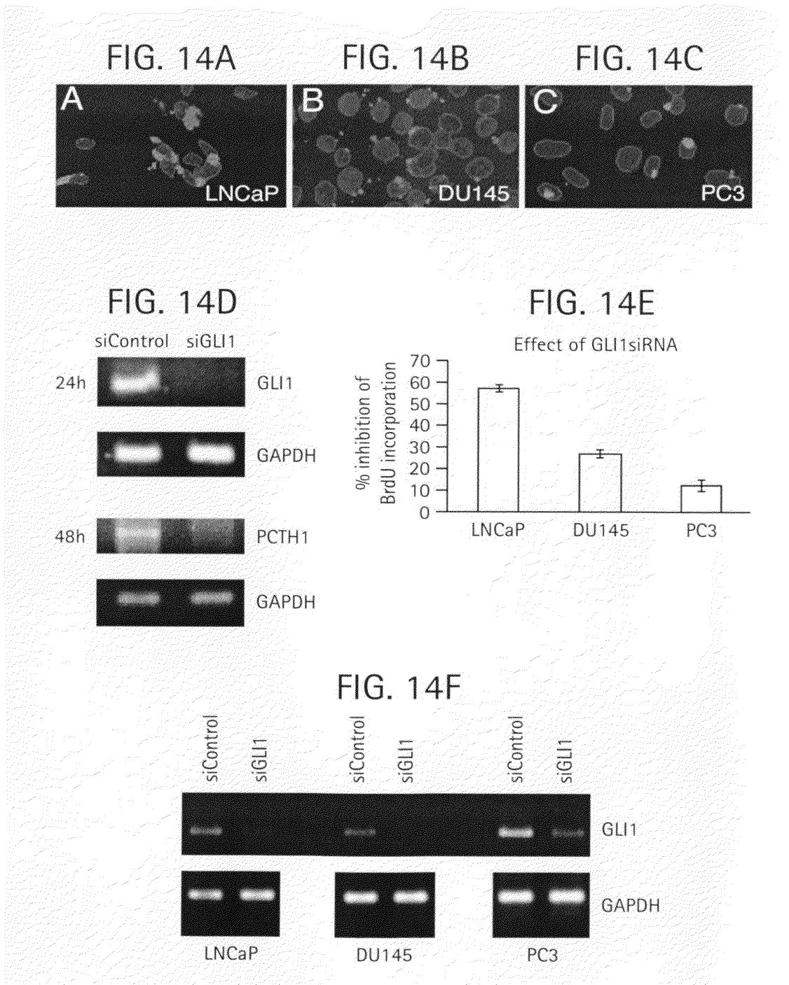

The GLI zinc-finger transcription factors have been suggested to be essential for the mediation of HH signals (Ingham, P. & McMahon, A. (2001) Genes Dev. 15, 3059-87; Ruiz i Altaba, A., Sanchez, P. & Dahmane, N. (2002) Nat. Rev. Cancer 2, 361-372 (2002); Ruiz i Altaba, A., Stecca, B. & Sanchez, P. (2004) Cancer Lett. 204, 145-157). However, Gli1 is apparently redundant in mouse development and tumorigenesis (Park H. L., Bai C., Platt K. A., Matise M. P., Beeghly A., Hui C. C., Nakashima M., Joyner A. L. (2000). Development 127, 1593-1 605; Weiner, H. L. R. Bakst, M. S. Hurlbert, J. Ruggiero, E. Ahn, W. S. Lee, D. Stephen, D. Zagzag, A. L. Joyner, D. H. (2002) Cancer Res. 62, 6385-6389), and there is to date no data on the requirement for GLI1 in human cells. Here the inventors tested the function of GLI1, the only GLI gene consistently expressed in all primary tumors and cell lines, by RNA interference to knockdown its function with a specific 21 nt-long small RNA. (This siRNA inhibits the effect of SHH on multipotent C3H10T1/2 cells; P. S, and A. R. A., unpublished). Lipofection of primary cultures resulted in a negligible number of transfected cells, making it impractical to use siRNAs in such cultures. In contrast, lipofection of FITC-siRNA proved efficient (˜50%-˜80%) in the LNCaP, PC3 and DU145 cell lines (FIG. 14A-C). It is important to note that since transfection efficiencies are under 100%, the results of cell pool assays necessarily underestimate the effects of RNA interference. Transfection of a control siRNA at the same concentration served as control in all tests.

The specificity of the GLI1 siRNA was further tested in LNCaP cells. Reduction of GUI mRNA levels by the GLI1 siRNA was detected as early as 3 h after transfection and at 24 h but not at 48 h (FIG. 14D, F and not shown), suggesting upregulation of GLI1 following its inhibition possibly due to the action of a rapid positive feedback loop (Dahmane, N., Sanchez, P., Gitton, Y., Palma, V., Sun, T., Beyna, M., Weiner, H. & Ruiz i Altaba, A. (2001). Development 128, 5201-5212; Regl, G., Neill, G. W., Eichberger, T., Kasper, M., Ikram, M. S., Koller, J., Hintner, H., Quuinn, A. G., Frischauf, A. M., Aberger, F. (2002). Oncogene. 21, 5529-39; Buttitta, L., Mo, R., Hui, C. C., Fan, C. M. (2003) Development. 130, 6233-43). GLI1 siRNA also robustly repressed PTCH1, a result most clearly seen at 48 h, but not the housekeeping gene GAPDH (FIG. 14D and not shown). Because PTCH1 is a SHH target (Goodrich, L. V., Johnson, R. L., Milenkovic, L., McMahon, J. A. & Scott, M. P. (1996) Genes Dev. 10, 301-12), and in particular of GLI1 (Buttitta, L., Mo, R., Hui, C. C., Fan, C. M. (2003) Development. 130, 6233-43), this result indicates that interference with GLI1 function by RNAi is selective and effective in prostate cancer cells. GLI1 siRNA also decreased GLI1 mRNA levels in DU145 and PC3 cells after 8 h (FIG. 14F).

Inhibition of GLI1 by RNAi led to a variable reduction in BrdU incorporation in all three cell lines, with strongest effects (˜60%) in LNCaP cells (FIG. 14E). These cells are thus very sensitive to inhibition by cyclopamine and GLI1 interference suggesting the presence of a fully active canonical pathway activated at the level of SMOH or upstream but downstream of SHH, as treatment with the blocking anti-SHH Ab had no effect. DU145 cells are not sensitive to cyclopamine but are sensitive to GLI1 interference, suggesting activation downstream of SMOH and upstream or at the level of GLI1 function. In contrast, PC3 cells are sensitive to cyclopamine and less so to GLI1 interference, perhaps suggesting compensation by the other GLI proteins since PC3 cells express GLI2 (and this GLI gene mediates SHH signals (Roessler, E., Du, Y. Z., Mullor, J. L., Casas, E., Allen, W. P., Gillessen-Kaesbach, G., Roeder, E. R., Ming, J. E., Ruiz i Altaba, A. & Muenke, M. (2003) Proc. Natl. Acad. Sci. USA, 100, 13424-9) and can behave like Gli1 in mice (Bai, C. B. & Joyner, A. L. (2001) Development 128, 5161-72) or the presence of alternate pathways for tumor cell proliferation. The inventors note, however, that lipofection efficiencies in PC3 cells (FIG. 14C) are the lowest (−50%) of the three cells tested, indicating that the real effects of GLI1 interference may be higher. Taken together, our results are the first to show the requirement of GLI1 in human tumors.

Prostate carcinomas derive from the epithelial component of the prostate gland. Tumor development occurs with progression through an in-situ lesion to become an invasive tumor that spreads within the prostate and subsequently develops the potential to metastasize to distant sites, most often the lymph nodes and bone. The three PC cell lines that the inventors have used in this study derive from metastatic lesions while primary tumor samples derive from primary moderately differentiated invasive tumors. Albeit to varying degrees, all are sensitive to inhibition of the SHH-GLI pathway at different levels. The consistent expression of GLI1 in tumor cell lines and in primary tumors together with the effects of RNA interference indicates that it plays a primary and general role in PC growth.

Here the inventors demonstrate the dependence of prostate cancer cell proliferation on SHH-GLI pathway activity. The data suggest activation of the pathway at different levels in primary prostate tumors and cell lines derived from metastatic lesions. These findings, together with the involvement of this pathway in normal prostate development and growth (Podlaseck, C. A., Barnett, H. Y., Wu, X. R., Laciak, R., Shapiro, E. & Bushman, W. (1999) Dev. Biol. 209, 28-39; Barnett, D. H., Huang, H. Y., Wu, X. R., Laciak, R., Shapiro, E. & Bushman, W. (2002) J. Urol. 168, 2206-2210; Lamm, M. L., Catbagan, W. S., Laciak, R. J., Barnett, D. H., Hebner, C. M., Gaffield, W., Walterhouse, D., Iannaccone, P. & Bushman, W. (2002) Dev. Biol. 249, 349-366; Wang, B. E., Shou, J., Ross, S., Kowppen, H., De Sauvage, F. J., Gao, W. Q. (2003) J. Biol. Chem. 278, 18506-13; Freestone, S. H., Marker, P., Grace, O. C., Tomlinson, D. C., Cunha, G. R., Hamden, P. & Thomson, A. A. (2003) Dev. Biol. 264, 352-62; Berman, D. M., Desai, N., Wang, X., Karhadkar, S. S., Reynon, M., Abate-Shen, C., Beachy, P. A. & Shen, M. M. (2004) Dev. Biol. 267, 387-98), indicate that the normal patterning role of SHH-GLI signaling is deregulated in cancer. This idea is consistent with the proposed events in other tissues, including brain, lung, stomach, muscle, pancreas, and skin, in which the SHH-GLI pathway regulates patterned growth and when deregulated can give rise to SHH-GLI-dependent tumors. Thus, there is a surprising and unexpected parallel in the requirement of SHH-GLI signaling of prostate tumors with those in organs of very different origin, function and location.

The deduction that prostate tumors display activation at different levels is consistent with findings in brain (Dahmane, N., Sanchez, P., Gitton, Y., Palma, V., Sun, T., Beyna, M., Weiner, H. & Ruiz i Altaba, A. (2001). Development 128, 5201-5212; PS and ARA, unpublished data) and pancreatic (Nelson, W. G., De Marzo, A. M. & Isaacs, W. B. (2003) N. Engl. J. Med. 349, 366-381) tumors even though the entire set of activating events or mutations have not been described in any case. Indeed, our data indicate that the regulation of the SHH-GLI pathway in the normal prostatic epithelium is altered away from homeostasis in the tumors by epigenetic events or mutations in components such as PTCH1, SMOH or SUFUH, similar to those already found in other tumors (Pietsch, T. A. Waha, A. Koch, J. Kraus, S. Albrecht, J. Tonn, N. Sorensen, F. Berthold, B. Henk, N. Schmandt, et al. (1997) Cancer Res 57 2085-2088; Raffel, R. B. Jenkins, L. Frederick, D. Hebrink, B. Alderete, D. W. Fults, C. D. James, (1997) Cancer Res. 57 842-845; Wolter, M. J. Reifenberger, C. Sommer, T. Ruzicka, G. Reifenberger, (1997) Cancer Res. 57 2581-2585; Reifenberger, J. M. Wolter, R. G. Weber, M. Megahed, T. Ruzicka, P. Lichter, G. Reifenberger, (1998) Cancer Res. 58 1798-1803; Dong, J. M. R. Gailani, S. L. Pomeroy, D. Reardon, A. E. Bale, (2000) Hum. Mutat. 16 89-90; Zurawel, R. H. C. Allen, S. Chiappa, W. Cato, J. Biegel, P. Cogen, F. de Sauvage, C. Raffel, (2000) Genes Chromosomes Cancer 27 44-51; Taylor, M. D. L. Liu, C. Raffel, C. C. Hui, T. G. Mainprize, X. Zhang, R. Agatep, S. Chiappa, L. Gao, A. Lowrance, et al. (2002) Nat. Genet. 31 306-310). However, the finding that the pathway is active as assessed by the expression of GLI1 and PTCH1 (as in the case of basal cell carcinomas (Dahmane, N., Lee, J., Robins, P., Heller, P. & Ruiz i Altaba, A. (1997) Nature 389, 876-81), medulloblastomas (Dahmane, N., Sanchez, P., Gitton, Y., Palma, V., Sun, T., Beyna, M., Weiner, H. & Ruiz i Altaba, A. (2001). Development 128, 5201-5212) and gliomas (Dahmane, N., Sanchez, P., Gitton, Y., Palma, V., Sun, T., Beyna, M., Weiner, H. & Ruiz i Altaba, A. (2001). Development 128, 5201-5212. Dahmane, N., Sanchez, P., Gitton, Y., Palma, V., Sun, T., Beyna, M., Weiner, H. & Ruiz i Altaba, A. (2001). Development 128, 5201-5212) allows us to bypass the identification of the likely myriad of activating events to discern that tumor cells harbor an active pathway. Indeed, the finding that SHH expression levels are not correlated with Gleason score but that all prostate tumor samples tested require continued pathway activity for proliferation allows us to propose that this pathway is a critical and essential component of prostate cancer.

Specifically, the inventors show the requirement for SHH, SMOH and/or GLI1 for the proliferation of prostate cancer cells. The fact that all primary tumors tested are sensitive to cyclopamine indicates that SMOH, or upstream elements from it, are common targets leading to the activation of downstream mediators. Several primary cultures are also sensitive to inhibition by blocking anti-SHH Ab, suggesting that, like in stomach tumors (Berman, D. M., Karhadkar, S. S., Maitra, A., Montes De Oca, R., Gerstenblith, M. R., Briggs, K., Parker, A. R., Shimada, Y., Eshleman, J. R., Watkins, D. N. & Beachy, P. A. (2003) Nature. 425, 846-51), autocrine signaling is a frequent cause of pathway activation in prostate cancer. The consistent expression of GLI1 in tumor cell lines and in primary tumors together with the effects of RNA interference indicate that this GLI gene plays a central and general role in prostate tumor cell proliferation. This is the first demonstration of the requirement of GLI1 in human tumorigenesis. In contrast, GLI2 and GLI3 do not appear to be consistently expressed in prostate cancer cells. When expressed they could have complementary or compensatory roles in some cases although their roles remains to be determined.

Prostate cancer is thought to develop from a lesion in the epithelial layer to become an invasive tumor that spreads within the prostate and subsequently acquires the potential to metastasize to distant sites, most often the lymph nodes and bone (Abate-Shen, C. & Shen, M. (2000). Genes and Dev. 14, 2410-2434). Inhibition of testosterone-dependent tumor growth is the common treatment for advanced disease, but subsequent hormone-independent cell proliferation and metastasis often leads to patient death (Martel, C. L., Gumerlock, P. H., Meyers, F. J. & Lara, P. N. (2003) Cancer Treat Rev. 29, 171-187). The data on the behavior of the three prostate cancer cell lines derived from metastatic lesions suggest that such tumors could harbor additional changes that may make them ligand-independent, albeit still being SHH-GLI pathway dependent, and explain their differential behavior in comparison with the primary cultures. Perhaps the gain of intracellular, cell-autonomous activation of the SHH-GLI pathway represents an advantage for metastatic cells, allowing efficient proliferation far from the prostatic epithelium, where SHH is continuously produced.

The high inhibition of proliferation by SHH-GLI pathway blockade of the presumed androgen-sensitive primary tumors used in this study, which derive from patients that did not receive hormone treatments, and of the androgen-sensitive LNCaP cell line, might be related to the proposed requirement of Shh signaling for normal androgen function, as defects derived from loss of Shh signaling in mice can be rescued by exogenous androgens (Berman, D. M., Desai, N., Wang, X., Karhadkar, S. S., Reynon, M., Abate-Shen, C., Beachy, P. A. & Shen, M. M. (2004) Dev. Biol. 267, 387-98). Prostate cancer could therefore initiate through inappropriate maintenance or enhanced activity of SHH-GLI signaling and more aggressive (androgen insensitive) states may require additional alterations. Nevertheless, the inhibition of the androgen-insensitive DU145 cell line by RNAi suggests that even highly aggressive tumors may be sensitive, albeit to different degrees, to GLI1 inhibition.

Prostate stem cells may play a critical role in the epithelial development and homeostasis. Since cancer may be a disease of stem cell lineages (Ruiz i Altaba, A., Sanchez, P. & Dahmane, N. (2002) Nat. Rev. Cancer 2, 361-372 (2002); Pasca di Magliano M, Hebrok M. (2003) Nat. Rev. Cancer. 3, 903-11; Ruiz i Altaba, A., Stecca, B. & Sanchez, P. (2004) Cancer Lett. 204, 145-157; Reya, T., Morrison, S. J., Clarke, M. F. & Weissman, I. L. (2001) Nature, 414, 105-111) and SHH-GLI signaling controls the behavior of precursors and of cells with stem cell properties in the mammalian brain (Palma, V. & Ruiz i Altaba, A. (2004) Development. 131, 337-345; Lai, K., Kaspar, B. K., Gage, F. H. & Schaffer, D. V. (2003) Nat. Neurosci. 6, 21-27; Machold, R., Hayashi, S., Rutlin, M., Muzumdar, M. D., Nery, S., Corbin, J. G., Gritli-Linde, A., Dellovade, T., Porter, J. A., Rubin, L. L. et al. (2003) Neuron 39, 937-50) and in other tissues and species (Zhang, Y. & Kalderon, D. (2001) Nature. 410, 599-604; Park, Y., Rangel, C., Reynolds, M. M., Caldwell, M. C., Johns, M., Nayak, M., Welsh, C. J., McDermott, S. & Datta, S. (2003) Dev Biol. 253, 247-57), prostate cancer might derive from inappropriate expansion of prostatic epithelial stem cell lineages due to abnormal SHH-GLI function.

Finally, the data presented herein suggest that SHH and GLI1 may not only be useful markers for prostate cancer but also good targets for anti-cancer therapies, with emphasis on GLI function as the last and essential step of the pathway, the inhibition of which will likely block signaling by upstream events at any level. SHH-GLI pathway blocking agents should thus provide attractive therapeutic strategies to combat prostate cancer of any grade.

TABLE 1
Gene expression in human prostate cancer

| | SHH | | PTCH1 | | GLI1 | | GLI2 | | GLI3 | |
|---|---|---|---|---|---|---|---|---|---|---|
| Patient | Fold inc. | range | Fold inc. | range | Fold inc. | range | Fold inc. | range | Fold inc. | range |
| 829 | 0 | 0-0.01 | 1.5 | 1.1-2.1 | 26.1 | 20.8-32.7 | 0.02 | 0.02-0.02 | 72 | 53-99 |
| 887 | 0.2 | 0.05-0.9 | 8.5 | 7.6-9.5 | 0.09 | 0.07-0.13 | 0.43 | 0.37-0.51 | 1.1 | 0.8-1.6 |
| 921 | 2.9 | 1.3-6.3 | 50 | 30-84 | 2 | 1.1-3.4 | 3.8 | 2.4-6.1 | 12.5 | 7.7-20.4 |
| 945 | 9.8 | 6.2-15.7 | 7.8 | 5.7-10.7 | 22.7 | 21.6-23.9 | 0.7 | 0.5-1.0 | 2.2 | 2.1-2.4 |
| 1854 | 4.7 | 1.8-11.7 | 213 | 164-278 | 5.1 | 3.8-6.9 | 19.5 | 10.9-35.1 | 5.7 | 4.4-7.5 |
| 1866 | 4.6 | 4.1-5.2 | 3.4 | 3.1-3.7 | 299 | 260-342 | 0.03 | 0.02-0.03 | 0.18 | 0.15-0.2 |

Fold increase in gene expression in tumors versus matched normal tissue determined by real time RT-PCR analyses as calculated by the delta delta CT method.
Range indicates +/− one standard deviation.
Gene expression levels were normalized to beta actin. Increases of 2 fold or more are shown in bold.

TABLE 2
Correlation of elevated SHH expression with tumorigenesis
and clinical features of prostate cancer

| | | SHH | SHH | Chi squared |
|---|---|---|---|---|
| | | expression | expression | or Fisher's |
| | | low | high | exact test |
| Histology | Tumor | 141 | 70 | p < 0.00005 |
| | Normal | 126 | 1 | |
| | HGPIN | 13 | 1 | |
| | Normal | 126 | 1 | p = 0.0563 |
| Clinical stage | cT2 | 16 | 6 | |
| | cT3/4 | 2 | 1 | N.S. |
| Tumor grade | Gleason 6 | 30 | 1 | |
| | Gleason 7, 8, 9 | 57 | 7 | N.S. |
| Pathologic | pT1-pT2 | 50 | 4 | |
| stage | pT3 | 37 | 4 | N.S. |
| Nodal status | pN0 | 27 | 12 | |
| | pN1 | 1 | 0 | N.S. |
| Outcomes | PSA Recurrence | 8 | 1 | |
| | No PSA | 22 | 12 | N.S. |
| | recurrence | | | |
| Vital status | Alive | 42 | 18 | |
| | Dead | 4 | 3 | N.S |

| | | Mean % Ki-67⁺ | Two-tailed |
|---|---|---|---|
| Ki-67 expression | sample number | nuclei | ANOVA |
| SHH high | 69 | 7.6 | |
| SHH low | 275 | 5.1 | p = 0.0141 |

Table 2 above shows the correlation of the expression of SHH protein in prostate normal and tumor microarrays with clinical and pathological criteria. Significance was only found between SHH expression and tumorigenesis and between SHH expression and higher proliferative levels as measured by Ki-67 staining. Tumor grade is presented as Gleason score. Pathologic staging uses the American Joint Commission on Cancer (AJCC) 2002 tumor staging criteria. HGPIN=high grade prostatic intraepithelial neoplasia.

What is claimed is:

1. A method for inhibiting the synthesis and/or expression and/or activity of a GLI protein in a cell in vitro or in vivo comprising, contacting said cell with or introducing into said cell an inhibitor or antagonist of GLI synthesis and/or expression and/or activity, said inhibitor or antagonist selected from the group consisting of a small molecule inhibitor or antagonist of GLI synthesis and/or expression and/or activity, an antisense molecule or a siRNA molecule that is complementary to at least a part of the sequence of a nucleic acid encoding the GLI protein, antibodies to GLI, and agents that exhibit mimicry to GLI or antagonism to it or control over its production, said contacting or introducing resulting in inhibition of synthesis and/or expression and/or activity of the GLI protein in said cell.

2. The method of claim 1, wherein said introducing further results in inhibition of cellular proliferation and/or inhibition of tumorigenesis.

3. The method of claim 1, wherein said cell is a tumor cell.

4. The method of claim 3, wherein said tumor cell is selected from the group consisting of gliomas, medulloblastomas, primitive neuroectodermal tumors (PNETS), basal cell carcinoma, small cell lung cancers, large cell lung cancers, tumors of the gastrointestinal tract, rhabdomyosarcomas, soft tissue sarcomas, pancreatic tumors, bladder tumors and prostate tumors.

5. The method of claim 1, wherein said GLI protein is selected from the group consisting of GLI1, GLI2 and GLI3.

6. The method of claim 1, wherein said antagonist comprises an antisense compound comprising an oligonucleotide of about 10 to 50 nucleobases in length targeted to a nucleic acid encoding a GLI protein, wherein said antisense compound inhibits the expression and/or activity of said GLI protein.

7. The method of claim 6, wherein said antisense compound has the sequence as set forth in SEQ ID NO: 33.

8. The method of claim 1, wherein said antagonist comprises a siRNA molecule, said siRNA molecule comprising a double stranded structure having a nucleotide sequence which is substantially identical to or complementary to at least a part of the gli gene in an amount sufficient to inhibit the synthesis and/or expression and/or activity of a gli gene.

9. The method of claim 8, wherein said gli gene is selected from the group consisting of gli1, gli2 and gli3.

10. The method of claim 8, wherein said siRNA hybridizes under stringent conditions to the gli gene.

11. The method of claim 8, wherein the siRNA molecule is about 20 nucleotides in length.

12. The method of claim 11, wherein the siRNA molecule is selected from the group consisting of the nucleic acid sequences set forth in SEQ ID NO: 29, SEQ ID NO: 31 and SEQ ID NO: 32.

13. The method of claim 1, wherein said antagonist is a small molecule inhibitor of GLI expression and/or synthesis and/or activity.

14. The method of claim 13, wherein said small molecule inhibitor is a plant alkaloid or its analogs or derivatives thereof.

15. The method of claim 14, wherein said plant alkaloid is cyclopamine or jervine or their analogs or derivatives thereof.

16. The method of claim 1, wherein said antagonist is an antibody specific for at least one of the GLI proteins.

17. The method of claim 16, wherein said antibody is a polyclonal antibody.

18. The antibody of claim 16, wherein said antibody is a monoclonal antibody.

19. An immortal cell line that produces a monoclonal antibody according to claim 18.

20. The antibody of either of claim 17 or 18, wherein said antibody is a blocking antibody and is effective as a therapeutic agent to prevent the proliferation of tumor cells in vivo.

21. The antibody of either one of claim 17 or 18, wherein said antibody is coupled to an anti-tumor drug or radioisotope for use in treating tumor cells in situ.

22. The antibody of either of claim 17 or 18, wherein said antibody is effective as a diagnostic agent to aid in identification of tumor cells in situ or in vivo.

23. The antibody of claim 22, wherein said antibody is coupled to an imaging reagent and is used for imaging of tumor cells in situ or in vivo.

24. A method of preventing and/or treating cellular debilitations, derangements and/or dysfunctions and/or hyperplastic/hyperproliferative and/or cancerous disease states in a mammal characterized by the presence and/or expression of a GLI gene or gene product, comprising administering to a mammal a therapeutically effective amount of an agent that inhibits or antagonizes the synthesis and/or expression and/or activity of a GLI molecule.

25. The method of claim 24, wherein said inhibitors/antagonists are selected from the group consisting of small molecule antagonists of GLI expression and activity, antisense compounds, siRNA nucleic acids, antibodies to GLI, and agents that exhibit mimicry to GLI or antagonism to it or control over its production.

26. The method of claim 24, wherein said GLI molecule is selected from the group consisting of GLI1, GLI2 and GLI3.

27. The method of claim 24, wherein said hyperproliferative and/or cancerous disease states are selected from the group consisting of gliomas, medulloblastomas, primitive neuroectodermal tumors (PNETS), basal cell carcinoma, small cell lung cancers, large cell lung cancers, tumors of the gastrointestinal tract, rhabdomyosarcomas, soft tissue sarcomas, pancreatic tumors, bladder tumors and prostate tumors.

28. The method of claim 25, wherein said antagonist comprises an antisense compound comprising an oligonucleotide of about 10 to 50 nucleobases in length targeted to a nucleic acid encoding a GLI protein, wherein said antisense compound inhibits the expression and/or activity of said GLI protein.

29. The method of claim 28, wherein said antisense compound has the sequence as set forth in SEQ ID NO: 33.

30. The method of either one of claim 28 or 29, wherein said antisense compound, upon administering to said mammal, results in the inhibition of expression and/or activity of said GLI protein in the cells of said mammal.

31. The method of claim 30, wherein the delivery of said antisense compound to said mammal further results in inhibition of cellular proliferation in said mammal.

32. The method of claim 25, wherein said antagonist comprises a siRNA molecule, said siRNA molecule comprising a double stranded structure having a nucleotide sequence which is substantially identical to at least a part of the gli gene in an amount sufficient to inhibit the synthesis and/or expression and/or activity of a gli gene.

33. The method of claim 32, wherein said gli gene is selected from the group consisting of gli1, gli2 and gli3.

34. The method of claim 32, wherein said siRNA hybridizes under stringent conditions to the gli gene.

35. The method of claim 32, wherein the siRNA molecule is about 20 nucleotides in length.

36. The method of claim 35, wherein the siRNA molecule is selected from the group consisting of the nucleic acid sequences set forth in SEQ ID NO: 29, SEQ ID NO: 31 and SEQ ID NO: 32.

37. The method of claim 25, wherein said antagonist is a small molecule inhibitor of GLI expression and/or synthesis and/or activity.

38. The method of claim 37, wherein said small molecule inhibitor is a plant alkaloid or its analogs or derivatives thereof.

39. The method of claim 38, wherein said plant alkaloid is cyclopamine, jervine or their analogs or derivatives thereof.

40. The method of claim 25, wherein said antagonist is an antibody specific for at least one of the GLI proteins.

41. The method of claim 40, wherein said antibody is a polyclonal antibody.

42. The antibody of claim 40, wherein said antibody is a monoclonal antibody.

43. An immortal cell line that produces a monoclonal antibody according to claim 42.

44. The antibody of either of claim 41 or 42, wherein said antibody is a blocking antibody and is effective as a therapeutic agent to prevent the proliferation of tumor cells in vivo.

45. The antibody of either one of claim 41 or 42, wherein said antibody is coupled to an anti-tumor drug or radioisotope for use in treating tumor cells in situ.

46. A method of inducing a tumor cell to undergo apoptosis comprising administering an inhibitor or antagonist to the SHH/GLI signaling pathway in the cell.

47. The method of claim 46, wherein said inhibitor or antagonist is selected from the group consisting of a small molecule inhibitor or antagonist of GLI synthesis and/or expression and/or activity, an antisense molecule or a siRNA molecule that is complementary to at least a part of the sequence of a nucleic acid encoding the GLI protein, antibodies to GLI, and agents that exhibit mimicry to GLI or antagonism to it or control over its.

48. The method of claim 47, wherein said antisense molecule comprises the sequence as set forth in SEQ ID NO: 33.

49. The method of claim 47, wherein said siRNA molecule is selected from the group consisting of the nucleic acid sequences of SEQ ID NO: 29, SEQ ID NO: 31 and SEQ ID NO: 32.

50. The method of claim 47, wherein the small molecule inhibitor of GLI synthesis and/or expression and/or activity is cyclopamine, jervine or analogs or derivatives thereof.

51. A method of overcoming resistance to chemotherapeutic agents in tumor cells, comprising administering an antagonist or inhibitor to the SHH/GLI signaling pathway to the cell, wherein said administering results in increased sensitivity of the tumor cell to said chemotherapeutic agent and results in subsequent tumor cell death and prevention from metastasis, and wherein said antagonist or inhibitor is selected from the group consisting of a small molecule inhibitor or antagonist of GLI synthesis and/or expression and/or activity, an antisense molecule or a siRNA molecule that is complementary to at least a part of the sequence of a nucleic acid encoding the GLI protein, antibodies to GLI, and agents that exhibit mimicry to GLI or antagonism to it or control over its.

52. The method of claim 51, wherein said antisense molecule comprises a nucleic acid sequence that is complementary to the nucleic acid that encodes at least one GLI protein.

53. The method of claim 52, wherein said antisense molecule comprises the nucleic acid sequence as set forth in SEQ ID NO: 33.

54. The method of claim 51, wherein said siRNA molecule is selected from the group consisting of the nucleic acid sequences of SEQ ID NO: 29, SEQ ID NO: 31 and SEQ ID NO: 32.

55. The method of claim 51, wherein the small molecule inhibitor of GLI synthesis and/or expression and/or activity is cyclopamine, jervine or analogs or derivatives thereof.

54. A method for inhibiting and/or attenuating the expression of a gli gene in a mammalian cell, said method comprising:
a) introducing into the cell an RNA having a nucleotide sequence which is substantially identical to or complementary to at least a part of the gli gene in an amount sufficient to inhibit and/or attenuate expression of a gli gene;
b) verifying inhibition and/or attenuation of expression of the gli gene; and
wherein the mammalian cell is a tumor cell or a cell obtained from tissue exhibiting hyperplasia that is characterized by the expression of at least one gli gene or gene product.

57. The method of claim 56, wherein said gli gene is selected from the group consisting of gli1, gli2 and gli3.

58. The method of claim 56, wherein said RNA is a single stranded RNA (SS-RNA) or a double stranded RNA (DS-RNA) molecule.

59. The method of claim 58, wherein said SS-RNA molecule is an antisense RNA molecule, which when administered to a mammal having a tumor cell characterized by the presence of said gli gene, results in inhibition of tumor cell proliferation.

60. The method of claim 58, wherein said DS-RNA is a siRNA molecule, which when administered to a mammal having a tumor cell characterized by the presence of said gli gene, results in inhibition of tumor cell proliferation.

61. The method of claim 60, wherein said siRNA hybridizes under stringent conditions to the gli gene.

62. The method of claim 60, wherein the siRNA molecule is about 20 nucleotides in length.

63. The method of claim 62, wherein the siRNA molecule is selected from the group consisting of the nucleic acid sequences set forth in SEQ ID NO: 29, SEQ ID NO: 31 and SEQ ID NO: 32.

64. A double stranded siRNA for inhibiting expression of a gli gene, comprising a first nucleotide sequence that hybridizes under stringent conditions to a nucleotide sequence of a gli gene and a second nucleotide sequence that is complementary to said first nucleotide sequence.

65. The siRNA of claim 64, wherein said first nucleotide sequence of said siRNA is about 20 nucleotides long.

66. The siRNA of claim 64, wherein said siRNA is selected from the group consisting of the nucleic acid sequences as set forth in SEQ ID NO: 29, SEQ ID NO: 31 and SEQ ID NO: 32.

67. The siRNA of any one of claims 64 through 66, wherein said siRNA, upon delivery to a cell containing the gli gene for which said siRNA molecules are specific, inhibits the proliferation of said cell.

68. The double stranded siRNA of claim 64, wherein the double stranded RNA is a hairpin comprising a first nucleotide sequence that hybridizes under stringent conditions to a nucleotide sequence of a gli gene, and a second nucleotide sequence which is a complementary inverted repeat of said first nucleotide sequence and hybridizes to said first nucleotide sequence to form a hairpin structure.

69. A pharmaceutical composition comprising at least one inhibitor or antagonist of GLI synthesis and/or expression and/or activity and a pharmaceutically acceptable carrier.

70. The pharmaceutical composition of claim 69, wherein said inhibitor or antagonist is selected from the group consisting of a small molecule inhibitor or antagonist of GLI synthesis and/or expression and/or activity, an antisense molecule or a siRNA molecule that is complementary to at least a part of the sequence of a nucleic acid encoding the GLI protein, antibodies to GLI, and agents that exhibit mimicry to GLI or antagonism to it or control over its production, said contacting or introducing resulting in inhibition of synthesis and/or expression and/or activity of the GLI protein in said cell.

71. The pharmaceutical composition of claim 70, wherein said antisense molecule comprises the nucleic acid sequence as set forth in SEQ ID NO: 33.

72. The pharmaceutical composition of claim 70, wherein said siRNA molecule comprises the nucleic acid sequence as set forth in SEQ ID NOs: 29, 31 and 32.

73. The pharmaceutical composition of claim 70, wherein said small molecule inhibitor is cyclopamine, jervine or analogs or derivatives thereof.

74. A method of identifying a subject that is likely to be suffering from a hyperproliferative condition or a cancerous condition comprising:
determining the level of at least one GLI protein or a nucleic acid encoding at least one GLI protein in a tissue specimen or in a specimen of bodily fluid from said subject; wherein when the level of GLI protein or a nucleic acid encoding said GLI protein determined is significantly elevated in cells obtained from said tissue or bodily fluid compared to a predetermined range of normal values established from screening individuals known to be free of a hyperproliferative or cancerous condition, the animal subject is identified as being likely to have a hyperproliferative condition or a cancerous condition.

75. The method of claim 74 wherein the determining is performed in situ or in vitro.

76. The method of claim 74, wherein the determining is performed by a method that uses a probe selected from the group consisting of an antibody specific for at least one GLI protein; a set of primers specific for an mRNA encoding said GLI protein; and a nucleotide probe specific for an mRNA encoding said GLI protein.

77. The method of claim 76, wherein said bodily fluid is selected from the group consisting of whole blood, serum, plasma, urine and cerebrospinal fluid.

78. A method of predicting resistance of a tumor cell to chemotherapeutic agents comprising measuring the level of expression of GLI in said tumor cell, whereby an increased level of expression of GLI in said tumor cell is predictive of resistance of said tumor cell to chemotherapeutic agents.

79. The method of claim 78, wherein said tumor cells are selected from the group consisting of gliomas, medulloblastomas, primitive neuroectodermal tumors (PNETS), basal cell carcinoma, small cell lung cancers, large cell lung cancers, tumors of the gastrointestinal tract, rhabdomyosarcomas, soft tissue sarcomas, pancreatic tumors and prostate tumors.

80. The method of either of claim 46 or 47, wherein said antagonist comprises agents that increase negative, or decrease positive acting elements affecting SHH-GLI signaling, said agents selected from the group consisting of small molecules that alter PKA kinase activity, small molecules that alter GSK3 kinase activity, small molecules that alter CK1 kinase activity, and small molecules or siRNAs/antisense RNAs that alter DYRK1 kinase activity.

81. A method of screening for apoptotic resistant tumor cells, comprising measuring the level of expression of GLI in said tumor cells, wherein enhanced expression of GLI correlates with apoptosis resistance in said tumor cells.

82. The method of claim 81, wherein said tumor cells are selected from the group consisting of gliomas, medulloblastomas, primitive neuroectodermal tumors (PNETS), basal cell carcinoma, small cell lung cancers, large cell lung cancers, tumors of the gastrointestinal tract, rhabdomyosarcomas, soft tissue sarcomas, pancreatic tumors, bladder tumors and prostate tumors.


SEQUENCE LISTING (35 sequences)

SEQ ID NO: 1 - DNA, 1575 bp, Homo sapiens
gcgaggcagccagcgagggagagagcgagcgggcgagccggagcgaggaagggaaagcgcaagagagagcgcacacgcacacacccgccgcgcgcactcgcgcacggacccgcacggggacagctcggaagtcatcagttccatgggcgagatgctgctgctggcgagatgtctgctgctagtcctcgtctcctcgctgctggtatgctcgggactggcgtgcggaccgggcagggggttcgggaagaggaggcaccccaaaaagctgacccctttagcctacaagcagtttatccccaatgtggccgagaagaccctaggcgccagcggaaggtatgaagggaagatctccagaaactccgagcgatttaaggaactcacccccaattacaaccccgacatcatatttaaggatgaagaaaacaccggagcggacaggctgatgactcagaggtgtaaggacaagttgaacgctttggccatctcggtgatgaaccagtggccaggagtgaaactgcgggtgaccgagggctgggacgaagatggccaccactcagaggagtctctgcactacgagggccgcgcagtggacatcaccacgtctgaccgcgaccgcagcaagtacggcatgctggcccgcctggcggtggaggccggcttcgactgggtgtactacgagtccaaggcacatatccactgctcggtgaaagcagagaactcggtggcggccaaatcgggaggctgcttcccgggctcggccacggtgcacctggagcagggcggcaccaagctggtgaaggacctgagccccggggaccgcgtgctggcggcggacgaccagggccggctgctctacagcgacttcctcactttcctggaccgcgacgacggcgccaagaaggtcttctacgtgatcgagacgcgggagccgcgcgagcgcctgctgctcaccgccgcgcacctgctctttgtggcgccgcacaacgactcggccaccggggagcccgaggcgtcctcgggctcggggccgccttccgggggcgcactggggcctcgggcgctgttcgccagccgcgtgcgcccgggccagcgcgtgtacgtggtggccgagcgtgacggggaccgccggctcctgcccgccgctgtgcacagcgtgaccctaagcgaggaggccgcgggcgcctacgcgccgctcacggcccagggcaccattctcatcaaccgggtgctggcctcgtgctacgcggtcatcgaggagcacagctgggcgcaccgggccttcgcgcccttccgcctggcgcacgcgctcctggctgcactggcgcccgcgcgcacggaccgcggcggggacagcggcggcggggaccgcgggggcggcggcggcagagtagccctaaccgctccaggtgctgccgacgctccgggtgcgggggccaccgcgggcatccactggtactcgcagctgctctaccaaataggcacctggctcctggacagcgaggccctgcacccgctgggcatggcggtcaagtccagctgaagccggggggccgggggaggggcgcgggagggggc

SEQ ID NO: 2 - protein, 462 aa, Homo sapiens
MLLLARCLLLVLVSSLLVCSGLACGPGRGFGKRRHPKKLTPLAYKQFIPNVAEKTLGASGRYEGKISRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKAHIHCSVKAENSVAAKSGGCFPGSATVHLEQGGTKLVKDLSPGDRVLAADDQGRLLYSDFLTFLDRDDGAKKVFYVIETREPRERLLLTAAHLLFVAPHNDSATGEPEASSGSGPPSGGALGPRALFASRVRPGQRVYVVAERDGDRRLLPAAVHSVTLSEEAAGAYAPLTAQGTILINRVLASCYAVIEEHSWAHRAFAPFRLAHALLAALAPARTDRGGDSGGGDRGGGGGRVALTAPGAADAPGAGATAGIHWYSQLLYQIGTWLLDSEALHPLGMAVKSS

SEQ ID NO: 3 - DNA, 1314 bp, Mus musculus
atgctgctgctgctggccagatgttttctggtgatccttgcttcctcgctgctggtgtgccccgggctggcctgtgggcccggcagggggtttggaaagaggcggcaccccaaaaagctgacccctttagcctacaagcagtttattcccaacgtagccgagaagaccctaggggccagcggcagatatgaagggaagatcacaagaaactccgaacgatttaaggaactcacccccaattacaaccccgacatcatatttaaggatgaggaaaacacgggagcagaccggctgatgactcagaggtgcaaagacaagttaaatgccttggccatctctgtgatgaaccagtggcctggagtgaagctgcgagtgaccgagggctgggatgaggacggccatcattcagaggagtctctacactatgagggtcgagcagtggacatcaccacgtccgaccgggaccgcagcaagtacggcatgctggctcgcctggctgtggaagcaggtttcgactgggtctactatgaatccaaagctcacatccactgttctgtgaaagcagagaactccgtggcggccaaatccggcggctgtttcccgggatccgccaccgtgcacctggagcagggcggcaccaagctggtgaaggacttacgtcccggagaccgcgtgctggcggctgacgaccagggccggctgctgtacagcgacttcctcaccttcctggaccgcgacgaaggcgccaagaaggtcttctacgtgatcgagacgctggagccgcgcgagcgcctgctgctcaccgccgcgcacctgctcttcgtggcgccgcacaacgactcggggcccacgcccgggccaagcgcgctctttgccagccgcgtgcgccccgggcagcgcgtgtacgtggtggctgaacgcggcggggaccgccggctgctgcccgccgcggtgcacagcgtgacgctgcgagaggaggaggcgggcgcgtacgcgccgctcacggcgcacggcaccattctcatcaaccgggtgctcgcctcgtgctacgctgtcatcgaggagcacagctgggcacaccgggccttcgcgcctttccgcctggcgcacgcgctgctggccgcgctggcacccgcccgcacggacggcgggggcgggggcagcatccctgcagcgcaatctgcaacggaagcgaggggcgcggagccgactgcgggcatccactggtactcgcagctgctctaccacattggcacctggctgttggacagcgagaccatgcatcccttgggaatggcggtcaagtccagctga

SEQ ID NO: 4 - protein, 429 aa, Mus musculus
FLVILASSLLVCPGLACGPGRGFGKRRHPKKLTPLAYKQFIPNVAEKTLGASGRYEGKITRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKAHIHCSVKAENSVAAKSGGCFPGSATVHLEQGGTKLVKDLRPGDRVLAADDQGRLLYSDFLTFLDRDEGAKKVFYVIETLEPRERLLLTAAHLLFVAPHNDSGPTPGPSALFASRVRPGQRVYVVAERGGDRRLLPAAVHSVTLREEEAGAYAPLTAHGTILINRVLASCYAVIEEHSWAHRAFAPFRLAHALLAALAPARTDGGGGGSIPAAQSATEARGAEPTAGIHWYSQLLYHIGTWLLDSETMHPLGMAVKSS

SEQ ID NO: 5 - DNA, 1714 bp, Rattus norvegicus
ttaaaatcaggctctttttgtcttttaattgccgtctcgagacccaactccgatgtgttccgttaccagcgaccggcagcctgccatcgcagcccctgtctgggtggggatcggagacaagtcccctgcagcaacagcaggcaaggttatataggaagagaaagagccaggcagcgccagagggaacgaacgagccgagcgaggaagggagagccgagcgcaaggaggagcgcacacgcacacacccgcgcgtaccagctcgcgcacagaccggcgcggggacggctcgcaagtcctcaggttccgcggacgagatgctgctgctgctggccagatgttttctggtggcccttgcttcctcgctgctggtgtgccccggactggcctgtgggcccggcagggggtttggaaagaggcagcaccccaaaaagctgacccctttagcctacaagcagtttatccccaacgtagccgagaagaccctaggggccagcggccgatatgaagggaagatcacaagaaactccgaacgatttaaggaactcacccccaattacaaccccgacatcatatttaaggatgaggaaaacactggagcagaccggctgatgactcagaggtgcaaagacaagttaaatgccttggccatctccgtgatgaaccagtggcctggagtgaagcttcgagtgactgagggctgggatgaggacggccatcattcagaggagtctctacactatgagggtcgagcagtggacatcaccacgtctgacagggaccgcagcaagtatggcatgctggctcgcctggctgtggaggctggattcgactgggtctactatgaatccaaagctcgcatccactgctctgtgaaagcagagaactccgtggcggccaaatctgacggctgcttcccgggatcagccacagtgcacctggagcagggtggcaccaagttagtgaaggatctaagtcccggggaccgcgtgctggcggctgacgaccagggccggctgctgtacagcgacttcctcaccttcctggaccgcgacgaaggtgccaagaaggtcttctacgtgatcgagacgcgggagccgcgggagcgtctgctgctcactgccgcgcacctgctcttcgtggcgccgcacaacgactccgggcccactccgggaccgagcccactcttcgccagccgcgtgcgtccggggcagcgcgtgtacgtggtggctgaacgcggcggggaccgccggctgctgcccgccgcggtgcacagcgtaacgctacgagaggaggcggcgggtgcgtacgcgccgctcacggcggacggcaccattctcatcaaccgggtgctcgcctcgtgctacgcagtcatcgaggagcacagctgggcacaccgggccttcgcgcccttccgcctggcgcacgcgctgctggccgcgctggcacccgcccgcacggacggcgggggcgggggcagcatccctgccccgcaatctgtagcggaagcgaggggcgcagggccgcctgcgggcatccactggtactcgcagctgctgtaccacattggcacctggctgttggacagcgagaccctgcatcccttgggaatggcagtcaagtccagctgaagtccgacgggaccgggcagggggcgtgggggcgggcgggcgggaagcgactgccagataagcaaccgggaaagcgcacggaagga

SEQ ID NO: 6 - protein, 437 aa, Rattus norvegicus
MLLLLARCFLVALASSLLVCPGLACGPGRGFGKRQHPKKLTPLAYKQFIPNVAEKTLGASGRYEGKITRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKARIHCSVKAENSVAAKSDGCFPGSATVHLEQGGTKLVKDLSPGDRVLAADDQGRLLYSDFLTFLDRDEGAKKVFYVIETREPRERLLLTAAHLLFVAPHNDSGPTPGPSPLFASRVRPGQRVYVVAERGGDRRLLPAAVHSVTLREEAAGAYAPLTADGTILINRVLASCYAVIEEHSWAHRAFAPFRLAHALLAALAPARTDGGGGGSIPAPQSVAEARGAGPPAGIHWYSQLLYHIGTWLLDSETLHPLGMAVKSS

SEQ ID NO: 7 - DNA, 1546 bp, Xenopus laevis
cgagcagagattgcccataattactgtctcgtctctacacccccatgtgttctgtgagcggggagctgcaccctggactttctgcacctgccttgcttgggatcggtggctagaggggtcggcgaggaggcacaaggttgctggaagcagcagcgaaggagaacatcctctgagcctttgatgtaattggcttcgctcggacgagatgctggttgcgaactcgaatctctgttggctgctgagcttcatctgcaccctggtgaccccccctgggctggcatgtggacctggccgaggcattggcaagaggagacaccccaaaaaactcacccctctcgcctataagcagttcatccccaacgtggcggagaagaccctgggggccagcggcagatacgaaggaaagattacaaggaactcggattgctttaaagaattaacccccaattataacccagatattatgtttaaagacgaggagagcaccggggcggaccggctcatgactcagagatgtaaagacaaactgaacgcactcgcgatctccgtgatgaaccagtggccgggggtgaagctgcgggtgacggaggggtgggatgaggacgggcaccacttggaggagtcgctacattatgaggggagggcagtggacatcactacgtcggaccgggaccgcagtaaatacggaatgttgggccgactggcggtggaggccgggttcgactgggtctattacgagtccaaagctcatattcactgttcggtcaaagcagagaactcagtggcggccaagtctggcgggtgcttccctgctggtgccagggtgatggtggaatttggtggcaccaaagcggtgaaagacctgcgaccaggggaccgcgttctctcctccgacccccaagggaatctgctctacagcgacttcctcatgttcatcgaccaggagcgtgacgtcaagaagctcttttacgtcatcgaaacgtctcagagaaaaattcggttgaccgcggcccatctactttttgtggcccagaccaaggtcaacggcaccaggtcgttcaagtctgtctttgccagcaacatccaaccaggagatctcatttatacagcagaatcccaagaccatgaccttgaagggcggggaaagtggagaaggttgatcttgagggaggacactggagcttatgcgcctctaactgcccatgggactgtggttatagaccaggtattggcctcctgctatgcagtcattgaggaacacacctgggcacacctcgcatttgcgccactgaggtttggcatgagcctctcctcttatatttaccccagagactccagtcctccatcaggccttcagcctcaccaccaagttgaccttcagtctcaccatcaagttgatcttcagtctcaccaccaagttgaccttcagtctcaccaccaacttgaaggcatccactggtactcccagctactgtatcagatagggacttggcttttggacagtagctccctgcacccactgggcatggcaacgaaatccagttga

SEQ ID NO: 8 - protein, 446 aa, Xenopus laevis
MLVANSNLCWLLSFICTLVTPPGLACGPGRGIGKRRHPKKLTPLAYKQFIPNVAEKTLGASGRYEGKITRNSDCFKELTPNYNPDIMFKDEESTGADRLMTQRCKDKLNALAISVMNQWPGVKLRVTEGWDEDGHHLEESLHYEGRAVDITTSDRDRSKYGMLGRLAVEAGFDWVYYESKAHIHCSVKAENSVAAKSGGCFPAGARVMVEFGGTKAVKDLRPGDRVLSSDPQGNLLYSDFLMFIDQERDVKKLFYVIETSQRKIRLTAAHLLFVAQTKVNGTRSFKSVFASNIQPGDLIYTAESQDHDLEGRGKWRRLILREDTGAYAPLTAHGTVVIDQVLASCYAVIEEHTWAHLAFAPLRFGMSLSSYIYPRDSSPPSGLQPHHQVDLQSHHQVDLQSHHQVDLQSHHQLEGIHWYSQLLYQIGTWLLDSSSLHPLGMATKSS

SEQ ID NO: 9 - DNA, 1261 bp, Homo sapiens
cccagcgctgcaaggaccgcctgaactcgctggctatctcggtgatgaaccagtggcccggtgtgaagctgcgggtgaccgagggctgggacgaggacggccaccactcagaggagtccctgcattatgagggccgcgcggtggacatcaccacatcagaccgcgaccgcaataagtatggactgctggcgcgcttggcagtggaggccggctttgactgggtgtattacgagtcaaaggcccacgtgcattgctccgtcaagtccgagcactcggccgcagcaacgacgggcggctgcttccctgccggagcccaggtacgcctggagagtggggcgcgtgtggccttgtcagccgtgaggccgggagaccgtgtgctggccatgggggaggatgggagccccaccttcagcgatgtgctcattttcctggaccgcgagcctcacaggctgagagccttccaggtcatcgagactcaggaccccccacgccgcctggcactcacacccgctcacctgctctttacggctgacaatcacacggagccggcagcccgcttccgggccacatttgccagccacgtgcagcctggccagtacgtgctggtggctggggtgccaggcctgcagcctgcccgcgtggcagctgtctctacacacgtggccctcggggcctacgccccgctcacaaagcatgggacactggtggtggaggatgtggtggcatcctgcttcgcggccgtggctgaccaccacctggctcagttggccttctggcccctgagactctttcacagcttggcatggggcagctggaccccgggggagggtgtgcattggtacccccagctgctctaccgcctggggcgtctcctgctagaagagggcagcttccacccactgggcatgtccggggcagggagctgaaaggactccaccgctgccctcctggaactgctgtactgggtccagaagcctctcagccaggagggagctggccctggaagggacctgagctgggggacactggctcctgccatctcctctgccatgaagatacaccattgagacttgactgggcaacaccagcgtcccccacccccgtcgtggtgtagtcatagagctgcaagctgagctggcgaggggatggttgttgacccctctctcctagagaccttgaggctggcacggcgactcccaactcagcctgctctcactacgagttttcatactctgcctcccccattgggagggcccattcc

SEQ ID NO: 10 - protein, 293 aa, Homo sapiens
MNQWPGVKLRVTEGWDEDGHHSEESLHYEGRAVDITTSDRDRNKYGLLARLAVEAGFDWVYYESKAHVHCSVKSEHSAAATTGGCFPAGAQVRLESGARVALSAVRPGDRVLAMGEDGSPTFSDVLIFLDREPHRLRAFQVIETQDPPRRLALTPAHLLFTADNHTEPAARFRATFASHVQPGQYVLVAGVPGLQPARVAAVSTHVALGAYAPLTKHGTLVVEDVVASCFAAVADHHLAQLAFWPLRLFHSLAWGSWTPGEGVHWYPQLLYRLGRLLLEEGSFHPLGMSGAGS

SEQ ID NO: 11 - DNA, 1277 bp, Homo sapiens
ccggcgcctcatgacccagcgctgcaaggaccgcctgaactcgctggctatctcggtgatgaaccagtggcccggtgtgaagctgcgggtgaccgagggctgggacgaggacggccaccactcagaggagtccctgcattatgagggccgcgcggtggacatcaccacatcagaccgcgaccgcaataagtatggactgctggcgcgcttggcagtggaggccggctttgactgggtgtattacgagtcaaaggcccacgtgcattgctccgtcaagtccgagcactcggccgcagccaagacgggcggctgcttccctgccggagcccaggtacgcctggagagtggggcgcgtgtggccttgtcagccgtgaggccgggagaccgtgtgctggccatgggggaggatgggagccccaccttcagcgatgtgctcattttcctggaccgcgagccccacaggctgagagccttccaggtcatcgagactcaggaccccccacgccgcctggcactcacacccgctcacctgctctttacggctgacaatcacacggagccggcagcccgcttccgggccacatttgccagccacgtgcagcctggccagtacgtgctggtggctggggtgccaggcctgcagcctgcccgcgtggcagctgtctctacacacgtggccctcggggcctacgccccgctcacaaagcatgggacactggtggtggaggatgtggtggcatcctgcttcgcggccgtggctgaccaccacctggctcagttggccttctggcccctgagactctttcacagcttggcatggggcagctggaccccgggggagggtgtgcattggtacccccagctgctctaccgcctggggcgtctcctgctagaagagggcagcttccacccactgggcatgtccggggcagggagctgaaaggactccaccgctgccctcctggaactgctgtactgggtccagaagcctctcagccaggagggagctggccctggaagggacctgagctgggggacactggctcctgccatctcctctgccatgaagatacaccattgagacttgactgggcaacaccagcgtcccccacccgcgtcgtggtgtagtcatagagctgcaagctgagctggcgaggggatggttgttgacccctctctcctagagaccttgaggctggcacggcgactcccaactcagcctgctctcactacgagttttcatactctgcctcccccattgggagggcccattcccc

SEQ ID NO: 12 - protein, 312 aa, Homo sapiens
RRLMTQRCKDRLNSLAISVMNQWPGVKLRVTEGWDEDGHHSEESLHYEGRAVDITTSDRDRNKYGLLARLAVEAGFDWVYYESKAHVHCSVKSEHSAAAKTGGCFPAGAQVRLESGARVALSAVRPGDRVLAMGEDGSPTFSDVLIFLDREPHRLRAFQVIETQDPPRRLALTPAHLLFTADNHTEPAARFRATFASHVQPGQYVLVAGVPGLQPARVAAVSTHVALGAYAPLTKHGTLVVEDVVASCFAAVADHHLAQLAFWPLRLFHSLAWGSWTPGEGVHWYPQLLYRLGRLLLEEGSFHPLGMSGAGS

SEQ ID NO: 13 - DNA, 1191 bp, Mus musculus
atggctctgccggccagtctgttgcccctgtgctgcttggcactcttggcactatctgcccagagctgcgggccgggccgaggaccggttggccggcggcgttatgtgcgcaagcaacttgtgcctctgctatacaagcagtttgtgcccagtatgcccgagcggaccctgggcgcgagtgggccagcggaggggagggtaacaagggggtcggagcgcttccgggacctcgtacccaactacaaccccgacataatcttcaaggatgaggagaacagcggcgcagaccgcctgatgacagagcgttgcaaagagcgggtgaacgctctagccatcgcggtgatgaacatgtggcccggagtacgcctacgtgtgactgaaggctgggacgaggacggccaccacgcacaggattcactccactacgaaggccgtgccttggacatcaccacgtctgaccgtgaccgtaataagtatggtttgttggcgcgcctagctgtggaagccggattcgactgggtctactacgagtcccgcaaccacatccacgtatcggtcaaagctgataactcactggcggtccgagccggaggctgctttccgggaaatgccacggtgcgcttgcggagcggcgaacggaaggggctgagggaactacatcgtggtgactgggtactggccgctgatgcagcgggccgagtggtacccacgccagtgctgctcttcctggaccgggatctgcagcgccgcgcctcgttcgtggctgtggagaccgagcggcctccgcgcaaactgttgctcacaccctggcatctggtgttcgctgctcgcgggccagcgcctgctccaggtgactttgcaccggtgttcgcgcgccgcttacgtgctggcgactcggtgctggctcccggcggggacgcgctccagccggcgcgcgtagcccgcgtggcgcgcgaggaagccgtgggcgtgttcgcaccgctcactgcgcacgggacgctgctggtcaacgacgtcctcgcctcctgctacgcggttctagagagtcaccagtgggcccaccgcgccttcgcccctttgcggctgctgcacgcgctcggggctctgctccctgggggtgcagtccagccgactggcatgcattggtactctcgcctcctttaccgcttggccgaggagttaatgggctga

SEQ ID NO: 14 - protein, 396 aa, Mus musculus [sequence omitted]

SEQ ID NO: 15 - DNA, 3600 bp, Homo sapiens
cccagactccagccctggaccgcgcatcccgagcccagcgcccagacagagtgtccccacaccctcctctgagacgccatgttcaactcgatgaccccaccaccaatcagtagctatggcgagccctgctgtctccggcccctccccagtcagggggcccccagtgtggggacagaaggactgtctggcccgcccttctgccaccaagctaacctcatgtccggcccccacagttatgggccagccagagagaccaacagctgcaccgagggcccactcttttcttctccccggagtgcagtcaagttgaccaagaagcgggcactgtccatctcacctctgtcggatgccagcctggacctgcagacggttatccgcacctcacccagctccctcgtagctttcatcaactcgcgatgcacatctccaggaggctcctacggtcatctctccattggcaccatgagcccatctctgggattcccagcccagatgaatcaccaaaaagggccctcgccttcctttggggtccagccttgtggtccccatgactctgcccggggtgggatgatcccacatcctcagtcccggggacccttcccaacttgccagctgaagtctgagctggacatgctggttggcaagtgccgggaggaacccttggaaggtgatatgtccagccccaactccacaggcatacaggatcccctgttggggatgctggatgggcgggaggacctcgagagagaggagaagcgtgagcctgaatctgtgtatgaaactgactgccgttgggatggctgcagccaggaatttgactcccaagagcagctggtgcaccacatcaacagcgagcacatccacggggagcggaaggagttcgtgtgccactgggggggctgctccagggagctgaggcccttcaaagcccagtacatgctggtggttcacatgcgcagacacactggcgagaagccacacaagtgcacgtttgaagggtgccggaagtcatactcacgcctcgaaaacctgaagacgcacctgcggtcacacacgggtgagaagccatacatgtgtgagcacgagggctgcagtaaagccttcagcaatgccagtgaccgagccaagcaccagaatcggacccattccaatgagaagccgtatgtatgtaagctccctggctgcaccaaacgctatacagatcctagctcgctgcgaaaacatgtcaagacagtgcatggtcctgacgcccatgtgaccaaacggcaccgtggggatggccccctgcctcgggcaccatccatttctacagtggagcccaagagggagcgggaaggaggtcccatcagggaggaaagcagactgactgtgccagagggtgccatgaagccacagccaagccctggggcccagtcatcctgcagcagtgaccactccccggcagggagtgcagccaatacagacagtggtgtggaaatgactggcaatgcagggggcagcactgaagacctctccagcttggacgagggaccttgcattgctggcactggtctgtccactcttcgccgccttgagaacctcaggctggaccagctacatcaactccggccaatagggacccggggtctcaaactgcccagcttgtcccacaccggtaccactgtgtcccgccgcgtgggccccccagtctctcttgaacgccgcagcagcagctccagcagcatcagctctgcctatactgtcagccgccgctcctccctggcctctcctttcccccctggctccccaccagagaatggagcatcctccctgcctggccttatgcctgcccagcactacctgcttcgggcaagatatgcttcagccagagggggtggtacttcgcccactgcagcatccagcctggatcggataggtggtcttcccatgcctccttggagaagccgagccgagtatccaggatacaaccccaatgcaggggtcacccggagggccagtgacccagcccaggctgctgaccgtcctgctccagctagagtccagaggttcaagagcctgggctgtgtccataccccacccactgtggcagggggaggacagaactttgatccttacctcccaacctctgtctactcaccacagccccccagcatcactgagaatgctgccatggatgctagagggctacaggaagagccagaagttgggacctccatggtgggcagtggtctgaacccctatatggacttcccacctactgatactctgggatatgggggacctgaaggggcagcagctgagccttatggagcgaggggtccaggctctctgcctcttgggcctggtccacccaccaactatggccccaacccctgtccccagcaggcctcatatcctgaccccacccaagaaacatggggtgagttcccttcccactctgggctgtacccaggccccaaggctctaggtggaacctacagccagtgtcctcgacttgaacattatggacaagtgcaagtcaagccagaacaggggtgcccagtggggtctgactccacaggactggcaccctgcctcaatgcccaccccagtgaggggcccccacatccacagcctctcttttcccattacccccagccctctcctccccaatatctccagtcaggcccctatacccagccaccccctgattatcttccttcagaacccaggccttgcctggactttgattcccccacccattccacagggcagctcaaggctcagcttgtgtgtaattatgttcaatctcaacaggagctactgtgggagggtgggggcagggaagatgcccccgcccaggaaccttcctaccagagtcccaagtttctggggggttcccaggttagcccaagccgtgctaaagctccagtgaacacatatggacctggctttggacccaacttgcccaatcacaagtcaggttcctatcccaccccttcaccatgccatgaaaattttgtagtgggggcaaatagggcttcacatagggcagcagcaccacctcgacttctgcccccattgcccacttgctatgggcctctcaaagtgggaggcacaaaccccagctgtggtcatcctgaggtgggcaggctaggagggggtcctgccttgtaccctcctcccgaaggacaggtatgtaaccccctggactctcttgatcttgacaacactcagctggactttgtggctattctggatgagccccaggggctgagtcctcctccttcccatgatcagcggggcagctctggacataccccacctccctctgggccccccaacatggctgtgggcaacatgagtgtcttactgagatccctacctggggaaacagaattcctcaactctagtgcctaaagagtagggaatctcatccatcacagatcgcatttcctaaggggtttctatccttccagaaaaattgggggagctgcagtcccctgcacaagatgccccagggatgggaggtatgggctgggggctatgtatagtctgtatacgttttgaggagaaatttgataatgacactgtttcctgataataaaggaactgcatcag

SEQ ID NO: 16 - protein, 1106 aa, Homo sapiens
MFNSMTPPPISSYGEPCCLRPLPSQGAPSVGTEGLSGPPFCHQANLMSGPHSYGPARETNSCTEGPLFSSPRSAVKLTKKRALSISPLSDASLDLQTVIRTSPSSLVAFINSRCTSPGGSYGHLSIGTMSPSLGFPAQMNHQKGPSPSFGVQPCGPHDSARGGMIPHPQSRGPFPTCQLKSELDMLVGKCREEPLEGDMSSPNSTGIQDPLLGMLDGREDLEREEKREPESVYETDCRWDGCSQEFDSQEQLVHHINSEHIHGERKEFVCHWGGCSRELRPFKAQYMLVVHMRRHTGEKPHKCTFEGCRKSYSRLENLKTHLRSHTGEKPYMCEHEGCSKAFSNASDRAKHQNRTHSNEKPYVCKLPGCTKRYTDPSSLRKHVKTVHGPDAHVTKRHRGDGPLPRAPSISTVEPKREREGGPIREESRLTVPEGAMKPQPSPGAQSSCSSDHSPAGSAANTDSGVEMTGNAGGSTEDLSSLDEGPCIAGTGLSTLRRLENLRLDQLHQLRPIGTRGLKLPSLSHTGTTVSRRVGPPVSLERRSSSSSSISSAYTVSRRSSLASPFPPGSPPENGASSLPGLMPAQHYLLRARYASARGGGTSPTAASSLDRIGGLPMPPWRSRAEYPGYNPNAGVTRRASDPAQAADRPAPARVQRFKSLGCVHTPPTVAGGGQNFDPYLPTSVYSPQPPSITENAAMDARGLQEEPEVGTSMVGSGLNPYMDFPPTDTLGYGGPEGAAAEPYGARGPGSLPLGPGPPTNYGPNPCPQQASYPDPTQETWGEFPSHSGLYPGPKALGGTYSQCPRLEHYGQVQVKPEQGCPVGSDSTGLAPCLNAHPSEGPPHPQPLFSHYPQPSPPQYLQSGPYTQPPPDYLPSEPRPCLDFDSPTHSTGQLKAQLVCNYVQSQQELLWEGGGREDAPAQEPSYQSPKFLGGSQVSPSRAKAPVNTYGPGFGPNLPNHKSGSYPTPSPCHENFVVGANRASHRAAAPPRLLPPLPTCYGPLKVGGTNPSCGHPEVGRLGGGPALYPPPEGQVCNPLDSLDLDNTQLDFVAILDEPQGLSPPPSHDQRGSSGHTPPPSGPPNMAVGNMSVLLRSLPGETEFLNSSA

SEQ ID NO: 17 - DNA, 4909 bp, Homo sapiens
gcgcccgccgccttacctgaggccgccatccacgccggttgagtcgcgttctgccgcctcccgcctgtggtgcctcctgaactgcgtccgccgtctagtgaagttcgtggactcctacaataatgctataaatgcatagaagaaaagacacaggactgtgaaagaaagtgatgatgcgatgtctaaaacgttcaaggcaccgcatctgtgatcaagaatacatgtgctgctttaccgacacatcaaagagcaaggattgccacccaggacgatgagcggctgagatggagacgtctgcctcagccactgcctccgagaagcaagaagccaaaagtgggatcctggaggccgctggcttccccgacccgggtaaaaaggcctctcctttggtggtggctgcagcggcagcagcagcggtagctgcccaaggagcccagccttcaccttcccccaccccatcaaccccgtggcctaccagcagattctgagccagcagaggggtctggggtcagcctttggacacacaccacccctgatccagccctcacccaccttcctggcccagcagcccatggccctcacctccatcaatgccacgcccacccagctcagcagcagcagcaactgtctgagtgacaccaaccagaacaagcagagcagtgagtcggccgtcagcagcaccgtcaaccctgtcgccattcacaagcgcagcaaggtcaagaccgagcctgagggcctgcggccggcctcccctctggcgctgacgcaggagcagctggctgacctcaaggaagatctggacagggatgactgtaagcaggaggctgaggtggtcatctatgagaccaactgccactgggaagactgcaccaaggagtacgacacccaggagcagctggtgcatcacatcaacaacgagcacatccacggggagaagaaggagtttgtgtgccgctggcaggcctgcacgcgggagcagaagcccttcaaggcgcagtacatgctggtggtgcacatgcggcgacacacgggcgagaagccccacaagtgcacgttcgagggctgctcgaaggcctactcccgcctggagaacctgaagacacacctgcggtcccacaccggggagaagccatatgtgtgtgagcacgagggctgcaacaaagccttctccaacgcctcggaccgcgccaagcaccagaatcgcacccactccaacgagaaaccctacatctgcaagatcccaggctgcaccaagagatacacagaccccagctctctccggaagcatgtgaaaacggtccacggcccagatgcccacgtcaccaagaagcagcgcaatgacgtgcacctccgcacaccgctgctcaaagagaatggggacagtgaggccggcacggagcctggcggcccagagagcaccgaggccagcagcaccagccaggccgtggaggactgcctgcacgtcagagccatcaagaccgagagctccgggctgtgtcagtccagccccggggcccagtcgtcctgcagcagcgagccctctcctctgggcagtgcccccaacaatgacagtggcgtggagatgccggggacggggcccgggagcctgggagacctgacggcactggatgacacacccccaggggccgacacctcagccctggctgccccctccgctggtggcctccagctgcgcaaacacatgaccaccatgcaccggttcgagcagctcaagaaggagaagctcaagtcactcaaggattcctgctcatgggccgggccgactccacacacgcggaacaccaagctgcctcccctcccgggaagtggctccatcctggaaaacttcagtggcagtgggggcggcgggcccgcggggctgctgccgaacccgcggctgtcggagctgtccgcgagcgaggtgaccatgctgagccagctgcaggagcgccgcgacagctccaccagcacggtcagctcggcctacaccgtgagccgccgctcctccggcatctccccctacttctccagccgccgctccagcgaggcctcgcccctgggcgccggccgcccgcacaacgcgagctccgctgactcctacgaccccatctccacggacgcgtcgcggcgctcgagcgaggccagccagtgcagcggcggctccgggctgctcaacctcacgccggcgcagcagtacagcctgcgggccaagtacgcggcagccactggcggccccccgcccactccgctgccgggcctggagcgcatgagcctgcggaccaggctggcgctgctggacgcggccgagggcacgctgcccgccggctgcccacgcccactggggccgcggcgtggcagcgacgggccgacctatggccacggccacgcgggggctgcgcccgccttcccccacgaggctccaggcggcggaaccaggcgggccagcgaccctgtgcggcggcccgatgccctgtccctgccgcgggtgcagcgcttccacagcacccacaacgtgaaccccggcccgctgccgccctgtgccgacaggcgaggcctccgcctgcagagccacccgagcaccgacggcggcctggcccgcggcgcctactcgccccggccgcctagcatcagcgagaacgtggcgatggaggccgtggcggcaggagtggacggcgcggggcccgaggccgacctggggctgccggaggacgacctggtgcttccagacgacgtggtgcagtacatcaaggcgcacgccagtggcgctctggacgagggcaccgggcaggtgtatcccacggaaagcactggcttctctgacaaccccagactacccagcccggggctgcacggccagcgcaggatggtggctgcggactccaacgtgggcccctccgcccctatgctgggaggatgccagttaggctttggggcgccctccagcctgaacaaaaataacatgcctgtgcagtggaatgaggtgagctccggcaccgtagactccctggccagccaggtgaagcctccaccctttcctcagggcaacctggcggtggtgcagcagaagcctgcctttggccagtacccgggctacagtccgcaaggcctacaggctagccctgggggcctggacagcacgcagccacacctgcagccccgcagcggagccccctcccagggcatccccagggtaaactacatgcagcagctgcgacagccagtggcaggcagccagtgtcctggcatgactaccactatgagcccccatgcctgctatggccaagtccacccccagctgagccccagcaccatcagtggggccctcaaccagttcccccaatcctgcagcaacatgccagccaagccagggcatctggggcaccctcagcagacagaagtggcacctgaccccaccacgatgggcaatcgccacagggaacttggggtccccaattcagccctggctggagtgccgccacctcacccagtccagagctacccacagcagagccatcacctggcagcctccatgagccaggagggctaccaccaggtccccagccttctgcctgcccgccagcctggcttcatggagccccaaacaggcccgatgggggtggctacagcaggctttggcctagtgcagccccggcctcccctcgagcccagccccactggccgccaccgtggggtacgtgctgtgcagcagcagctggcctacgccagggccacaggccatgccatggctgccatgccgtccagtcaggaaacagcagaggctgtgcccaagggagcgatgggcaacatggggtcggtgcctccccagccgcctccgcaggacgcaggtggggccccggaccacagcatgctctactactacggccagatccacatgtacgaacaggatggaggcctggagaacctcgggagctgccaggtcatgcggtcccagccaccacagccacaggcctgtcaggacagcatccagccccagcccttgccctcaccaggggtcaaccaggtgtccagcactgtggactcccagctcctggaggccccccagattgacttcgatgccatcatggatgatggcgatcactcgagtttgttctcgggtgctctgagccccagcctcctccacagcctctcccagaactcctcccgcctcaccaccccccgaaactccttgaccctgccctccatccccgcaggcatcagcaacatggctgtcggggacatgagctccatgctcaccagcctcgccgaggagagcaagttcctgaacatgatgacctagaggcccgagcgcctggtgctgagtgcacccggaggggtcatcgctgcccagagcctggggattccagctgtcttgtctttttccaaaaaagtgttaaataggcttgaggggttgttgcgcaatggccgcttcagatgacagatgttgtaagagaaggtttatgggcatcctctctggtcttttggattattcctcagaacaatgaaaaaagtctccataggacaggaaggaatgcaaaactcatttacacagtgctttccagcctttggtgcttacaggaccgcgctgttccggcttcttcacggctgacattcggctaacgagggattactttggccaaaacctttcaaaggatatgcagaaagatggtagggagcatttgggtttgaatctgaatgctatactggatactctgctccggaaagatgagctttttattctactacttggaaggaaaaggaattcctctatgaagcctaactcttgaggtctctaacataccttgtcatagaggaaaagcacagattatacctggatgattcaggagagtgtatatgaatgaataaggcatccaagtatatatgaatgaataaagtatgtaagtatcaccag

SEQ ID NO: 18 - protein, 1241 aa, Homo sapiens
MALTSINATPTQLSSSSNCLSDTNQNKQSSESAVSSTVNPVAIHKRSKVKTEPEGLRPASPLALTQEQLADLKEDLDRDDCKQEAEVVIYETNCHWEDCTKEYDTQEQLVHHINNEHIHGEKKEFVCRWQACTREQKPFKAQYMLVVHMRRHTGEKPHKCTFEGCSKAYSRLENLKTHLRSHTGEKPYVCEHEGCNKAFSNASDRAKHQNRTHSNEKPYICKIPGCTKRYTDPSSLRKHVKTVHGPDAHVTKKQRNDVHLRTPLLKENGDSEAGTEPGGPESTEASSTSQAVEDCLHVRAIKTESSGLCQSSPGAQSSCSSEPSPLGSAPNNDSGVEMPGTGPGSLGDLTALDDTPPGADTSALAAPSAGGLQLRKHMTTMHRFEQLKKEKLKSLKDSCSWAGPTPHTRNTKLPPLPGSGSILENFSGSGGGGPAGLLPNPRLSELSASEVTMLSQLQERRDSSTSTVSSAYTVSRRSSGISPYFSSRRSSEASPLGAGRPHNASSADSYDPISTDASRRSSEASQCSGGSGLLNLTPAQQYSLRAKYAAATGGPPPTPLPGLERMSLRTRLALLDAAEGTLPAGCPRPLGPRRGSDGPTYGHGHAGAAPAFPHEAPGGGTRRASDPVRRPDALSLPRVQRFHSTHNVNPGPLPPCADRRGLRLQSHPSTDGGLARGAYSPRPPSISENVAMEAVAAGVDGAGPEADLGLPEDDLVLPDDVVQYIKAHASGALDEGTGQVYPTESTGFSDNPRLPSPGLHGQRRMVAADSNVGPSAPMLGGCQLGFGAPSSLNKNNMPVQWNEVSSGTVDSLASQVKPPPFPQGNLAVVQQKPAFGQYPGYSPQGLQASPGGLDSTQPHLQPRSGAPSQGIPRVNYMQQLRQPVAGSQCPGMTTTMSPHACYGQVHPQLSPSTISGALNQFPQSCSNMPAKPGHLGHPQQTEVAPDPTTMGNRHRELGVPNSALAGVPPPHPVQSYPQQSHHLAASMSQEGYHQVPSLLPARQPGFMEPQTGPMGVATAGFGLVQPRPPLEPSPTGRHRGVRAVQQQLAYARATGHAMAAMPSSQETAEAVPKGAMGNMGSVPPQPPPQDAGGAPDHSMLYYYGQIHMYEQDGGLENLGSCQVMRSQPPQPQACQDSIQPQPLPSPGVNQVSSTVDSQLLEAPQIDFDAIMDDGDHSSLFSGALSPSLLHSLSQNSSRLTTPRNSLTLPSIPAGISNMAVGDMSSMLTSLAEESKFLNMMT

SEQ ID NO: 19 - DNA, 5054 bp, Homo sapiens
cgatactacgtgggcatttttggtcgaagagagctgaagtaatgagaagacatcatggaggcccagtcccacagctccacgaccactgaaaagaaaaaagttgagaattccatagtgaagtgctccactcgaacagatgtgagcgagaaagccgttgcctccagcaccacttctaatgaggatgaaagtcctggacagacttatcacagagagagaagaaacgcaatcactatgcagccacagaatgtccaggggctcagcaaagtcagtgaggaaccttcaacatcgagtgacgagagggcctcattgatcaagaaagagatccatgggtccctgccacacgtggcggagccctctgtgccgtaccgcgggacggtgtttgccatggaccccaggaatggttacatggagccccactaccaccctcctcatcttttccctgccttccatcctcctgtaccaattgatgccagacatcatgagggccgttaccattacgatccatctccgattcctccattgcatatgacttccgccttatctagtagccctacgtatccggacctgcccttcattaggatctccccacaccggaaccccgctgctgcttccgagtctcccttcagccctccacatccctacattaatccctacatggactatatccgctccttgcacagcagcccatcgctctccatgatctcagcaacccgtgggctgagccctacagatgcgccccatgcaggagtcagcccagcagaatactatcatcagatggccctgctaactggccagcgcagcccctatgcagacattattccctcagctgccaccgccggcacgggggccatccacatggaatatcttcatgctatggatagcaccagattctccagccccaggctgtcagccaggccgagccgaaaacgtacactgtccatatcaccactctccgatcatagctttgaccttcagaccatgataaggacgtctcccaactccttggtcacgattctcaataattcccgtagcagctcttcagcaagtggctcctatggtcacttatctgcaagtgcaatcagccctgccttgagcttcacctactcttccgcgcccgtctctctccacatgcatcagcagatcctaagccgacaacagagcttaggttcagcctttggacacagccctccactcatccaccctgccccaacttttccaacacagaggcctattccagggatccctacggttctgaaccccgtccaggtcagctccggcccttctgagtcctcacagaacaagcccacgagtgagtctgcagtgagcagcactggtgacccgatgcacaacaagaggtccaagatcaaacccgatgaagacctccccagcccaggggctcgggggcagcaggaacagcccgaaggaacaacccttgtcaaggaggaaggggacaaagatgaaagcaaacaggagcctgaagtcatctatgagacaaactgccactgggaaggctgcgcgagggagttcgacacccaagagcagcttgtgcaccatataaataacgaccatattcatggagagaagaaggagttcgtgtgcaggtggctggactgctcaagagagcagaaacccttcaaagcccagtatatgttggtagtgcatatgagaagacacacgggcgagaagcctcacaaatgcacttttgaaggttgcacaaaggcctactcgagactagaaaacttgaaaacacacttgagatctcacactggagagaaaccatacgtctgtgagcacgaaggttgcaacaaggctttctcaaatgcctctgatcgcgccaaacaccaaaacagaacgcattccaatgagaaaccatatgtgtgcaaaatcccaggctgcactaagcgttacacagacccaagctccctccggaaacatgtgaagacagtgcatggcccagaggctcatgtcaccaagaagcagcgaggggacatccatcctcggccgccacccccgagagattccggcagccattcacagtccaggtcgcctggccgaccgactcagggagcccttggtgagcagcaggacctcagcaacactacctcaaagcgggaagaatgcctccaggtgaaaaccgtcaaggcagagaagccaatgacatctcagccaagccctggtggtcagtcttcatgcagcagccaacagtcccccatcagcaactattccaacagtgggctcgagcttcctctgaccgatggaggtagtataggagacctcagtgccatcgatgaaaccccaatcatggactcaaccatttccactgcaaccacagcccttgctttgcaagccaggagaaacccggcagggaccaaatggatggagcacgtaaaactagaaaggctaaaacaagtgaatggaatgtttccgcgactgaaccccattctaccccctaaagcccctgcggtctctcctctcataggaaatggcacacagtccaacaacacctgcagcttgggtgggcccatgacgcttctcccgggcagaagcgacctctctggggtggacgtcactatgctgaacatgctcaacagaagggacagcagcgccagcaccatcagctcggcctacctgagcagccgccgctcctcagggatctcgccctgcttctccagccgccgctccagcgaggcgtcacaggccgagggccggccgcagaacgtgagcgtggccgactcctacgaccccatctccaccgacgcctcgcgccgctccagcgaagccagccagagcgacggcctgcccagcctgctcagcctcacgcccgcccagcagtaccgcctcaaggccaagtacgcggctgccacaggagggccgccgccgacgcccctgcccaacatggagaggatgagcctgaagacgcgcctggcgctgctcggggatgccctcgagcctggcgtggccctgcctccagttcatgccccgaggaggtgcagcgacgggggagcccacggctacgggcggcgccacctgcagccgcacgatgcgctgggccacggcgtgaggagggccagcgacccggtgcggacaggctccgagggcctggccctgcctcgtgtgccgcgcttcagcagcctcagcagctgcaaccccccggcgatggccacgtccgcggagaagcgcagtctcgtgcttcagaattacacgcggcccgagggcggccagtcccgaaacttccactcgtccccctgtcctcccagcatcaccgagaacgtcaccctggagtccctgaccatggacgctgatgccaacctgaacgatgaggatttcctgccggacgacgtggtgcagtatttaaattcccagaaccaagcagggtacgagcagcacttccccagcgccctcccggacgacagcaaagtgccccacgggcccggtgactttgacgcgcccgggctgccagacagccacgctggccagcagttccatgccctcgagcagccctgccccgagggcagcaaaaccgacctgcccattcagtggaacgaagtcagctccggaagcgccgacctgtcctcctccaagctcaagtgtgggccgcggcccgctgtgccgcagactcgcgcctttgggttctgcaacggcatggtcgtccacccgcagaaccccttgaggagcgggcctgctgggggctatcagaccctcggggagaacagcaacccctacggtggcccagagcacttgatgctccacaacagccccggaagtggcaccagtggaaacgccttccatgaacagccctgtaaggccccgcagtatgggaactgtctcaacaggcagccagtggcccctggtgcactcgacggtgcctgtggtgccgggattcaagcctcaaagctgaagagcacccccatgcaagggagcgggggccagctgaatttcggcctgccggtagcgccaaatgagtcagctggcagcatggtgaatggcatgcagaaccaggacccagtgggacaggggtacctggctcaccagctcctcggcgacagcatgcagcacccgggggcaggccgccccggtcagcagatgcttgggcagattagtgctacctcacacatcaacatctaccaagggccagagagctgcctgccaggggctcacggcatgggcagccagccgtcaagcttggcagttgtcaggggctaccagccatgtgccagctttgggggcagcaggcgccaggctatgccgagggacagccttgctctgcagtcaggacagctcagtgacacaagtcagacctgcagggtgaatggtatcaagatggagatgaaagggcagccccatccgctgtgctctaatctgcagaattactctggtcagttctatgaccaaaccgtgggcttcagtcagcaagacacgaaagctggttcattctctatttcagacgccagctgcctgctacaggggaccagcgccaaaaactctgagttactttccccaggtgctaatcaggtgacaagcacagtggacagcctcgacagccatgacctggaaggggtacagattgacttcgatgccatcatagacgatggggaccactccagcctgatgtcgggggccctgagcccaagtatcattcagaacctttcccatagctcctcccgcctcaccacgcctcgggcgtccctcccattcccagcgctgtccatgagcaccaccaacatggctatcggggacatgagttctttgctgacctccctagcggaagaaagcaaattccttgcagttatgcaataggctttaggaaaaaaagactgcaaccaacggaaatcaataggagttgaagagattaaactgactttgttttggctgtttttttagttctgtatgtattttagcaatctcatctcacctaactgagatgtgtttcaattatattccttttatggaaaaggactctgaaaaaccctaaagtattctagggagaaactgtcttccatttcagttttgaatcagtattgttacactcaaaccaccctctttttaaaaaaaaa

SEQ ID NO: 20 - protein, 1597 aa, Homo sapiens
  VARIANT 1597: Xaa = Any Amino Acid
MEAQSHSSTTTEKKKVENSIVKCSTRTDVSEKAVASSTTSNEDESPGQTYHRERRNAITMQPQNVQGLSKVSEEPSTSSDERASLIKKEIHGSLPHVAEPSVPYRGTVFAMDPRNGYMEPHYHPPHLFPAFHPPVPIDARHHEGRYHYDPSPIPPLHMTSALSSSPTYPDLPFIRISPHRNPAAASESPFSPPHPYINPYMDYIRSLHSSPSLSMISATRGLSPTDAPHAGVSPAEYYHQMALLTGQRSPYADIIPSAATAGTGAIHMEYLHAMDSTRFSSPRLSARPSRKRTLSISPLSDHSFDLQTMIRTSPNSLVTILNNSRSSSSASGSYGHLSASAISPALSFTYSSAPVSLHMHQQILSRQQSLGSAFGHSPPLIHPAPTFPTQRPIPGIPTVLNPVQVSSGPSESSQNKPTSESAVSSTGDPMHNKRSKIKPDEDLPSPGARGQQEQPEGTTLVKEEGDKDESKQEPEVIYETNCHWEGCAREFDTQEQLVHHINNDHIHGEKKEFVCRWLDCSREQKPFKAQYMLVVHMRRHTGEKPHKCTFEGCTKAYSRLENLKTHLRSHTGEKPYVCEHEGCNKAFSNASDRAKHQNRTHSNEKPYVCKIPGCTKRYTDPSSLRKHVKTVHGPEAHVTKKQRGDIHPRPPPPRDSGSHSQSRSPGRPTQGALGEQQDLSNTTSKREECLQVKTVKAEKPMTSQPSPGGQSSCSSQQSPISNYSNSGLELPLTDGGSIGDLSAIDETPIMDSTISTATTALALQARRNPAGTKWMEHVKLERLKQVNGMFPRLNPILPPKAPAVSPLIGNGTQSNNTCSLGGPMTLLPGRSDLSGVDVTMLNMLNRRDSSASTISSAYLSSRRSSGISPCFSSRRSSEASQAEGRPQNVSVADSYDPISTDASRRSSEASQSDGLPSLLSLTPAQQYRLKAKYAAATGGPPPTPLPNMERMSLKTRLALLGDALEPGVALPPVHAPRRCSDGGAHGYGRRHLQPHDALGHGVRRASDPVRTGSEGLALPRVPRFSSLSSCNPPAMATSAEKRSLVLQNYTRPEGGQSRNFHSSPCPPSITENVTLESLTMDADANLNDEDFLPDDVVQYLNSQNQAGYEQHFPSALPDDSKVPHGPGDFDAPGLPDSHAGQQFHALEQPCPEGSKTDLPIQWNEVSSGSADLSSSKLKCGPRPAVPQTRAFGFCNGMVVHPQNPLRSGPAGGYQTLGENSNPYGGPEHLMLHNSPGSGTSGNAFHEQPCKAPQYGNCLNRQPVAPGALDGACGAGIQASKLKSTPMQGSGGQLNFGLPVAPNESAGSMVNGMQNQDPVGQGYLAHQLLGDSMQHPGAGRPGQQMLGQISATSHINIYQGPESCLPGAHGMGSQPSSLAVVRGYQPCASFGGSRRQAMPRDSLALQSGQLSDTSQTCRVNGIKMEMKGQPHPLCSNLQNYSGQFYDQTVGFSQQDTKAGSFSISDASCLLQGTSAKNSELLSPGANQVTSTVDSLDSHDLEGVQIDFDAIIDDGDHSSLMSGALSPSIIQNLSHSSSRLTTPRASLPFPVAVHEHHQHGYRGHEFFADLPSGRKQIPCSYAIGFRKKRLQPTEINRSX

SEQ ID NO: 21 - DNA, 21 bp, Artificial Sequence
  synthetic
aactccacaggcatacaggat

SEQ ID NO: 22 - DNA, 21 bp, Artificial Sequence
  synthetic
aagatctggacagggatgact

SEQ ID NO: 23 - DNA, 21 bp, Artificial Sequence
  synthetic
aatgatctctgccgccagggg

SEQ ID NO: 24 - DNA, 21 bp, Artificial Sequence
  synthetic
aatgaggatgaaagtcctgga

SEQ ID NO: 25 - DNA, 23 bp, Artificial Sequence
  antisense oligonucleotide
gggatgatcccacatcctcagtc

SEQ ID NO: 26 - DNA, 19 bp, Artificial Sequence
  antisense oligonucleotide
ctggagcagcccccccagt

SEQ ID NO: 27 - DNA, 20 bp, Artificial Sequence
  primer
cttgtagcctctcgtggcag

SEQ ID NO: 28 - DNA, 21 bp, Artificial Sequence
  primer
gaccttcatagcatccgtgag

SEQ ID NO: 29 - RNA, 21 nt, Artificial Sequence
  synthetic
aacuccacaggcauacaggau

SEQ ID NO: 30 - RNA, 21 nt, Artificial Sequence
  synthetic
aacguacgcggaauacaacga

SEQ ID NO: 31 - RNA, 21 nt, Artificial Sequence
  synthetic
aagaucuggacagggaugacu

SEQ ID NO: 32 - RNA, 21 nt, Artificial Sequence
  synthetic
aaugaggaugaaaguccugga

SEQ ID NO: 33 - DNA, 22 bp, Artificial Sequence
  synthetic
cgggcggacactggcgggacgc

SEQ ID NO: 34 - DNA, 25 bp, Artificial Sequence
  synthetic
gcacagaacgcaggtaatgctccat

SEQ ID NO: 35 - DNA, 25 bp, Artificial Sequence
  synthetic
gagattcgagttcgcaaccagcatc